(19) United States
(12) Patent Application Publication
(10) Pub. No.: US 2024/0165112 A1
(43) Pub. Date: May 23, 2024

(54) THERAPY FOR THE TREATMENT OF CANCER

(71) Applicant: Bristol-Myers Squibb Company, Princeton, NJ (US)

(72) Inventors: Patrick Joseph Shaw, Princeton Junction, NJ (US); Veronica Mariotti, Montclair, NJ (US); Philip Vitorino, Redwood City, CA (US); Michael Pickup, San Francisco, CA (US)

(21) Appl. No.: 18/501,563

(22) Filed: Nov. 3, 2023

Related U.S. Application Data
(60) Provisional application No. 63/422,623, filed on Nov. 4, 2022.

(51) Int. Cl. A61K 31/505; A61K 31/7068; A61P 35/00

(52) U.S. Cl. CPC A61K 31/505; A61K 31/7068; A61P 35/00

(57) ABSTRACT

Provided herein are methods of treating and/or managing cancers, which comprise administering to a patient Compound A. Provided herein are also methods of treating and/or managing cancers, which comprise administering to a patient Compound A in combination with nivolumab. Additionally, provided herein are methods of treating and/or managing cancers, which comprise administering to a patient Compound A in combination with chemotherapy.

### CROSS REFERENCE TO RELATED APPLICATIONS

This application claims the benefit of U.S. Provisional Application No. 63/422,623, filed Nov. 4, 2022, the disclosure of which is incorporated herein by reference in its entirety.

### FIELD

Provided herein are therapies for treating and/or managing cancers, which comprise administering to a patient 2 (tert-butylamino)-4-((1R,3R,4R)-3-hydroxy-4-methylcyclohexylamino)-pyrimidine-5-carboxamide (“Compound 1”), or a pharmaceutically acceptable salt, stereoisomer, tautomer, solid form, polymorph, hydrate, clathrate, or solvate thereof (collectively referred to herein as “Compound A”). Further provided herein are combination therapies for treating and/or managing cancers, which comprise administering to a patient 2 (tert-butylamino)-4-((1R,3R,4R)-3-hydroxy-4-methylcyclohexylamino)-pyrimidine-5-carboxamide (“Compound 1”), or a pharmaceutically acceptable salt, stereoisomer, tautomer, solid form, polymorph, hydrate, clathrate, or solvate thereof (collectively referred to herein as “Compound A”), in combination with chemotherapy. Also provided are combination therapies for treating and/or managing cancers, which comprise administering to a patient 2 (tert-butylamino)-4-((1R,3R,4R)-3-hydroxy-4-methylcyclohexylamino)-pyrimidine-5-carboxamide (“Compound 1”), or a pharmaceutically acceptable salt, stereoisomer, tautomer, solid form, polymorph, hydrate, clathrate, or solvate thereof (collectively referred to herein as “Compound A”), in combination with nivolumab. Further provided herein is 2 (tert-butylamino)-4-((1R,3R,4R)-3-hydroxy-4-methylcyclohexylamino)-pyrimidine-5-carboxamide (“Compound 1”), or a pharmaceutically acceptable salt, stereoisomer, tautomer, solid form, polymorph, hydrate, clathrate, or solvate thereof (collectively referred to herein as “Compound A”), for use in said combination therapies. Further, provided herein are pharmaceutical compositions and dosage forms of Compound A. Further provided herein are methods for using the pharmaceutical compositions, and dosage forms of Compound A for treating and/or managing cancers.

### BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

There is an enormous variety of cancers which are described in detail in the medical literature. Examples include cancer of the lung, colon, rectum, prostate, breast, brain, and intestine. The incidence of cancer continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer.

Current cancer therapy may involve surgery, chemotherapy, hormonal therapy and/or radiation treatment to eradicate neoplastic cells in a patient (see, for example, Stockdale, 1998, Medicine, vol. 3, Rubenstein and Federman, eds., Chapter 12, Section IV). Recently, cancer therapy could also involve biological therapy or immunotherapy. All of these approaches may pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells. Certain biological and other therapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A number of cancer chemotherapeutics act by inhibiting DNA synthesis, either directly or indirectly by inhibiting the biosynthesis of deoxyribonucleotide triphosphate precursors, to prevent DNA replication and concomitant cell division. Gilman et al., Goodman and Gilman's: The Pharmacological Basis of Therapeutics, Tenth Ed. (McGraw Hill, New York).

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as multidrug resistance. Because of the drug resistance, many cancers prove refractory to standard chemotherapeutic treatment protocols.

Patients with metastatic or refractory solid tumors face poor prognoses despite recent advances in multimodal therapy, highlighting the need for improved treatments. This persistent unmet need provides the rationale to evaluate new agents with novel mechanisms of action. A significant number of patients fail to respond to standard-of-care chemotherapy and chemoimmunotherapy combinations. Exclusion and functional suppression of immune effector cells from the TME is a critical determinant of resistance to these standard therapies.

The c-Jun N-terminal kinases (JNKs) are mitogen-activated protein kinases that consist of 3 family members: JNK1, JNK2, and JNK3. In cancer, JNK1/2 drive an immune-suppressive and chemotherapy-resistant tumor microenvironment (TME). In humans, JNK pathway activation is associated with increased stroma, chemotherapy resistance, reduced T-cell function, and poor outcome in pancreatic, breast, lung, colorectal, and other cancers (Insua-Rodriguez J, et al. Stress signaling in breast cancer cells induces matrix components that promote chemoresistant metastasis. EMBO Mol Med 2018; 10:e9003; Semba T, et al. Identification of the JNK-active triple-negative breast cancer cluster associated with an immunosuppressive tumor microenvironment. J Natl Cancer Inst 2022; 114:97-108; Lisanti M P, et al. JNK1 stress signaling is hyper-activated in high breast density and the tumor stroma: connecting fibrosis, inflammation, and stemness for cancer prevention. Cell Cycle 2014; 13:580-99; Lipner M B, et al. Irreversible JNK1-JUN inhibition by JNK-IN-8 sensitizes pancreatic cancer to 5-FU/FOLFOX chemotherapy. JCI Insight 2020; 5:e129905; Vasilevskaya I A et al. JNK1 inhibition attenuates hypoxiainduced authophagy and sensitizes to chemotherapy. Mol Cancer Res 2016; 14:753-63; Xu L et al. Cisplatin induces expression of drug resistance-related genes through c-jun N-terminal kinase pathway in human lung cancer cells. Cancer Chemother Pharmacol 2017; 80:235-42). Alone and in combination with chemotherapy, pharmacologic inhibition of JNK reduced tumor growth in breast and pancreatic models, and INK inhibitor treatment resulted in enhanced intratumoral cluster of differentiation 8 (CD8) T-cell numbers and activity in part by reducing chemokine (C—C motif) ligand 2 (CCL2) production in macrophages (Semba T, et al. 2022; Sato T, et al. c-Jun N-terminal kinase in pancreatic tumor stroma augments tumor development in mice. Cancer Sci 2017; 108:2156-65). The JNK pathway has emerged as a potential driver of resistance to immunotherapy (IO) by generating an immunosuppressive and immunoexclusive TME. Published non-clinical data support a role for JNK in promoting immune evasion of tumor cells and modulating the TME (Dong C, et al., Signaling by the JNK group of MAP kinases. c-jun N-terminal, Kinase. J Clin Immunol 2001; 21:253-7).

The compound chemically named 2-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), and tautomers thereof are disclosed in U.S. Pat. No. 9,139,534, and International Pub. No. WO2012/145569, the entireties of each of which are incorporated by reference herein. Formulations of 2 (tert-butylamino)-4-((1R,3R,4R)-3-hydroxy-4-methylcyclohexylamino)-pyrimidine-5-carboxamide (“Compound 1”), or a pharmaceutically acceptable salt, stereoisomer, tautomer, solid form, polymorph, hydrate, clathrate, or solvate thereof (collectively referred to herein as “Compound A”), are disclosed in U.S. Pat. No. 9,796,685, and International Pub. No. WO2016/100310, the entireties of each of which are incorporated by reference herein.

Compound 1 is a JNK (c-Jun N-terminal kinases) inhibitor that has demonstrated reduction in fibrotic biomarkers such as Tenascin C (TNC). In cancer, elevated fibrotic biomarkers like propeptides of type III collagen (PRO-C3), propeptides of type VI collagen (PRO-C6), and TNC predict poor survival (e.g, in non-small cell lung cancer (NSCLC), metastatic triple negative breast cancer (mTNBC), colorectal carcinoma (CRC), pancreatic adenocarcinoma (PAAD), and melanoma) (Giussani M, et al. Tumor extracellular matrix remodeling: new perspectives as a circulating tool in the diagnosis and prognosis of solid tumors. Cells 2019; 8:81; Willumsen N, et al. Collagen fragments quantified in serum as measures of desmoplasia associate with survival outcome in patients with advanced pancreatic cancer. Sci Rep 2019; 9:19761; Nissen N I, et al. Prognostic value of blood-based fibrosis biomarkers in patients with metastatic colorectal cancer receiving chemotherapy and bevacizumab. Sci Rep 2021; 11:865; Jensen C, et al. Non-invasive biomarkers derived from the extracellular matrix associate with response to immune checkpoint blockade (anti-CTLA-4) in metastatic melanoma patients. J Immunother Cancer 2018; 6:152).

Accordingly, compounds that can inhibit JNK pathway activation may be useful alone or in combination therapy for the treatment and prevention of various forms of cancer.

### SUMMARY

Provided herein are methods of treating and managing cancer, including newly diagnosed cancer, as well as cancer that is relapsed, refractory or resistant to conventional chemotherapy, which comprise administering to a patient having said cancer a therapeutically or prophylactically effective amount of 2-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), having the following structure:

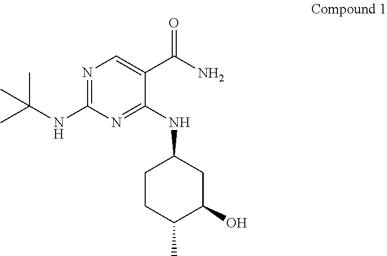 [SMILES: C[C@@H]1CC[C@@H](Nc2nc(NC(C)(C)C)ncc2C(N)=O)C[C@H]1O]

or a pharmaceutically acceptable salt, stereoisomer, tautomer, solid form, polymorph, hydrate, clathrate, or solvate thereof (collectively referred to herein as “Compound A”). Provided herein are methods of treating and managing cancer, including newly diagnosed cancer, as well as cancer that is relapsed, refractory or resistant to conventional chemotherapy, which comprise administering to a patient having said cancer (i) a therapeutically or prophylactically effective amount of 2-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) and (ii) chemotherapy. Provided herein is 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) for use in such methods of treating and managing cancer, including newly diagnosed cancer, as well as cancer that is relapsed, refractory or resistant to conventional chemotherapy.

Provided herein are methods of treating and managing cancer, including newly diagnosed cancer, as well as cancer that is relapsed, refractory or resistant to conventional chemotherapy, which comprise administering to a patient having said cancer (i) a therapeutically or prophylactically effective amount of 2-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), having the following structure:

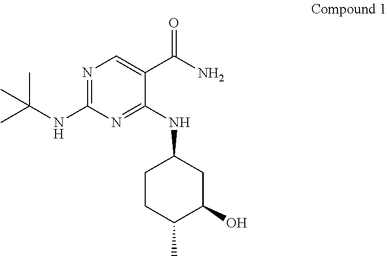 [SMILES: C[C@@H]1CC[C@@H](Nc2nc(NC(C)(C)C)ncc2C(N)=O)C[C@H]1O]

or a pharmaceutically acceptable salt, stereoisomer, tautomer, solid form, polymorph, hydrate, clathrate, or solvate thereof (collectively referred to herein as “Compound A”), and (ii) nivolumab. Provided herein is 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) for use in such methods of treating and managing cancer, including newly diagnosed cancer, as well as cancer that is relapsed, refractory or resistant to conventional chemotherapy.

Also provided herein are methods of managing cancer (e.g., preventing its recurrence, or lengthening the time of remission), which comprise administering to a patient in need of such management a therapeutically or prophylactically effective amount of (i) 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”), and (ii) chemotherapy. Provided herein is (i) 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) for use in such methods of managing cancer (e.g., preventing its recurrence, or lengthening the time of remission).

Further provided herein are methods of managing cancer (e.g., preventing its recurrence, or lengthening the time of remission), which comprise administering to a patient in need of such management a therapeutically or prophylactically effective amount of (i) 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”); and (ii) nivolumab. Provided herein is (i) 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) for use in such methods of managing cancer (e.g., preventing its recurrence, or lengthening the time of remission).

In certain embodiments, provided herein are methods for the treatment or management of cancer. In one embodiment, the cancer is a solid tumor (e.g., lung cancer, breast cancer, pancreatic cancer, renal cell carcinoma, colorectal carcinoma, squamous cell carcinoma, or sarcoma).

In one embodiment, the solid tumor is an advanced solid tumor. In one embodiment, the solid tumor is metastatic, relapsed and/or refractory. In one embodiment, the solid tumor is resistant to conventional chemotherapy. In one embodiment, the solid tumor is resistant to immunotherapy (IO).

The embodiments provided herein encompass methods for screening or identifying cancer patients, e.g., patients having lung cancer, breast cancer, squamous cell carcinoma of head and neck, pancreatic adenocarcinoma, pancreatic ductal adenocarcinoma, renal cell carcinoma, colorectal carcinoma, or sarcoma, for treatment with 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”). Provided herein are methods for screening or identifying cancer patients, e.g., lung cancer, breast cancer, squamous cell carcinoma of head and neck, pancreatic adenocarcinoma, pancreatic ductal adenocarcinoma, renal cell carcinoma, colorectal carcinoma, or sarcoma, for treatment with (i) 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) in combination with chemotherapy.

The embodiments provided herein encompass methods for screening or identifying cancer patients, e.g., lung cancer, breast cancer, squamous cell carcinoma of head and neck, pancreatic adenocarcinoma, pancreatic ductal adenocarcinoma, renal cell carcinoma, colorectal carcinoma, or sarcoma, for treatment with 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) in combination with nivolumab.

In one embodiment, provided herein is a method for treating or managing a solid tumor, comprising:
(i) identifying a patient having a solid tumor sensitive to treatment with Compound 1, or a pharmaceutically acceptable salt, stereoisomer, tautomer, solid form, polymorph, hydrate, clathrate, or solvate thereof; and
(ii) administering to the patient a therapeutically effective amount of Compound 1, or a pharmaceutically acceptable salt, stereoisomer, tautomer, solid form, polymorph, hydrate, clathrate, or solvate thereof (collectively referred to herein as “Compound A”). Provided herein is 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) for use in such a method for treating or managing a solid tumor.

In one embodiment, provided herein is a method for treating or managing a solid tumor, comprising:
(i) identifying a patient having a solid tumor sensitive to treatment with (a) Compound 1, or a pharmaceutically acceptable salt, stereoisomer, tautomer, solid form, polymorph, hydrate, clathrate, or solvate thereof (collectively referred to herein as “Compound A”) and (b) nivolumab; and
(ii) administering to the patient a therapeutically effective amount of (a) Compound 1, or or a pharmaceutically acceptable salt, stereoisomer, tautomer, solid form, polymorph, hydrate, clathrate, or solvate thereof (collectively referred to herein as “Compound A”) and (b) nivolumab. Provided herein is 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) for use in such a method for treating or managing a solid tumor.

In one embodiment, provided herein is a method for treating or managing a solid tumor, comprising:
(i) identifying a patient having a solid tumor sensitive to treatment with (a) Compound 1, or a pharmaceutically acceptable salt, stereoisomer, tautomer, solid form, polymorph, hydrate, clathrate, or solvate thereof and (b) a chemotherapy; and
(ii) administering to the patient a therapeutically effective amount of (a) Compound 1, or or a pharmaceutically acceptable salt, stereoisomer, tautomer, solid form, polymorph, hydrate, clathrate, or solvate thereof and (b) a chemotherapy. Provided herein is 3-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), or a pharmaceutically acceptable salt, stereoisomer, tautomer, solid form, polymorph, hydrate, clathrate, or solvate thereof (collectively referred to herein as “Compound A”) for use in such a method for treating or managing a solid tumor.

In one embodiment, provided herein are pharmaceutical compositions comprising an effective amount of Compound A as described herein, and a pharmaceutically acceptable carrier, excipient or vehicle. In some embodiments the pharmaceutical composition is suitable for oral, parenteral, mucosal, transdermal or topical administration.

Also provided herein are pharmaceutical compositions comprising (i) about 200 to about 800 mg of Compound A as described herein, and (ii) about 300 to about 600 mg of nivolumab. Also provided herein are pharmaceutical compositions comprising (i) about 900 mg of Compound A as described herein, and (ii) about 300 to about 600 mg of nivolumab.

Also provided herein are pharmaceutical compositions comprising (i) about 200 to about 800 mg of Compound A as described herein, and (ii) about 50 to about 100 mg/m² of docetaxel or a pharmaceutically acceptable salt, stereoisomer, tautomer, solid form, polymorph, hydrate, clathrate, or solvate thereof. Also provided herein are pharmaceutical compositions comprising (i) about 900 mg of Compound A as described herein and (ii) about 50 to about 100 mg/m² of docetaxel or a pharmaceutically acceptable salt, stereoisomer, tautomer, solid form, polymorph, hydrate, clathrate, or solvate thereof.

Also provided herein are pharmaceutical compositions comprising (i) about 200 to about 800 mg of Compound A as described herein, and (ii) about 300 to about 2000 mg/m² of capecitabine or a pharmaceutically acceptable salt, stereoisomer, tautomer, solid form, polymorph, hydrate, clathrate, or solvate thereof. Also provided herein are pharmaceutical compositions comprising (i) about 900 mg of Compound A as described herein and (ii) about 300 to about 2000 mg/m² of capecitabine or a pharmaceutically acceptable salt, stereoisomer, tautomer, solid form, polymorph, hydrate, clathrate, or solvate thereof. Also provided herein are pharmaceutical compositions comprising (i) about 200 to about 800 mg of Compound A as described herein, and (ii) about 50 mg/m² to about 200 mg/m² per day of paclitaxel or a pharmaceutically acceptable salt, stereoisomer, tautomer, solid form, polymorph, hydrate, clathrate, or solvate thereof. Also provided herein are pharmaceutical compositions comprising (i) about 900 mg of Compound A as described herein and (ii) about 50 mg/m² to about 200 mg/m² per day of paclitaxel or a pharmaceutically acceptable salt, stereoisomer, tautomer, solid form, polymorph, hydrate, clathrate, or solvate thereof.

Also provided herein are pharmaceutical compositions comprising (i) about 200 to about 800 mg of Compound A as described herein, and (ii) about 200 mg/m² to about 1,000 mg/m² per day of fluorouracil (5-FU) or a pharmaceutically acceptable salt, stereoisomer, tautomer, solid form, polymorph, hydrate, clathrate, or solvate thereof. Also provided herein are pharmaceutical compositions comprising (i) about 900 mg of Compound A as described herein and (ii) about 200 mg/m² to about 1,000 mg/m² per day of fluorouracil (5-FU) or a pharmaceutically acceptable salt, stereoisomer, tautomer, solid form, polymorph, hydrate, clathrate, or solvate thereof.

Further provided herein are methods for using the pharmaceutical compositions, and dosage forms of Compound A for treating and managing solid tumors, as described herein.

Also provided herein is a pharmaceutical composition of Compound A for use in a method for treating and managing solid tumors, as described herein. Also provided herein is a dosage form of Compound A for use in a method for treating and managing solid tumors, as described herein.

Also provided herein is a kit comprising a pharmaceutical composition comprising (i) Compound A as described herein; and (ii) nivolumab. In one embodiment, the kit comprises (i) about 200 to about 800 mg of Compound A as described herein, and (ii) about 300 to about 600 mg of nivolumab. In one embodiment, the kit comprises (i) about 200 to about 800 mg of Compound A as described herein, and (ii) about 900 mg of nivolumab. In one embodiment, the kit comprises (i) about 200 to about 900 mg of Compound A as described herein, and (ii) about 900 mg of nivolumab.

Also provided herein is a kit comprising a pharmaceutical composition comprising (i) Compound A as described herein; and (ii) docetaxel or a pharmaceutically acceptable salt, stereoisomer, tautomer, solid form, polymorph, hydrate, clathrate, or solvate thereof. In one embodiment, the kit comprises (i) about 200 to about 800 mg of Compound A as described herein, and (ii) about 50 to about 100 mg/m² of docetaxel or a pharmaceutically acceptable salt, stereoisomer, tautomer, solid form, polymorph, hydrate, clathrate, or solvate thereof. In one embodiment, the kit comprises (i) 900 mg of Compound A as described herein, and (ii) about 50 to about 100 mg/m² of docetaxel or a pharmaceutically acceptable salt, stereoisomer, tautomer, solid form, polymorph, hydrate, clathrate, or solvate thereof.

Also provided herein is a kit comprising a pharmaceutical composition comprising (i) Compound A as described herein; and (ii) capecitabine or a pharmaceutically acceptable salt, stereoisomer, tautomer, solid form, polymorph, hydrate, clathrate, or solvate thereof. In one embodiment, the kit comprises (i) about 200 to about 800 mg of Compound A as described herein, and (ii) about 300 to about 2000 mg/m² of capecitabine or a pharmaceutically acceptable salt, stereoisomer, tautomer, solid form, polymorph, hydrate, clathrate, or solvate thereof. In one embodiment, the kit comprises (i) about 900 mg of Compound A as described herein, and (ii) about 300 to about 2000 mg/m² of capecitabine or a pharmaceutically acceptable salt, stereoisomer, tautomer, solid form, polymorph, hydrate, clathrate, or solvate thereof.

Also provided herein is a kit comprising a pharmaceutical composition comprising (i) Compound A as described herein; and (ii) paclitaxel or a pharmaceutically acceptable salt, stereoisomer, tautomer, solid form, polymorph, hydrate, clathrate, or solvate thereof. In one embodiment, the kit comprises (i) about 200 to about 800 mg of Compound A as described herein, and (ii) about 50 mg/m² to about 200 mg/m² of paclitaxel or a pharmaceutically acceptable salt, stereoisomer, tautomer, solid form, polymorph, hydrate, clathrate, or solvate thereof. In one embodiment, the kit comprises (i) about 900 mg of Compound A as described herein, and (ii) about 50 mg/m² to about 200 mg/m² of paclitaxel or a pharmaceutically acceptable salt, stereoisomer, tautomer, solid form, polymorph, hydrate, clathrate, or solvate thereof.

Also provided herein is a kit comprising a pharmaceutical composition comprising (i) Compound A as described herein; and (ii) fluorouracil (5-FU) or a pharmaceutically acceptable salt, stereoisomer, tautomer, solid form, polymorph, hydrate, clathrate, or solvate thereof. Also provided herein is a kit comprising a pharmaceutical composition comprising (i) Compound A as described herein; and (ii) fluorouracil (5-FU) or a pharmaceutically acceptable salt, stereoisomer, tautomer, solid form, polymorph, hydrate, clathrate, or solvate thereof. In one embodiment, the kit comprises (i) about 200 to about 800 mg of Compound A as described herein, and (ii) and about 200 mg/m² to about 1,000 mg/m² per day of fluorouracil (5-FU) or a pharmaceutically acceptable salt, stereoisomer, tautomer, solid form, polymorph, hydrate, clathrate, or solvate thereof. In one embodiment, the kit comprises (i) about 900 mg of Compound A as described herein, and (ii) about 200 mg/m² to about 1,000 mg/m² per day of fluorouracil (5-FU) or a pharmaceutically acceptable salt, stereoisomer, tautomer, solid form, polymorph, hydrate, clathrate, or solvate thereof.

### BRIEF DESCRIPTION OF THE FIGURES

FIG. 1 illustrates the Study Design, Part 1. Abbreviations used are as follows: IO, immunotherapy; MSS CRC, microsatellite-stable colorectal carcinoma; mTNBC, metastatic triple negative breast cancer; n, number of participants; NSCLC, non-small cell lung cancer; PAAD, pancreatic adenocarcinoma; QD, daily; RCC, renal cell carcinoma; SCCHN, squamous cell carcinoma of head and neck.

FIG. 2 illustrates the Study Design, Part 2 and Part 2A Abbreviations used are as follows: approx., approximately; DL1, dose level 1; DL2, dose level 2; Doce, docetaxel; IO, immunotherapy; mTNBC, metastatic triple negative breast cancer; n, number of participants; Nivo, nivolumab; NSCLC, non-small cell lung cancer; Q3W, every 3 weeks; QD, daily; R, randomization; TBD, to be determined.

FIG. 3 illustrates the Study Design, Part 2B. Abbreviations used are as follows: 2L+, second line or later; BID, twice daily; Cape, capecitabine; DL1, dose level 1; DL2, dose level 2; mTNBC, metastatic triple negative breast cancer; TBD, to be determined.

FIGS. 4A-4E illustrate the effects of Compound 1 on the Potency of Paclitaxel in a 2D Cell Line Viability Assay. Graph represents the chemotherapy dose response curve on 2D cellular confluence 4 days after paclitaxel treatment in the presence of DMSO, 5 μM Compound 1 or 20 μM Compound 1. Data points indicate mean and Standard Deviation of 2 biological replicates. Graphs are representative of at least 2 independent experiments. FIG. 4A shows results in 4T1.2 cell line. FIG. 4B shows results in A549 cell line. FIG. 4C shows results in EMT6 cell line. FIG. 4D shows results in NHI-H460 cell line. FIG. 4E shows results in HT29 cell line. Abbreviations: DMSO, dimethylsulfoxide.

FIGS. 5A-5E illustrate the chemotherapy dose response curve on 2D cellular confluence 4 days after 5-FU treatment in the presence of DMSO, 5 μM Compound 1 or 20 μM Compound 1. Data points indicate mean and Standard Deviation of 2 biological replicates. Graphs are representative of at least 2 independent experiments. FIG. 5A shows results in 4T1.2 cell line.

FIG. 5B shows results in A549 cell line. FIG. 5C shows results in EMT6 cell line. FIG. 5D shows results in NHI-H460 cell line. FIG. 5E shows results in HT29 cell line. Abbreviation: 5-FU, 5-Fluorouracil; DMSO, dimethylsulfoxide.

FIGS. 6A-6H illustrate the dose response of chemotherapy combinations Compound 1 in patient derived organoids. Chemotherapy dose response curve on 3D organoid colony formation 8 to 13 days after indicated chemotherapy treatment in the presence of DMSO, M Compound 1 or 20 μM Compound 1. Data points indicate mean and standard deviation of 2 biological replicates. These data are representative of the data quantified in Table 5. Abbreviations: Conc, concentration; DMSO, dimethylsulfoxide; Std Dev, standard deviation. FIG. 6A shows the combination activity with paclitaxel in MAXFT2990. FIG. 6B shows the combination activity with SN-38 in MAXFT2990. FIG. 6C shows the combination activity with paclitaxel in PAXF1657. FIG. 6D shows the combination activity with gemcitabine in LXFE2478. FIG. 6E shows the combination activity with paclitaxel in LXFE2478. FIG. 6F shows the combination activity with paclitaxel in LXFE690. FIG. 6G shows the combination activity with SN-38 in CXF1256. FIG. 6H shows the combination activity with carboplatin in LXFA644.

FIG. 7 illustrates the levels of phospho-c-Jun in NHLF and non-small cell lung CAF in response to TGF-β in the presence of DMSO or varying concentrations of Compound 1. Normal human lung fibroblasts (NHLF, black circle) or lung cancer-associated fibroblasts (CAF, black square) were treated with 10 ng/mL TGF-β after 1 hour pre-incubation with a range of Compound 1 concentrations, and cells were lysed 2 hours later. Phospho-c-Jun levels were evaluated by western blot. Abbreviations: CAF, cancer-associated fibroblasts; NHLF, normal lung human fibroblasts; TGF-β, transforming growth factor beta.

FIGS. 8A-8C illustrate the effect of Compound 1 on Transcriptional Markers of Fibroblast Activation in Normal Fibroblasts Treated with TGF-β. FIG. 8A shows effects on TNC. FIG. 8B shows effects on ACTA2. FIG. 8C shows effects on PAI-1. Normal human lung fibroblasts (NHLF, black bars) were treated with 10 ng/mL TGF-β after 1 hour pre-incubation with a range of Compound 1 concentrations, and cells were lysed 24 hours later. Indicated gene transcripts were assessed by RT-PCR relative to GAPDH. Data were normalized to expression after TGF-β treatment in the presence of only DMSO. Data are presented as mean of 3 biological replicates, and error bars indicate standard deviation. The graphs are representative of at least 2 independent experiments. *P<0.05; ** P<0.01, ***P<0.001; ****P<0.0001 by student's t-test. Abbreviations: ACTA2, smooth muscle actin; CAF, cancer associated fibroblasts; DMSO, dimethyl sulfoxide; GAPDH, glyceraldehyde-3-phosphate dehydrogenase; NHLF, normal lung human fibroblasts; PAI-1, plasminogen activator inhibitor 1; RNA, ribonucleic acid; RT-PCR, reverse transcription polymerase chain reaction; TGF-β, transforming growth factor beta; TNC, tenascin C.

FIGS. 9A-9C illustrates the effect of Compound 1 on Transcriptional Markers of Fibroblast Activation in Lung Cancer Associated Fibroblasts Treated with TGF-β. FIG. 9A shows effects on TNC. FIG. 9B shows effects on ACTA2. FIG. 9C shows effects on PAI-1. Lung cancer-associated fibroblasts (CAF, black bars) were treated with 10 ng/mL TGF-β after 1-hour pre-incubation with a range of Compound 1 concentrations, and cells were lysed 24 hours later. Indicated gene transcripts were assessed by RT-PCR relative to GAPDH. Data were normalized to expression after TGF-β-treatment in the presence of only DMSO. Data are presented as mean of 3 biological replicates, and error bars indicate standard deviation. The graphs are representative of at least 2 independent experiments. *P<0.05; ** P<0.01, ***P<0.001; ****P<0.0001 by student's t-test. Abbreviations: ACTA2, smooth muscle actin; CAF, cancer associated fibroblasts; DMSO, dimethyl sulfoxide; GAPDH, glyceraldehyde-3-phosphate dehydrogenase; NHLF, normal lung human fibroblasts; PAI-1, plasminogen activator inhibitor 1; RNA, ribonucleic acid; RT-PCR, reverse transcription polymerase chain reaction; TGF-0, transforming growth factor beta; TNC, tenascin C.

FIGS. 10A-10D illustrate the effect of Compound 1 on TGF-β-induced transcripts in A549 tumor cells. FIG. 10A shows effects in COL1A1. FIG. 10B shows effects in PAI-1.

FIG. 10C shows effects in TNC. FIG. 10D shows effects in LRRC15. A549 cells were treated with 10 ng/mL TGF-β after 1 hour pre-incubation with a range of Compound 1 concentrations, and cells were lysed 24 hours later. Indicated gene transcripts were assessed by RT-PCR relative to GAPDH. Data were normalized to expression after TGF-β-treatment in the presence of only DMSO. Data are presented as mean of 3 biological replicates, and error bars indicate standard deviation. The graphs are representative of at least 2 independent experiments. *P<0.05; ** P<0.01, ***P<0.001; ****P<0.0001 by student's t-test. Abbreviations: CAF, cancer-associated fibroblasts; COL1A1, collagen 1 alpha 1; DMSO, dimethyl sulfoxide; GAPDH, glyceraldehyde-3-phosphate dehydrogenase; LRRC15, leucine-rich repeat containing 15; NHLF, normal lung human fibroblasts; ns, not significant; PAI-1, plasminogen activator inhibitor 1; RNA, ribonucleic acid; RT-PCR, reverse transcription polymerase chain reaction; TGF-0, transforming growth factor beta; TNC, tenascin C.

FIG. 11 illustrates the effect of Compound 1 Treatment on CCL2 Secretion in Primary Human Macrophages. Primary human monocytes were differentiated into macrophages and treated with varying doses of DMSO or Compound 1. The CCL2 levels were measured in the cell supernatant by ELISA 24 hours after drug treatment. The CCL2 secretion levels were normalized to the equivalent DMSO concentration. Data are plotted as the mean and standard deviation of 3 donors and representative of 2 independent experiments. *P<0.05; ** P<0.01, ***P<0.001; ****P<0.0001 by student's t-test. Abbreviations: CCL2, C—C motif chemokine ligand 2; DMSO, dimethyl sulfoxide; ELISA, enzyme-linked immunosorbent assay.

FIG. 12 illustrates the effect of Compound 1 on Primary Macrophage Viability. Primary human monocytes were differentiated into macrophages and treated with varying doses of DMSO or Compound 1. Cell viability was assessed by Cell Titer Glo 24 hours after drug treatment. Viability in drug treatment groups was normalized to the equivalent DMSO concentration. Data are plotted as the mean and standard deviation of 3 donors and representative of 2 independent experiments. Abbreviations: DMSO, dimethyl sulfoxide.

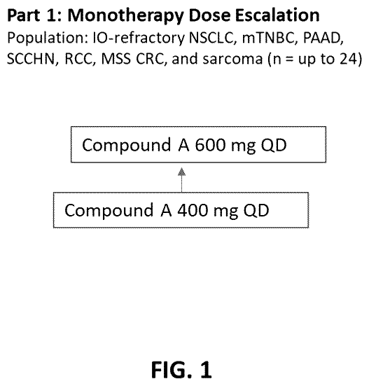

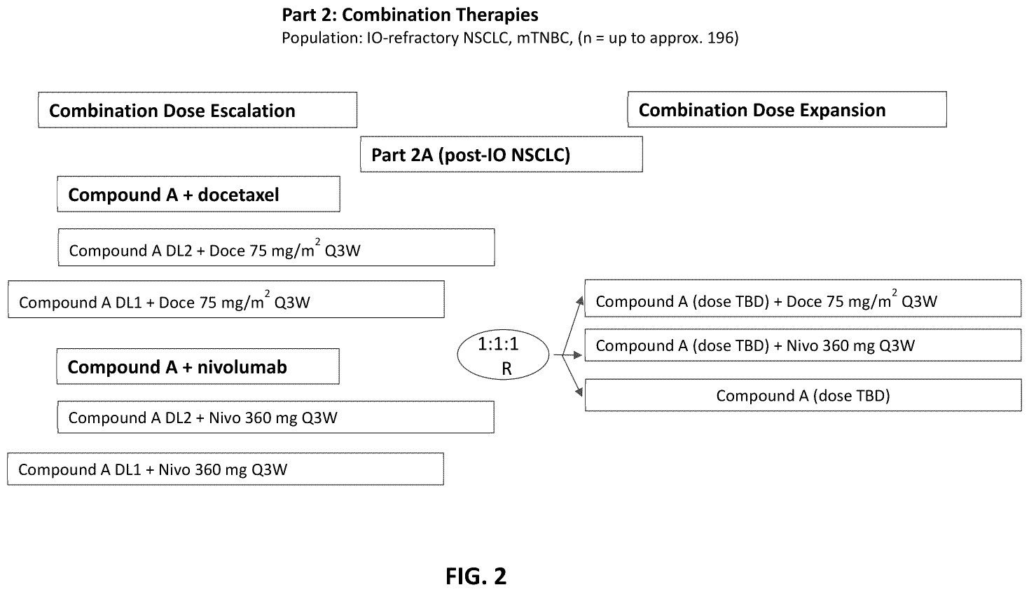

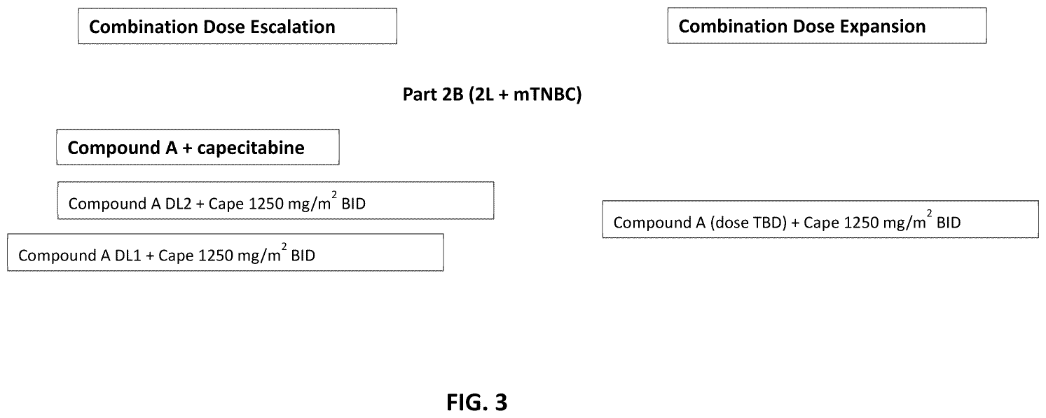

### DETAILED DESCRIPTION

### Definitions

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of the compounds provided herein, with or without other additional active agent, after the onset of symptoms of the particular disease.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of the compounds provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease, or lengthening the time during which the disease remains in remission.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

Combination therapy or “in combination with” refer to the use of more than one therapeutic agent to treat a particular disorder or condition. By “in combination with,” it is not intended to imply that the therapeutic agents must be administered at the same time and/or formulated for delivery together, although these methods of delivery are within the scope of this disclosure. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

As used herein, the terms “additional active agent,” “active agent” and “active ingredient” refer to pharmacologically active compounds useful in the treatment of particular types of cancer, and certain diseases and conditions associated with or characterized by undesired angiogenesis. The active agents can be large molecules (e.g., proteins) or small molecules (e.g., synthetic inorganic, organometallic, or organic molecules). Examples of large molecule active agents include, but are not limited to, hematopoietic growth factors, cytokines, and monoclonal and polyclonal antibodies. In certain embodiments, large molecule active agents are biological molecules, such as naturally occurring or artificially made proteins. Proteins that are particularly useful in this disclosure include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; GC-CSF, BCG, cancer antibodies, and EPO. Active agents that are small molecules can also be used to alleviate adverse effects associated with the administration of the compounds provided herein. However, like some large molecules, many are believed to be capable of providing a synergistic effect when administered with (e.g., before, after or simultaneously) the compounds provided herein. Examples of small molecule additional active agents include, but are not limited to, anti-cancer agents, antibiotics, immunosuppressive agents, and steroids.

In certain embodiments, the active agent is at least one chemotherapeutic agent, at least one anti-inflammatory agent, or at least one immunosuppressive and/or immunomodulatory agent. In one embodiment, such a chemotherapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine, cladribine, capecitabine and similar agents. In one embodiment, such a chemotherapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin, oxaliplatin and other platinum derivatives, such as carboplatin, and similar agents. In one embodiment, such a chemotherapeutic agent may be selected from an antibiotic, such as dactinomycin (formerly actinomycin), bleomycin, daunorubicin (formerly daunomycin), idarubicin, mithramycin, mitomycin, mitoxantrone, plicamycin, anthramycin (AMC) and similar agents. In one embodiment, such a chemotherapeutic agent may be selected from an anti-mitotic agent, such as taxanes, for instance docetaxel, and paclitaxel. In one embodiment, such a chemotherapeutic agent may be selected from a topoisomerase inhibitor, such as topotecan or SN-38. In one embodiment, such a chemotherapeutic agent may be selected from a growth factor inhibitor, such as an inhibitor of ErbB1 (EGFR) (such as gefitinib (Iressa®), cetuximab (Erbitux®), erlotinib (Tarceva®), 2F8 (disclosed in WO 2002/100348) and similar agents), an inhibitor of ErbB2 (Her2/neu) (such as trastuzumab (Herceptin®) and similar agents) and similar agents. In one embodiment, such a growth factor inhibitor may be a farnesyl transferase inhibitor, such as SCH-66336 and RI 15777. In one, embodiment, such a growth factor inhibitor may be a vascular endothelial growth factor (VEGF) inhibitor, such as bevacizumab (Avastin®). In one embodiment, such a chemotherapeutic agent may be a tyrosine kinase inhibitor, such as imatinib (Glivec, Gleevec STI571), lapatinib, PTK787/ZK222584 and similar agents. In one embodiment, such a chemotherapeutic agent may be a histone deacetylase inhibitor. Examples of such histone deacetylase inhibitors include hydroxamic acid-based hybrid polar compounds, such as SAHA (suberoylanilide hydroxamic acid). In one embodiment, such a chemotherapeutic agent may be a P38a MAP kinase inhibitor, such as SCIO-469.

In a further embodiment, the combination therapy of the invention further includes administration of at least one anti-inflammatory agent. In one embodiment such an anti-inflammatory agent may be selected from a steroidal drug and a NSAID (nonsteroidal anti-inflammatory drug). In one embodiment such an anti-inflammatory agent may be selected from aspirin and other salicylates, Cox-2 inhibitors (such as rofecoxib and celecoxib), NSAIDs (such as ibuprofen, fenoprofen, naproxen, sulindac, diclofenac, piroxicam, ketoprofen, diflunisal, nabumetone, etodolac, oxaprozin, and indomethacin), anti-IL6R antibodies, anti-IL8 antibodies (e.g. 10F8 described in WO2004/058797), anti-IL15 antibodies, anti-IL15R antibodies, anti-CD4 antibodies, anti-CD11a antibodies (e.g., efalizumab), anti-alpha-4/beta-1 integrin (VLA4) antibodies (e.g natalizumab), CTLA4-Ig for the treatment of inflammatory diseases, prednisolone, prednisone, disease modifying antirheumatic drugs (DMARDs) such as methotrexate, hydroxychloroquine, sulfasalazine, pyrimidine synthesis inhibitors (such as leflunomide), IL-1 receptor blocking agents (such as anakinra), TNF-α blocking agents (such as etanercept, infliximab, and adalimumab) and similar agents.

In a further embodiment, the combination therapy of the invention further includes administration of at least one immunosuppressive and/or immunomodulatory agent to a subject in need thereof. In one embodiment, such an immunosuppressive and/or immunomodulatory agent may be selected from cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, F L, 2004).

As used herein, the terms “patient” or “subject” is defined herein to include animals, such as mammals, including, but not limited to, primates (e.g., humans), cows, sheep, goats, horses, dogs, cats, rabbits, rats, mice, monkeys, chickens, turkeys, quails, or guinea pigs and the like, in one embodiment a subject is a mammal, in another embodiment a subject is a human. In one embodiment, a subject is a human having or at risk for having a solid tumor. In another embodiment, a subject is a human having or at risk for having a solid tumor treatable or manageable by inhibition of a INK pathway, or a symptom thereof. In one embodiment, a subject is fasted. In another embodiment, a subject is fed.

As used herein, and unless otherwise specified, the term “tumor” refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

As used herein, and unless otherwise specified, the term “relapsed” refers to a situation where a subject or a mammal, which has had a remission of cancer after therapy has a return of cancer cells.

As used herein, and unless otherwise specified, an “effective patient tumor response” refers to any increase in the therapeutic benefit to the patient. An “effective patient tumor response” can be, for example, a 5%, 10%, 25%, 50%, or 100% decrease in the rate of progress of the tumor. An “effective patient tumor response” can be, for example, a 5%, 10%, 25%, 50%, or 100% decrease in the physical symptoms of a cancer. An “effective patient tumor response” can also be, for example, a 5%, 10%, 25%, 50%, 100%, 200%, or more increase in the response of the patient, as measured by any suitable means, such as gene expression, cell counts, assay results, etc.

As used herein, and unless otherwise specified, the term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

As used herein, and unless otherwise specified, the term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

As used herein, and unless otherwise specified, the term “monitor,” as used herein, generally refers to the overseeing, supervision, regulation, watching, tracking, or surveillance of an activity. For example, the term “monitoring the effectiveness of a compound” refers to tracking the effectiveness in treating a cancer in a patient or in a tumor cell culture. Similarly, the “monitoring,” when used in connection with patient compliance, either individually, or in a clinical trial, refers to the tracking or confirming that the patient is actually taking the immunomodulatory compound being tested as prescribed. The monitoring can be performed, for example, by following the expression of mRNA or protein biomarkers.

An improvement in the cancer or cancer-related disease can be characterized as a complete or partial response. “Complete response” (CR) refers to disappearance of all target lesions. Any pathological lymph nodes (whether target or non-target) must have reduction in short axis to about <10 mm. “Partial response” (PR) refers to at least about a 10%, 20%, 30%, 40%, 50%, 60%, 70%, 80%, or 90% decrease in all measurable tumor burden (i.e., decrease in the sum of diameters of target lesions, taking as reference the baseline sum diameters) in the absence of new lesions. The term “treatment” contemplates both a complete and a partial response. “Progressive Disease” (PD): refers to at least about a 20% increase in the sum of diameters of target lesions, taking as reference the smallest sum on study (this includes the baseline sum if that is the smallest on study). In addition to the relative increase of about 20%, the sum must also demonstrate an absolute increase of at least about 5 mm. The appearance of one or more new lesions is also considered progression. “Stable Disease” (SD) refers to neither sufficient shrinkage to qualify for PR nor sufficient increase to qualify for PD, taking as reference the smallest sum diameters while on study.

As used herein, and unless otherwise specified, the term “refractory or resistant” refers to a circumstance where a subject or a mammal, even after intensive treatment, has residual cancer cells in his body.

As used herein, and unless otherwise specified, the term “advanced” in the context of a tumor comprises, metastatic, recurrent, and/or unresectable tumors. Also the term “advanced” as used herein encompasses a measurable disease according to methods well known in the art.

As used herein, and unless otherwise specified, the term “drug resistance” refers to the condition when a disease does not respond to the treatment of a drug or drugs. Drug resistance can be either intrinsic, which means the disease has never been responsive to the drug or drugs, or it can be acquired, which means the disease ceases responding to a drug or drugs that the disease had previously responded to. In certain embodiments, drug resistance is intrinsic. In certain embodiments, the drug resistance is acquired.

As used herein, and unless otherwise specified, the term “sensitivity” and “sensitive” when made in reference to treatment with compound is a relative term which refers to the degree of effectiveness of the compound in lessening or decreasing the progress of a tumor or the disease being treated. For example, the term “increased sensitivity” when used in reference to treatment of a cell or tumor in connection with a compound refers to an increase of, at least a 5%, or more, in the effectiveness of the tumor treatment.

As used herein, and unless otherwise specified, the terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

As used herein and unless otherwise specified, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. In certain embodiments, a stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, greater than about 70% by weight, or greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### Clinical Trial Endpoints for Cancer Approval

“Overall survival” (OS) is defined as the time from first dose until death from any cause, and is measured in the intent-to-treat population. Overall survival should be evaluated in randomized controlled studies. Demonstration of a statistically significant improvement in overall survival can be considered to be clinically significant if the toxicity profile is acceptable, and has often supported new drug approval.

Several endpoints are based on cancer assessments. These endpoints include disease free survival (DFS), objective response rate (ORR), time to progression (TTP), progression-free survival (PFS), event-free survival (EFS), duration of response (DOR) and time-to-treatment failure (TTF). The collection and analysis of data on these time-dependent endpoints are based on indirect assessments, calculations, and estimates.

Generally, “disease free survival” (DFS) is defined as the time from randomization until recurrence of cancer or death from any cause. Although overall survival is a conventional endpoint for most adjuvant settings, DFS can be an important endpoint in situations where survival may be prolonged, making a survival endpoint impractical. DFS can be a surrogate for clinical benefit or it can provide direct evidence of clinical benefit. This determination is based on the magnitude of the effect, its risk-benefit relationship, and the disease setting. The definition of DFS can be complicated, particularly when deaths are noted without prior cancer progression documentation. These events can be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

“Objective response rate” (ORR) is defined as the sum of the percentage of patients who achieve complete and partial responses. Response duration usually is measured from the time of initial response until documented cancer progression. Generally, the FDA has defined ORR as the sum of partial responses plus complete responses. When defined in this manner, ORR is a direct measure of drug anticancer activity, which can be evaluated in a single-arm study. If available, standardized criteria should be used to ascertain response. A variety of response criteria have been considered appropriate (e.g., RECIST criteria) (Therasse et al., (2000) J. Natl. Cancer Inst, 92: 205-16). The significance of ORR is assessed by its magnitude and duration, and the percentage of complete responses (no detectable evidence of cancer).

“Duration of response” (DOR) is the time from achieving a response until relapse or disease progression.

“Time to progression” (TTP) and “progression-free survival” (PFS) have served as primary endpoints for drug approval. TTP is defined as the time from randomization until objective cancer progression; TTP does not include deaths. PFS is defined as the time from randomization until objective cancer progression or death. Compared with TTP, PFS is the preferred regulatory endpoint. PFS includes deaths and thus can be a better correlate to overall survival. PFS assumes patient deaths are randomly related to cancer progression. However, in situations where the majority of deaths are unrelated to cancer, TTP can be an acceptable endpoint.

As an endpoint to support drug approval, PFS can reflect cancer growth and be assessed before the determination of a survival benefit. Its determination is not confounded by subsequent therapy. For a given sample size, the magnitude of effect on PFS can be larger than the effect on overall survival. However, the formal validation of PFS as a surrogate for survival for the many different malignancies that exist can be difficult. Data are sometimes insufficient to allow a robust evaluation of the correlation between effects on survival and PFS. Cancer trials are often small, and proven survival benefits of existing drugs are generally modest. The role of PFS as an endpoint to support licensing approval varies in different cancer settings. Whether an improvement in PFS represents a direct clinical benefit or a surrogate for clinical benefit depends on the magnitude of the effect and the risk-benefit of the new treatment compared to available therapies.

“Event-free survival” (EFS) is the time from study entry until any treatment failure, including disease progression, treatment discontinuation for any reason, or death.

“Time-to-treatment failure” (TTF) is defined as a composite endpoint measuring time from randomization to discontinuation of treatment for any reason, including disease progression, treatment toxicity, and death. TTF is not recommended as a regulatory endpoint for drug approval. TTF does not adequately distinguish efficacy from these additional variables. A regulatory endpoint should clearly distinguish the efficacy of the drug from toxicity, patient or physician withdrawal, or patient intolerance.

In certain embodiments, the methods provided herein are useful for achieving one or more of these clinical trial endpoints in a patient. In certain embodiments, the methods provided herein are useful for improving one or more of these clinical trial endpoints in a patient.

### Compounds

In certain embodiments, the compound for use in the compositions and methods provided herein is 2-(tert-butylamino)-4-((1R,3R,4R)-3-hydroxy-4-methylcyclohexylamino)-pyrimidine-5-carboxamide (alternatively named 2-[(1,1-dimethylethyl)amino]-4-[[(1R,3R,4R)-3-hydroxy-4-methylcyclohexyl]amino]-5-pyrimidinecarboxamide) (“Compound 1”), having the following structure:

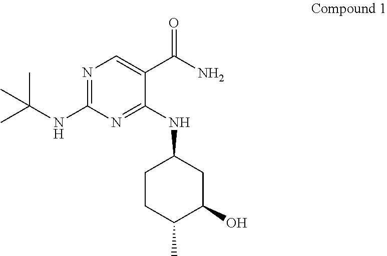 [SMILES: C[C@@H]1CC[C@@H](Nc2nc(NC(C)(C)C)ncc2C(N)=O)C[C@H]1O]

or a pharmaceutically acceptable salt, stereoisomer, tautomer, solid form, polymorph, hydrate, clathrate, or solvate thereof (collectively referred to herein as “Compound A”).

Compound 1 can be prepared according to the methods described in U.S. Pat. No. 9,139,534, and International Pub. No. WO2012/145569; the entireties of each of which are incorporated by reference herein. Formulations of Compound A are disclosed in U.S. Pat. No. 9,796,685, and International Pub. No. WO2016/100310, the entirety of each of which is incorporated herein by reference. The compound can be also synthesized according to other methods apparent to those of skill in the art based upon the teaching of these publications.

Compounds provided herein markedly inhibit the JNK family. In cancer, JNK1/2 drive an immune-suppressive and chemotherapy-resistant tumor microenvironment (TME). In humans, JNK pathway activation is associated with increased stroma, chemotherapy resistance, reduced T-cell function, and poor outcome in pancreatic, breast, lung, colorectal carcinoma, and other cancers. Without being limited by theory, one of the biological effects exerted by the compounds provided herein is the reduction in fibrotic biomarkers such as Tenascin C (TNC). The compounds provided herein inhibit INK patway activation leading to an immune-mediated decrease in tumor growth. The compounds provided herein have the capability to enhance the response to chemotherapy or nivolumab in patients with advanced solid tumors.

In certain embodiments, without being limited by theory, the biological effects exerted by the compounds provided herein include, but are not limited to, enhancing the activity of chemotherapy or immunotherapy (IO) to increase response rates and durability of response in patients with solid tumors. In one embodiment the compounds disclosed herein, e.g., Compound A, increases the sensitivity to chemotherapy. In one embodiment, the compounds disclosed herein, e.g., Compound A, increases the sensitivity to paclitaxel.

Compound 1 provided herein contains more than one chiral center, and can exist as a mixture of stereoisomers. This disclosure encompasses the use of stereomerically pure forms of such a compound, as well as the use of mixtures of those forms. For example, mixtures comprising equal or unequal amounts of the stereoisomers of Compound 1 provided herein may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, I N, 1972).

In certain embodiments, the compound for use in the compositions and methods provided herein is 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} (also referred to as docetaxel and Taxotere®), having the following structure:

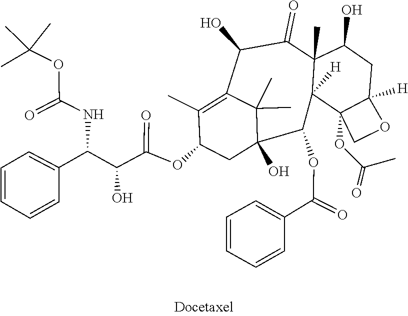

or a pharmaceutically acceptable salt, stereoisomer, tautomer, solid form, polymorph, hydrate, clathrate, or solvate thereof.

In one embodiment, the compound is 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}. In one embodiment, the compound is a pharmaceutically acceptable salt of docetaxel.

In one embodiment, the compound for use in the compositions and methods provided herein is pentyl [1-(3,4-dihydroxy-5-methyltetrahydrofuran-2-yl)-5-fluoro-2-oxo-1H-pyrimidin-4-yl]carbamate (also referred to as capecitabine and Xeloda®), having the following structure:

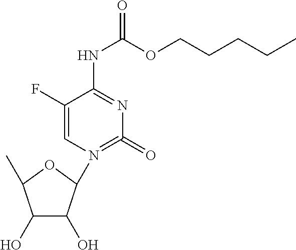

or a pharmaceutically acceptable salt, stereoisomer, tautomer, solid form, polymorph, hydrate, clathrate, or solvate thereof.

In one embodiment, the compound is pentyl [1-(3,4-dihydroxy-5-methyltetrahydrofuran-2-yl)-5-fluoro-2-oxo-1H-pyrimidin-4-yl]carbamate. In one embodiment, the compound is a pharmaceutically acceptable salt of capecitabine.

In certain embodiments, the compound for use in the compositions and methods provided herein is a monoclonal antibody that blocks PD-1. In particular, the anti-PD-1 antibody is nivolumab (also referred to as Opdivo®).

In one embodiment, the compound for use in the compositions and methods provided herein is (1S,2S,3R,4S,7R,9S,10S,12R,15S)-4,12-diacetoxy-15-{[(2R,3S)-3-(benzoylamino)-2-hydroxy-3-phenylpropanoyl]oxy}-1,9-dihydroxy-10,14,17,17-tetramethyl-11-oxo-6-oxatetracyclo[11.3.1.0˜3,10˜0.0˜4,7˜]heptadec-13-en-2-yl rel-benzoate also referred as to paclitaxel or Taxol®.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### Methods of Treatment and Compounds for Use in Such Methods

### Monotherapy

Compound A has utility as pharmaceutical to treat, manage or improve cancer in animals or humans. Further, Compound A is active against protein kinases, particularly JNK1 and/or JNK2. Accordingly, provided herein are uses of Compound A, including the treatment or management of those diseases set forth below.

Provided herein are methods of treating and/or managing cancer, which comprise administering to a patient having said cancer a therapeutically or prophylactically effective amount of Compound A. In one embodiment the cancer is a solid tumor. In one embodiment, the cancer is an advanced solid tumor. In one embodiment, the solid tumor is metastatic, refractory, recurrent, and/or unresectable. In one such embodiment, the solid tumor is immunotherapy (IO)-refractory.

In one embodiment, the solid tumor is lung cancer, breast cancer, squamous cell carcinoma of head and neck, pancreatic adenocarcinoma, pancreatic ductal adenocarcinoma, renal cell carcinoma, colorectal carcinoma, or sarcoma. In one such embodiment, the solid tumor is non-small cell lung cancer, triple negative breast cancer, or microsatellite-stable colorectal carcinoma. In one embodiment, the solid tumor is metastatic triple negative breast cancer. In one embodiment, the solid tumor is immunotherapy (IO)-refractory non small cell lung cancer.

The methods provided herein encompass methods for screening or identifying cancer patients, e.g., patients having lung cancer, breast cancer, squamous cell carcinoma of head and neck, pancreatic adenocarcinoma, pancreatic ductal adenocarcinoma, renal cell carcinoma, colorectal carcinoma, or sarcoma, for treatment with Compound A. In particular, provided herein are methods for selecting patients having, or who are likely to have, a higher response rate to a therapy with Compound A.

In one such embodiment, the patient has measurable disease, e.g., a solid tumor, which can be identified by a method well known in the art. In one embodiment, the patient is refractory to, not a candidate for, or intolerant of existing therapy(ies) known to provide clinical benefit for the condition of the patient. In one such embodiment, the patient has metastatic, recurrent, and/or unresectable non small cell lung cancer, metastatic triple negative breast cancer, squamous cell carcinoma of head and neck, pancreatic adenocarcinoma, pancreatic ductal adenocarcinoma, renal cell carcinoma, microsatellite-stable colorectal carcinoma, or sarcoma. In one such embodiment, the patient has measurable disease. In one such embodiment, the patient has advanced non small lung cancer or metastatic triple negative breast cancer with measurable disease.

In one particular embodiment, the patient is confirmed having advanced non-small cell lung cancer. In one such embodiment, the advanced non-small cell lung cancer has squamous or non-squamous histology that is resistant or refractory to antiprogrammed cell death 1 (PD-1)-based or anti-programmed cell death ligand 1 (PD-L1)-based treatment. In one embodiment, the patient has experienced progressive disease while on treatment. In one embodiment the patient has experienced progressive disease up to three months after the last dose of anti-PD-1 or anti-PD-L1 therapy.

In one embodiment, the patient is confirmed having metastatic triple negative breast cancer. In one embodiment, the patient has progressed, is intolerant, refractory, or not a candidate for at least two lines of treatment known to provide clinical benefit in the advanced setting. In one embodiment, the breast cancer is estrogen receptor and progesterone receptor≤1% by immunohistochemistry (IHC) and human epidermal growth factor receptor 2 (HER2) negative.

Provided herein are methods for treating or managing a solid tumor, comprising: (i) identifying a patient having a solid tumor sensitive to treatment with Compound A; and (ii) administering to the patient a therapeutically effective amount of Compound A. Provided herein is Compound A for use in such a method for treating or managing a solid tumor.

Provided herein are methods of treating cancer, e.g., a solid tumor, which result in an improvement in overall survival of the patient. In some embodiments, the improvement in overall survival of the patient is observed in a patient population sensitive to treatment with Compound A.

In one embodiment, provided herein are methods of treating cancer, e.g., a solid tumor, which result in disease free survival of the patient. In some embodiments, disease free survival of the patient is observed in a patient population sensitive to treatment with Compound A.

In one embodiment, provided herein are methods of treating cancer, e.g., a solid tumor, which result in an improvement in the objective response rate of the patient. In some embodiments, improvement in the objective response rate of the patient is observed in a patient population sensitive to treatment with Compound A.

In one embodiment, the solid tumor is lung cancer, breast cancer, squamous cell carcinoma of head and neck, pancreatic adenocarcinoma, pancreatic ductal adenocarcinoma, renal cell carcinoma, colorectal carcinoma, or sarcoma. In one such embodiment, the solid tumor is non-small cell lung cancer, triple negative breast cancer, or microsatellite-stable colorectal carcinoma. In one embodiment, the solid tumor is metastatic triple negative breast cancer. In one embodiment, the solid tumor is immunotherapy (IO)-refractory non small cell lung cancer.

In some embodiments, Compound A, is administered in combination with a therapy conventionally used to treat or manage cancer. Examples of such conventional therapies include, but are not limited to, surgery, chemotherapy, radiation therapy, hormonal therapy, biological therapy and immunotherapy.

Provided herein are methods of treating patients who have been previously treated for cancer but are non-responsive to standard therapies, as well as those who have not previously been treated. Provided herein are methods of treating patients who have been previously treated for solid tumors but are non-responsive to standard therapies, as well as those who have not previously been treated. The invention also encompasses methods of treating patients regardless of patient's age, although some diseases or disorders are more common in certain age groups. The invention further encompasses methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In certain embodiments, Compound A can be administered one to four times a day in a dose of about 0.005 mg/kg of a subject's body weight to about 15 mg/kg of a subject's body weight in a subject, but the above dosage may be properly varied depending on the age, body weight and medical condition of the subject and the type of administration. In one embodiment, the dose is about 0.01 mg/kg of a subject's body weight to about 10 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of Compound A administered will depend on such factors as the solubility of the active component, the formulation used and the route of administration.

In another embodiment, a therapeutically or prophylactically effective amount of Compound A is from about 0.375 mg/day to about 800 mg/day, about 0.75 mg/day to about 600 mg/day, about 0.75 mg/day to about 500 mg/day, about 0.75 mg/day to about 400 mg/day, about 0.75 mg/day to about 375 mg/day, about 0.75 mg/day to about 200 mg/day, 3.75 mg/day to about 75 mg/day, about 7.5 mg/day to about 55 mg/day, about 18 mg/day to about 37 mg/day, about 100 mg/day to about 800 mg/day, about 100 mg/day to about 700 mg/day, about 100 mg/day to about 600 mg/day, about 100 mg/day to about 500 mg/day, about 100 mg/day to about 400 mg/day, about 100 mg/day to about 300 mg/day, about 200 mg/day to about 800 mg/day, about 200 mg/day to about 700 mg/day, about 200 mg/day to about 600 mg/day, about 200 mg/day to about 500 mg/day, about 200 mg/day to about 400 mg/day, about 200 mg/day to about 300 mg/day of Compound A. In another embodiment, a therapeutically or prophylactically effective amount of Compound A is about 900 mg/day.

In another embodiment, a therapeutically or prophylactically effective amount of Compound A is from about 1 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 200 mg/day to about 800 mg/day, about 200 mg/day to about 600 mg/day, about 200 mg/day to about 400 mg/day, about 400 mg/day to about 800 mg/day, about 400 mg/day to about 600 mg/day or about 600 mg/day to about 800 mg/day of Compound A. In a particular embodiment, a therapeutically or prophylactically effective amount of Compound A is 200 mg/day, 400 mg/day, 600 mg/day or 800 mg/day of Compound A. In a particular embodiment, a therapeutically or prophylactically effective amount of Compound A is about 200 mg/day, about 300 mg/day, about 400 mg/day, about 500 mg, about 600 mg/day, about 700 mg/day, about 800 mg/day, or about 900 mg/day of Compound A. In a particular embodiment, a therapeutically or prophylactically effective amount of Compound A is 900 mg/day.

In another embodiment, provided herein are unit dosage formulations that comprise between about 1 mg and 200 mg, about 35 mg and about 1400 mg, about 125 mg and about 1000 mg, about 250 mg and about 1000 mg, about 500 mg and about 1000 mg, or about 200 mg and about 800 mg of Compound A.

In a particular embodiment, provided herein are unit dosage formulations comprising about 100 mg, 200 mg, 400 mg, or 600 mg of Compound A. In a particular embodiment, provided herein are unit dosage formulations comprising about 800 mg, or 900 mg of Compound A.

In another embodiment, provided herein are unit dosage formulations that comprise 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 300 mg, 350 mg, 375 mg, 400 mg, 450 mg, 500 mg, 560 mg, 600 mg, 700 mg, 750 mg, 800 mg, 900 mg, 1000 mg or 1400 mg of Compound A.

Compound A can be administered once, twice, three, four or more times daily. In a particular embodiment, doses of 600 mg or less are administered as a once daily dose and doses of more than 600 mg are administered twice daily in an amount equal to one half of the total daily dose. In one particular embodiment, Compound A is administered at a dose of 200 mg per day. In one particular embodiment, Compound A is administered at a dose of 300 mg per day. In one particular embodiment, Compound A is administered at a dose of 400 mg per day. In one particular embodiment, Compound A is administered at a dose of 500 mg per day. In one particular embodiment, Compound A is administered at a dose of 600 mg per day. In one particular embodiment, Compound A is administered at a dose of 700 mg per day. In one particular embodiment, Compound A is administered at a dose of 800 mg per day. In one particular embodiment, Compound A is administered at a dose of 900 mg per day.

Compound A can be administered orally for reasons of convenience. In one embodiment, when administered orally, Compound A is administered with a meal and water. In another embodiment, Compound A is dispersed in water or juice (e.g., apple juice or orange juice) and administered orally as a suspension.

Compound A can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

Pharmaceutical compositions and dosage forms of Compound A are also encompassed herein and further described below.

### Combination Therapies

Provided herein are methods of treating and/or managing cancer, which comprise administering to a patient having said cancer a therapeutically or prophylactically effective amount of Compound A as a part of a combination therapy. Provided herein is Compound A for use in such methods of treating and/or managing cancer.

Provided herein are methods of treating and/or managing cancer, which comprise administering to a patient having said cancer a therapeutically or prophylactically effective amount of Compound A and nivolumab. Provided herein is Compound A for use in such methods.

Provided herein are methods of treating and/or managing cancer, which comprise administering to a patient having said cancer a therapeutically or prophylactically effective amount of Compound A, and chemotherapy. Provided herein is Compound A for use in such methods of treating and/or managing cancer.

In one embodiment the cancer is a solid tumor. In one embodiment, the cancer is an advanced solid tumor. In one embodiment, the solid tumor is metastatic, refractory, recurrent, and/or unresectable. In one such embodiment, the solid tumor is immunotherapy (IO)-refractory.

In one embodiment, the solid tumor is lung cancer, breast cancer, squamous cell carcinoma of head and neck, pancreatic adenocarcinoma, pancreatic ductal adenocarcinoma, renal cell carcinoma, colorectal carcinoma, colon cancer, or sarcoma. In one embodiment, the solid tumor is pancreatic cancer. In one such embodiment, the solid tumor is non-small cell lung cancer, triple negative breast cancer, or microsatellite-stable colorectal carcinoma. In one embodiment, the solid tumor is non-small cell lung cancer (squamous subtype). In one embodiment, the solid tumor is non-small cell lung cancer (adenocarcinoma subtype). In one embodiment, the solid tumor is metastatic triple negative breast cancer. In one embodiment, the solid tumor is immunotherapy (IO)-refractory non small cell lung cancer.

In one embodiment, the chemotherapy comprises docetaxel or capecitabine. In one embodiment, the chemotherapy comprises paclitaxel, 5-FU, SN38, oxaliplatin, carboplatin, or gemcitabine. In one embodiment, the chemotherapy comprises paclitaxel.

In one embodiment, provided herein are methods of treating and/or managing cancer, which comprise administering to a patient having said cancer a therapeutically or prophylactically effective amount of Compound A, and docetaxel or a pharmaceutically acceptable salt, stereoisomer, tautomer, solid form, polymorph, hydrate, clathrate, or solvate thereof. Provided herein is Compound A for use in such methods of treating and/or managing cancer.

In one embodiment, provided herein are methods of treating and/or managing cancer, which comprise administering to a patient having said cancer a therapeutically or prophylactically effective amount of Compound A, and capecitabine or a pharmaceutically acceptable salt, stereoisomer, tautomer, solid form, polymorph, hydrate, clathrate, or solvate thereof. Provided herein is Compound A for use in such methods of treating and/or managing cancer.

The methods provided herein encompass methods for screening or identifying cancer patients, e.g., patients having lung cancer, breast cancer, squamous cell carcinoma of head and neck, pancreatic adenocarcinoma, pancreatic ductal adenocarcinoma, renal cell carcinoma, colorectal carcinoma, colon cancer or sarcoma, for treatment with Compound A. In particular, provided herein are methods for selecting patients having, or who are likely to have, a higher response rate to a therapy with Compound A in combination with nivolumab. In particular, provided herein are methods for selecting patients having, or who are likely to have, a higher response rate to a therapy with Compound A in combination with nivolumab.

Some methods provided herein encompass methods for screening or identifying cancer patients, e.g., patients having lung cancer, breast cancer, squamous cell carcinoma of head and neck, pancreatic adenocarcinoma, pancreatic ductal adenocarcinoma, renal cell carcinoma, colorectal carcinoma, colon cancer or sarcoma, for treatment with Compound A. In particular, provided herein are methods for selecting patients having, or who are likely to have, a higher response rate to a therapy with Compound A in combination with chemotherapy. In particular, provided herein are methods for selecting patients having, or who are likely to have, a higher response rate to a therapy with Compound A in combination with chemotherapy.

In one embodiment, the chemotherapy comprises docetaxel or capecitabine. In one embodiment, the chemotherapy comprises paclitaxel, 5-FU, SN38, oxaliplatin, carboplatin, or gemcitabine. In one embodiment, the chemotherapy comprises paclitaxel.

In one such embodiment, the patient has measurable disease, e.g., a solid tumor, which can be identified by a method well known in the art. In one embodiment, the patient is refractory to, not a candidate for, or intolerant of existing therapy(ies) known to provide clinical benefit for the condition of the patient. In one such embodiment, the patient has metastatic, recurrent, and/or unresectable non small cell lung cancer, metastatic triple negative breast cancer, squamous cell carcinoma of head and neck, pancreatic adenocarcinoma, pancreatic ductal adenocarcinoma, renal cell carcinoma, microsatellite-stable colorectal carcinoma, colon cancer or sarcoma. In one embodiment, the patient has pancreatic cancer. In one such embodiment, the patient has measurable disease. In one such embodiment, the patient has advanced non small lung cancer or metastatic triple negative breast cancer with measurable disease.

In one particular embodiment, the patient is confirmed having advanced non-small cell lung cancer. In one such embodiment, the advanced non-small cell lung cancer has squamous or non-squamous histology that is resistant or refractory to antiprogrammed cell death 1 (PD-1)-based or anti-programmed cell death ligand 1 (PD-L1)-based treatment. In one embodiment, the patient has non-small cell lung cancer (adenocarcinoma subtype). In one embodiment, the patient has experienced progressive disease while on treatment. In one embodiment the patient has experienced progressive disease up to three months after the last dose of anti-PD-1 or anti-PD-L1 therapy.

In one embodiment, the patient is confirmed having metastatic triple negative breast cancer. In one embodiment, the patient has progressed, is intolerant, refractory, or not a candidate for at least two lines of treatment known to provide clinical benefit in the advanced setting. In one embodiment, the breast cancer is estrogen receptor and progesterone receptor≤1% by immunohistochemistry (IHC) and human epidermal growth factor receptor 2 (HER2) negative.

Provided herein are methods for treating or managing a solid tumor, comprising: (i) identifying a patient having a solid tumor sensitive to treatment with Compound A; and (ii) administering to the patient a therapeutically effective amount of (a) Compound and (b) nivolumab. Provided herein is Compound A for use in such a method for treating or managing a solid tumor.

Also provided herein is a method for treating or managing a solid tumor, comprising: (i) identifying a patient having a solid tumor sensitive to treatment with Compound A; and (ii) administering to the patient a therapeutically effective amount of (a) Compound and (b) chemotherapy. Provided herein is Compound A for use in such a method for treating or managing a solid tumor.

In one embodiment, the chemotherapy comprises docetaxel or capecitabine. In one embodiment, the chemotherapy comprises paclitaxel, 5-FU, SN38, oxaliplatin, carboplatin, or gemcitabine. In one embodiment, the chemotherapy comprises paclitaxel.

Also provided herein is a method for treating or managing a solid tumor, comprising: (i) identifying a patient having a solid tumor sensitive to treatment with Compound A; and (ii) administering to the patient a therapeutically effective amount of (a) Compound and (b) docetaxel or a pharmaceutically acceptable salt, stereoisomer, tautomer, solid form, polymorph, hydrate, clathrate, or solvate thereof. Provided herein is Compound A for use in such a method for treating or managing a solid tumor.

Also provided herein is a method for treating or managing a solid tumor, comprising: (i) identifying a patient having a solid tumor sensitive to treatment with Compound A; and (ii) administering to the patient a therapeutically effective amount of (a) Compound A and (b) capecitabine or a pharmaceutically acceptable salt, stereoisomer, tautomer, solid form, polymorph, hydrate, clathrate, or solvate thereof. Provided herein is Compound A for use in such a method for treating or managing a solid tumor.

Provided herein are methods of treating or managing cancer, e.g., a solid tumor, which result in an improvement in overall survival of the patient. In some embodiments, the improvement in overall survival of the patient is observed in a patient population sensitive to treatment with (i) Compound A and (ii) nivolumab.

Also provided herein are methods of treating cancer, e.g., a solid tumor, which result in an improvement in overall survival of the patient. In some embodiments, the improvement in overall survival of the patient is observed in a patient population sensitive to treatment with (i) Compound A and (iii) chemotherapy. In one embodiment, the chemotherapy comprises docetaxel and capecitabine. In one embodiment, the chemotherapy comprises paclitaxel, 5-FU, SN38, oxaliplatin, carboplatin, or gemcitabine. In one embodiment, the chemotherapy comprises paclitaxel.

In other embodiments, provided herein are methods of treating cancer, e.g., a solid tumor, which result in disease free survival of the patient. In some embodiments, disease free survival of the patient is observed in a patient population sensitive to treatment with (i) Compound A and (ii) nivolumab.

In other embodiments, provided herein are methods of treating cancer, e.g., a solid tumor, which result in disease free survival of the patient. In some embodiments, disease free survival of the patient is observed in a patient population sensitive to treatment with (i) Compound A and (ii) chemotherapy. In one embodiment, the chemotherapy comprises docetaxel and capecitabine. In one embodiment, the chemotherapy comprises paclitaxel, 5-FU, SN38, oxaliplatin, carboplatin, or gemcitabine. In one embodiment, the chemotherapy comprises paclitaxel.

In other embodiments, provided herein are methods of treating cancer, e.g., a solid tumor, which result in an improvement in the objective response rate in the patient population. In some embodiments, the patient population sensitive to treatment with (i) Compound A and (ii) nivolumab.

In other embodiments, provided herein are methods of treating cancer, e.g., a solid tumor, which result in an improvement in the objective response rate in the patient population. In some embodiments, the patient population sensitive to treatment with (i) Compound A and (ii) chemotherapy. In one embodiment, the chemotherapy comprises docetaxel and capecitabine. In one embodiment, the chemotherapy comprises paclitaxel, 5-FU, SN38, oxaliplatin, carboplatin, or gemcitabine. In one embodiment, the chemotherapy comprises paclitaxel.

In one embodiment, the methods provided herein comprises docetaxel or a pharmaceutically acceptable salt, stereoisomer, tautomer, solid form, polymorph, hydrate, clathrate, or solvate thereof. In one embodiment, the methods provided herein comprises capecitabine or a pharmaceutically acceptable salt, stereoisomer, tautomer, solid form, polymorph, hydrate, clathrate, or solvate thereof. In one embodiment, the methods provided herein comprises paclitaxel or a pharmaceutically acceptable salt, stereoisomer, tautomer, solid form, polymorph, hydrate, clathrate, or solvate thereof.

In one embodiment, the solid tumor is lung cancer, breast cancer, squamous cell carcinoma of head and neck, pancreatic adenocarcinoma, pancreatic ductal adenocarcinoma, renal cell carcinoma, colorectal carcinoma, colon cancer or sarcoma. In one embodiment, the solid tumor is pancreatic cancer. In one such embodiment, the solid tumor is non-small cell lung cancer, triple negative breast cancer, or microsatellite-stable colorectal carcinoma. In one embodiment, the solid tumor is metastatic triple negative breast cancer. In one embodiment, the solid tumor is immunotherapy (IO)-refractory non small cell lung cancer.

In some embodiments, Compound A and nivolumab are administered in combination with a therapy conventionally used to treat or manage cancer. In some embodiments, Compound A and docetaxel or capecitabine are administered in combination with a therapy conventionally used to treat or manage cancer. Examples of such conventional therapies include, but are not limited to, surgery, chemotherapy, radiation therapy, hormonal therapy, biological therapy and immunotherapy.

In some embodiments, the methods for treating and/or managing a solid tumor provided herein may be used in patients that have not responded to standard treatment. In one embodiment, the solid tumor is metastatic, refractory, recurrent, and/or unresectable.

In other embodiments, the methods for treating a solid tumor provided herein may be used in treatment naive patients, i.e., patients that have not yet received treatment.

Provided herein are methods of treating patients who have been previously treated for cancer but are non-responsive to standard therapies, as well as those who have not previously been treated. Provided herein are methods of treating patients who have been previously treated for a solid tumor but are non-responsive to standard therapies, as well as those who have not previously been treated. Provided herein are methods of treating patients who have been previously treated for a solid tumor but are not responsive to immunotherapy. The invention also encompasses methods of treating patients regardless of patient's age, although some diseases or disorders are more common in certain age groups. The invention further encompasses methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

Provided herein are methods of treating patients who have been previously treated for cancer using at least two prior lines of therapy. Also provided herein are methods of treating patients who have been previously treated for metastatic breast cancer at least two prior lines of therapy. Also provided herein are methods of treating patients who have not been previously treated with capecitabine for metastatic cancer.

In certain embodiments, Compound A can be administered one to four times a day in a dose of about 0.005 mg/kg of a subject's body weight to about 15 mg/kg of a subject's body weight in a subject, but the above dosage may be properly varied depending on the age, body weight and medical condition of the subject and the type of administration. In one embodiment, the dose is about 0.01 mg/kg of a subject's body weight to about 10 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of Compound A administered will depend on such factors as the solubility of the active component, the formulation used and the route of administration.

In another embodiment, a therapeutically or prophylactically effective amount of Compound A is from about 0.375 mg/day to about 800 mg/day, about 0.75 mg/day to about 600 mg/day, about 0.75 mg/day to about 500 mg/day, about 0.75 mg/day to about 400 mg/day, about 0.75 mg/day to about 375 mg/day, about 0.75 mg/day to about 200 mg/day, 3.75 mg/day to about 75 mg/day, about 7.5 mg/day to about 55 mg/day, about 18 mg/day to about 37 mg/day, about 100 mg/day to about 800 mg/day, about 100 mg/day to about 700 mg/day, about 100 mg/day to about 600 mg/day, about 100 mg/day to about 500 mg/day, about 100 mg/day to about 400 mg/day, about 100 mg/day to about 300 mg/day, about 200 mg/day to about 800 mg/day, about 200 mg/day to about 700 mg/day, about 200 mg/day to about 600 mg/day, about 200 mg/day to about 500 mg/day, about 200 mg/day to about 400 mg/day, about 200 mg/day to about 300 mg/day of Compound A. In another embodiment, a therapeutically or prophylactically effective amount of Compound A is about 900 mg/day.

In another embodiment, a therapeutically or prophylactically effective amount of Compound A is from about 1 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 200 mg/day to about 800 mg/day, about 200 mg/day to about 600 mg/day, about 200 mg/day to about 400 mg/day, about 400 mg/day to about 800 mg/day, about 400 mg/day to about 600 mg/day, or about 600 mg/day to about 800 mg/day of Compound A. In a particular embodiment, a therapeutically or prophylactically effective amount of Compound A is 200 mg/day, 400 mg/day, 600 mg/day or 800 mg/day of Compound A. In a particular embodiment, a therapeutically or prophylactically effective amount of Compound A is about 200 mg/day, about 300 mg/day, about 400 mg/day, about 500 mg, about 600 mg/day, about 700 mg/day, about 800 mg/day, or about 900 mg/day of Compound A. In a particular embodiment, a therapeutically or prophylactically effective amount of Compound A is 900 mg/day.

In certain embodiments, a therapeutically or prophylactically effective amount of nivolumab is from about 0.5 to about 2,000 mg/kg per day, from about 1 to about 1,000 mg/kg per day, from about 1 to about 500 mg/kg per day, from about 1 to about 250 mg/kg per day, from about 5 to about 250 mg/kg per day, from about 7.5 to about 250 mg/kg per day, from about 10 to about 250 mg/kg per day, from about 16 to about 250 mg/kg per day, from about 16 to about 200 mg/kg per day, from about 1 to about 100 mg/kg per day, from about 1 to about 50 mg/kg per day, from about 0.5 to about 25 mg/kg per day, or from about 0.5 to about 10 mg/kg per day. In some embodiments, a therapeutically or prophylactically effective amount of nivolumab is from about 0.5 to about 2,000 mg/kg per day. In some embodiments, a therapeutically or prophylactically effective amount of nivolumab is from about 1 to about 1,000 mg/kg per day. In some embodiments, a therapeutically or prophylactically effective amount of nivolumab is from about 1 to about 500 mg/kg per day. In some embodiments, a therapeutically or prophylactically effective amount of nivolumab is from about 1 to about 250 mg/kg per day. In some embodiments, a therapeutically or prophylactically effective amount of nivolumab is from about 5 to about 250 mg/kg per day. In some embodiments, a therapeutically or prophylactically effective amount of nivolumab is from about 7.5 to about 250 mg/kg per day. In some embodiments, a therapeutically or prophylactically effective amount of nivolumab is from about 10 to about 250 mg/kg per day. In some embodiments, a therapeutically or prophylactically effective amount of nivolumab is from about 16 to about 250 mg/kg per day. In some embodiments, a therapeutically or prophylactically effective amount of nivolumab is from about 16 to about 200 mg/kg per day. In some embodiments, a therapeutically or prophylactically effective amount of nivolumab is from about 1 to about 100 mg/kg per day. In some embodiments, a therapeutically or prophylactically effective amount of nivolumab is from about 1 to about 50 mg/kg per day. In some embodiments, a therapeutically or prophylactically effective amount of nivolumab is from about 0.5 to about 25 mg/kg per day. In some embodiments, a therapeutically or prophylactically effective amount of nivolumab is from about 0.5 to about 10 mg/kg per day.

In certain embodiments, the therapeutically or prophylactically effective amount of nivolumab is about 0.5, about 1, about 2, about 3, about 5, about 10, about 15, about 20, about 25, about 30, about 40, about 45, about 50, about 60, about 70, about 80, about 90, about 100, about 150, or about 200 mg/kg per day. In certain embodiments, the therapeutically or prophylactically effective amount is about 0.5, about 1, about 2, about 5, about 10, about 15, about 20, about 25, about 30, about 40, about 45, about 50, about 60, about 70, about 80, about 90, about 100, about 150, or about 200 mg/kg per day. In some embodiments, the therapeutically or prophylactically effective amount of nivolumab is about 0.5 mg/kg per day. In some embodiments, the therapeutically or prophylactically effective amount of nivolumab is about 1 mg/kg per day. In some embodiments, the therapeutically or prophylactically effective amount of nivolumab is about 2 mg/kg per day. In some embodiments, the therapeutically or prophylactically effective amount of nivolumab is about 3 mg/kg per day. In some embodiments, the therapeutically or prophylactically effective amount of nivolumab is about 5 mg/kg per day. In some embodiments, the therapeutically or prophylactically effective amount of nivolumab is about 10 mg/kg per day. In some embodiments, the therapeutically or prophylactically effective amount of nivolumab is about 15 mg/kg per day. In some embodiments, the therapeutically or prophylactically effective amount of nivolumab is about 20 mg/kg per day. In some embodiments, the therapeutically or prophylactically effective amount of nivolumab is about 25 mg/kg per day. In some embodiments, the therapeutically or prophylactically effective amount of nivolumab is about 30 mg/kg per day. In some embodiments, the therapeutically or prophylactically effective amount of nivolumab is about 40 mg/kg per day. In some embodiments, the therapeutically or prophylactically effective amount of nivolumab is about 45 mg/kg per day. In some embodiments, the therapeutically or prophylactically effective amount of nivolumab is about 50 mg/kg per day. In some embodiments, the therapeutically or prophylactically effective amount of nivolumab is about 60 mg/kg per day. In some embodiments, the therapeutically or prophylactically effective amount of nivolumab is about 70 mg/kg per day. In some embodiments, the therapeutically or prophylactically effective amount of nivolumab is about 80 mg/kg per day. In some embodiments, the therapeutically or prophylactically effective amount of nivolumab is about 90 mg/kg per day. In some embodiments, the therapeutically or prophylactically effective amount of nivolumab is about 100 mg/kg per day. In some embodiments, the therapeutically or prophylactically effective amount of nivolumab is about 150 mg/kg per day. In some embodiments, the therapeutically or prophylactically effective amount of nivolumab is about 200 mg/kg per day.

In one embodiment, the recommended daily dose range of nivolumab lie within the range of from about 0.5 mg/kg to about 100 mg/kg per day, preferably given as a single once-a-day dose, or in divided doses throughout a day. In some embodiments, the dosage ranges from about 1 mg/kg to about 100 mg/kg per day. In other embodiments, the dosage ranges from about 0.5 to about 20 mg/kg per day. Specific doses per day include 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 mg/kg per day.

In certain embodiments, a therapeutically or prophylactically effective amount of nivolumab is from about 0.1 to about 100 mg/kg per day, from about 0.1 to about 50 mg/kg per day, from about 0.1 to about 20 mg/kg per day, from about 0.1 to about 10 mg/kg per day. Specific doses per day include about 0.5, 0.3, 1, or about 3 mg/kg per day. In another embodiment, nivolumab is administered in a dose of 1 mg/kg or more, such as a dose of from 1 to 20 mg/kg, e.g. a dose of from 5 to 20 mg/kg, e.g. a dose of 8 mg/kg. In some embodiments, a therapeutically or prophylactically effective amount of nivolumab is from about 0.1 to about 100 mg/kg per day. In some embodiments, a therapeutically or prophylactically effective amount of nivolumab is from about 0.1 to about 50 mg/kg per day. In some embodiments, a therapeutically or prophylactically effective amount of nivolumab is from about 0.1 to about 20 mg/kg per day. In some embodiments, a therapeutically or prophylactically effective amount of nivolumab is from about 0.1 to about 10 mg/kg per day. Specific doses per day include about 0.5 mg/kg per day. In some embodiments, a specific doses per day includes about 0.3 mg/kg per day. In some embodiments, a specific doses per day includes about 1 mg/kg per day. In some embodiments, a specific doses per day includes about 3 mg/kg per day. In some embodiments, a specific doses per day includes about 4 mg/kg per day. In some embodiments, a specific doses per day includes about 4.5 mg/kg per day.

In one embodiment, nivolumab is administered by infusion in a dosage of from about 10 to about 500 mg, such as of from about 300 to about 400 mg. In one embodiment, nivolumab is administered by infusion in a dosage of from 200 to 400 mg once every two weeks. In one embodiment, nivolumab is administered by infusion in a dosage of from about 300 to about 400 mg once every three weeks. In one embodiment, nivolumab is administered by infusion in a dosage of from about 300 to about 500 mg once every four weeks. In one embodiment, nivolumab is administered by infusion in a dosage of about 240 mg once every two weeks. In one embodiment, nivolumab is administered by infusion in a dosage of about 360 mg once every three weeks. In one embodiment, nivolumab is administered by infusion in a dosage of about 480 mg once every four weeks. The administration may be performed by continuous infusion over a period of from 30 minutes to 24 hours, such as of from 1 to 12 hours. In one embodiment, nivolumab is administered over a period of about 30 minutes. In one embodiment, nivolumab is administered by slow continuous infusion over a long period, such as over 1 hour in order to reduce toxic side effects.

In one embodiment, nivolumab is administered once every week, once every two weeks, or once every four weeks. In one specific embodiment, nivolumab is administered once every three weeks. In one embodiment, nivolumab is administered by maintenance therapy, such as, e.g., once every three weeks for a period of 6 months or more. In one embodiment, nivolumab is administered once every three weeks for a period of up to 2 years. In one embodiment, nivolumab is administered once every three weeks for a period of 6 months up to 2 years. In one specific embodiment, nivolumab is administered once every three weeks in a dosage of about 360 mg for a period of about 6 months. In one specific embodiment, nivolumab is administered once every three weeks in a dosage of about 360 mg for a period of about 6 months up to two years.

As non-limiting examples, treatment according to the present invention may be provided as a daily dosage of nivolumab in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In one embodiment, the method of treating or managing cancer provided herein comprises administering to a patient having said cancer (i) Compound A in an amount from about 200 mg to about 800 mg per day, and (ii) nivolumab in an amount of about 300 mg to about 400 mg every three weeks. In one embodiment, the method of treating or managing cancer provided herein comprises administering to a patient having said cancer (i) Compound A in an amount of about 400 mg per day, and (ii) nivolumab in an amount of about 360 mg every three weeks. In one embodiment, the method of treating or managing cancer provided herein comprises administering to a patient having said cancer (i) Compound A in an amount of about 600 mg per day, and (ii) nivolumab in an amount of about 360 mg every three weeks. In one embodiment, the method of treating or managing cancer provided herein comprises administering to a patient having said cancer (i) Compound 1, or a pharmaceutically acceptable salt, stereoisomer, tautomer, solid form, polymorph, hydrate, clathrate, or solvate thereof, in an amount of from about 200 mg to about 900 mg per day, and (ii) nivolumab in an amount of about 300 mg to about 400 mg every three weeks. In one embodiment, the method of treating or managing cancer provided herein comprises administering to a patient having said cancer (i) Compound A in an amount of about 900 mg per day, and (ii) nivolumab in an amount of about 360 mg every three weeks.

In certain embodiments, a therapeutically or prophylactically effective amount of docetaxel is from about 50 mg/m² to about 100 mg/m² per day, from about 55 mg/m² to about 85 mg/m² per day, or from about 65 mg/m² to about 80 mg/m² per day, or from about 70 mg/m² to about 75 mg/m² per day.

In certain embodiments, the therapeutically or prophylactically effective amount of docetaxel is about 50, about 55, about 60, about 65, about 70, about 75, about 80, about 85, about 90, about 95, about 100 mg/m² per day. In one embodiment, the therapeutically or prophylactically effective amount of docetaxel is about 60, about 61, about 62, about 63, about 64, about 65, about 66, about 67, about 68, about 69, about 70, about 71, about 72, about 73, about 74, about 75, about 76, about 77, about 78, about 79, about 80 mg/m² per day. In one embodiment, the therapeutically or prophylactically effective amount of docetaxel is about 50 mg/m² per day. In one embodiment, the therapeutically or prophylactically effective amount of docetaxel is about 60 mg/m² per day. In one embodiment, the therapeutically or prophylactically effective amount of docetaxel is about 70 mg/m² per day. In one embodiment, the therapeutically or prophylactically effective amount of docetaxel is about 75 mg/m² per day.

In one embodiment, the recommended daily dose range of docetaxel, or a pharmaceutically acceptable salt, stereoisomer, tautomer, solid form, polymorph, hydrate, clathrate, or solvate thereof, for the conditions described herein lie within the range of from about 50 mg/m² to about 100 mg/m² per day, preferably given as a single once-a-day dose, or in divided doses throughout a day. In some embodiments, the dosage ranges from about 60 mg/m² to about 80 mg/m² per day. In other embodiments, the dosage ranges from about 60 mg/m² to about 75 mg/m² per day. Specific doses include 60, 61, 62, 63, 64, 65, 66, 67, 68, 69, 70, 71, 72, 73, 74, 75, 76, 77, 78, 79, 80 mg/m² per day.

In one embodiment, docetaxel is administered by infusion in a weekly dosage of from about 50 mg/m² to about 100 mg/m², such as from 55 mg/m² to 85 mg/m², or from about 65 mg/m^{z} to about 80 mg/m², or from about 70 mg/m^{z} to about 75 mg/m². The administration may be performed by continuous infusion over a period of from 1 to 24 hours, such as of from 1 to 12 hours. In one embodiment, docetaxel is administered by continuous infusion over a period of at least 1 hour. In one embodiment, docetaxel is administered by slow continuous infusion over a long period, such as 12 hours, in order to reduce toxic side effects.

In one embodiment, docetaxel is administered by infusion once every three weeks at a dosage of from about 50 mg/m² to about 100 mg/m², such as from 55 mg/m² to 85 mg/m², or from about 65 mg/m² to about 80 mg/m², or from about 70 mg/m² to about 75 mg/m². In one embodiment, docetaxel is administered by infusion once every three weeks at a dosage of about 60 mg/m². In one embodiment, docetaxel is administered by infusion once every three weeks at a dosage of about 70 mg/m². In one embodiment, docetaxel is administered by infusion once every three weeks at a dosage of about 75 mg/m². The administration may be performed by continuous infusion over a period of from 1 to 24 hours, such as of from 1 to 12 hours. In one embodiment, docetaxel is administered by continuous infusion over a period of at least 1 hour. In one embodiment, docetaxel is administered by slow continuous infusion over a long period, such as 12 hours, in order to reduce toxic side effects. In one embodiment, docetaxel is administered by maintenance therapy, such as, e.g., once every three weeks for a period of 6 months or more. In one embodiment, docetaxel is administered by maintenance therapy, such as, e.g., once every three weeks for a period of up to 2 years. In one embodiment, docetaxel is administered by maintenance therapy, such as, e.g., once every three weeks for a period of 6 months up to 2 years.

In one embodiment, the method of treating or managing cancer provided herein comprises administering to a patient having said cancer (i) Compound A in an amount from about 200 mg to about 800 mg per day, and (ii) docetaxel in an amount of from about 50 mg/m² to about 100 mg/m² every three weeks. In one embodiment, the method of treating or managing cancer provided herein comprises administering to a patient having said cancer (i) Compound A in an amount of about 400 mg per day, and (ii) docetaxel in an amount of about 75 mg/m² every three weeks. In one embodiment, the method of treating or managing cancer provided herein comprises administering to a patient having said cancer (i) Compound A in an amount of about 600 mg per day, and (ii) docetaxel in an amount of about 75 mg/m² every three weeks. In one embodiment, the method of treating or managing cancer provided herein comprises administering to a patient having said cancer (i) Compound 1, or a pharmaceutically acceptable salt, stereoisomer, tautomer, solid form, polymorph, hydrate, clathrate, or solvate thereof, in an amount of from about 200 to about 900 mg per day, (ii) and docetaxel in an amount of from about 50 to about 100 mg/m² every three weeks. In one embodiment, the method of treating or managing cancer provided herein comprises administering to a patient having said cancer (i) Compound A in an amount of about 900 mg per day, and (ii) docetaxel in an amount of about 75 mg/m² every three weeks.

In certain embodiments, a therapeutically or prophylactically effective amount of capecitabine is from about 600 mg/m² to about 4000 mg/m² per day, from about 700 mg/m² to about 3000 mg/m², from about 800 mg/m² to about 2000 mg/m², from about 900 mg/m² to about 1000 mg/m² per day.

In certain embodiments, the therapeutically or prophylactically effective amount of capecitabine is about 600, about 650, about 700, about 750, about 800, about 850, about 900, about 950, about 1000, about 1150, about 1250, about 1350, about 1450, about 1550, about 1650, about 1750, about 1850, about 1950, about 2000, about 2250, about 2500, about 3000, about 3250, about 3500, about 4000 mg/m² per day. In one embodiment, the therapeutically or prophylactically effective amount of capecitabine is about 600 mg/m² per day. In one embodiment, the therapeutically or prophylactically effective amount of capecitabine is about 1000 mg/m² per day. In one embodiment, the therapeutically or prophylactically effective amount of capecitabine is about 1250 mg/m² per day. In one embodiment, the therapeutically or prophylactically effective amount of capecitabine is about 1500 mg/m² per day. In one embodiment, the therapeutically or prophylactically effective amount of capecitabine is about 1800 mg/m² per day. In one embodiment, the therapeutically or prophylactically effective amount of capecitabine is about 1900 mg/m² per day. In one embodiment, the therapeutically or prophylactically effective amount of capecitabine is about 2000 mg/m² per day. In one embodiment, the therapeutically or prophylactically effective amount of capecitabine is about 2500 mg/m² per day. In one embodiment, the therapeutically or prophylactically effective amount of capecitabine is about 4000 mg/m² per day.

In one embodiment, the recommended daily dose range of capecitabine, or a pharmaceutically acceptable salt, stereoisomer, tautomer, solid form, polymorph, hydrate, clathrate, or solvate thereof, for the conditions described herein lie within the range of from about 300 mg/m² to about 2000 mg/m², preferably given as a twice-a-day dose, or in divided doses throughout a day. In some embodiments, the dosage ranges from about 300 mg/m² to about 1500 mg/m² twice a day. In other embodiments, the dosage ranges from about 350 mg/m² to about 1250 mg/m² twice a day. Specific doses include 300, 325, 350, 375, 380, 385, 390, 395, 400, 425, 450, 475, 500, 525, 550, 575, 600, 625, 650, 675, 700, 725, 750, 775, 800, 825, 850, 875, 900, 925, 950, 975, 1000, 1025, 1050, 1100, 1150, 1200, 1250, 1300, 1350, 1400, 1450, 1500, 1550, 1600, 1650, 1700, 1750, 1800, 1850, 1900, 1950, or 2000 mg/m² twice a day. In one embodiment, specific doses include 1000 and/or 1250 mg/m² twice a day.

In one embodiment, capecitabine is administered orally as a twice-a-day dose at a dosage of from about 300 mg/m² to about 2000 mg/m², from about 300 mg/m² to about 1500 mg/m², or from about 350 mg/m² to about 1250 mg/m². In one embodiment, capecitabine is administered orally twice a day at a dosage of about 300 mg/m². In one embodiment, capecitabine is administered orally twice a day at a dosage of about 500 mg/m². In one embodiment, capecitabine is administered orally twice a day at a dosage of about 620 mg/m². In one embodiment, capecitabine is administered orally twice a day at a dosage of about 625 mg/m². In one embodiment, capecitabine is administered orally twice a day at a dosage of about 750 mg/m². In one embodiment, capecitabine is administered orally twice a day at a dosage of about 930 mg/m². In one embodiment, capecitabine is administered orally twice a day at a dosage of about 950 mg/m². In one embodiment, capecitabine is administered orally twice a day at a dosage of about 1000 mg/m². In one embodiment, capecitabine is administered orally twice a day at a dosage of about 1250 mg/m². In one embodiment, capecitabine is administered twice a day for two to four weeks. In one embodiment, capecitabine is administered twice a day for two weeks. In one embodiment, capecitabine is administered twice a day for three weeks. In one embodiment, capecitabine is administered twice a day for four weeks.

In one embodiment, the method of treating or managing cancer provided herein comprises administering to a patient having said cancer (i) Compound A in an amount from about 200 mg to about 800 mg per day, and (ii) capecitabine in an amount of from about 300 mg/m² to about 2000 mg/m² twice daily. In one embodiment, the method of treating or managing cancer provided herein comprises administering to a patient having said cancer (i) Compound A in an amount of about 400 mg per day, and (ii) capecitabine in an amount of about 1250 mg/m² twice daily. In one embodiment, the method of treating or managing cancer provided herein comprises administering to a patient having said cancer (i) Compound A in an amount of about 600 mg per day, and (ii) capecitabine in an amount of about 1250 mg/m² twice daily.

In one embodiment, the method of treating or managing cancer provided herein comprises administering to a patient having said cancer (i) Compound A in an amount from about 200 mg to about 900 mg per day, and (ii) capecitabine in an amount of from about 300 mg/m² to about 2000 mg/m² twice daily. In one embodiment, the method of treating or managing cancer provided herein comprises administering to a patient having said cancer (i) Compound 1, or a pharmaceutically acceptable salt, stereoisomer, tautomer, solid form, polymorph, hydrate, clathrate, or solvate thereof, in an amount of about 200, about 300, about 400, about 500, about 600 mg, about 800 mg, or about 900 mg per day, and (ii) capecitabine in an amount of about 300, about 400, about 500, about 600, about 700, about 800, about 900, about 1000, about 1100, about 1200 or about 1250 mg/m² twice daily. In one embodiment, the method of treating or managing cancer provided herein comprises administering to a patient having said cancer (i) Compound A in an amount of about 800 mg per day, and (ii) capecitabine in an amount of about 1000 mg/m² twice daily. In one embodiment, the method of treating or managing cancer provided herein comprises administering to a patient having said cancer (i) Compound A in an amount of about 900 mg per day, and (ii) capecitabine in an amount of about 1000 mg/m² twice daily.

In certain embodiments, a therapeutically or prophylactically effective amount of paclitaxel is from about 50 mg/m² to about 200 mg/m² per day, from about 100 mg/m² to about 200 mg/m² per day, or from about 120 mg/m² to about 180 mg/m² per day, or from about 125 mg/m² to about 175 mg/m² per day.

In certain embodiments, the therapeutically or prophylactically effective amount of paclitaxel is about 50, about 55, about 60, about 65, about 70, about 75, about 80, about 85, about 90, about 95, about 100, about 110, about 120, about 130, about 140, about 150, about 160, about 170, about 180, about 190, about 200 mg/m² per day. In one embodiment, the therapeutically or prophylactically effective amount of paclitaxel is about 130, about 131, about 132, about 133, about 134, about 135, about 136, about 137, about 138, about 139, about 140 mg/m² per day. In one embodiment, the therapeutically or prophylactically effective amount of docetaxel is about 160, about 161, about 162, about 163, about 164, about 165, about 166, about 167, about 168, about 169, about 170, about 171, about 172, about 173, about 174, about 175, about 176, about 177, about 178, about 179, about 180 mg/m² per day. In one embodiment, the therapeutically or prophylactically effective amount of paclitaxel is about 135 mg/m² per day. In one embodiment, the therapeutically or prophylactically effective amount of paclitaxel is about 175 mg/m² per day.

In one embodiment, the recommended daily dose range of paclitaxel, or a pharmaceutically acceptable salt, stereoisomer, tautomer, solid form, polymorph, hydrate, clathrate, or solvate thereof, for the conditions described herein lie within the range of from about 120 mg/m² to about 180 mg/m² per day, preferably given as a single once-a-day dose, or in divided doses throughout a day. In some embodiments, the dosage ranges from about 130 mg/m² to about 180 mg/m² per day. In other embodiments, the dosage ranges from about 135 mg/m² to about 175 mg/m² per day. Specific doses include 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 167, 168, 169, 170, 171, 172, 173, 174, 175 mg/m² per day.

In one embodiment, paclitaxel is administered by infusion in a weekly dosage of from about 120 mg/m² to about 180 mg/m², such as from about 130 mg/m² to about 180 mg/m², or from about 135 mg/m² to about 175 mg/m². The administration may be performed by continuous infusion over a period of from 1 to 24 hours, such as of from 1 to 12 hours. In one embodiment, paclitaxel is administered by continuous infusion over a period of at least 1 hour, or at least 3 hours. In one embodiment, paclitaxel is administered by slow continuous infusion over a long period, such as 12 hours, in order to reduce toxic side effects.

In one embodiment, paclitaxel is administered by infusion once every three weeks at a dosage of from about 120 mg/m² to about 180 mg/m², such as from about 130 mg/m² to about 180 mg/m², or from about 135 mg/m² to about 175 mg/m². In one embodiment, paclitaxel is administered by infusion once every three weeks at a dosage of about 135 mg/m². In one embodiment, paclitaxel is administered by infusion once every three weeks at a dosage of about 175 mg/m². The administration may be performed by continuous infusion over a period of from 1 to 24 hours, such as of from 1 to 12 hours. In one embodiment, paclitaxel is administered by continuous infusion over a period of at least 1 hour, or at least 3 hours. In one embodiment, paclitaxel is administered by slow continuous infusion over a long period, such as 12 hours, in order to reduce toxic side effects. In one embodiment, paclitaxel is administered by maintenance therapy, such as, e.g., once every three weeks for a period of 6 months or more. In one embodiment, paclitaxel is administered by maintenance therapy, such as, e.g., once every three weeks for a period of up to 2 years. In one embodiment, paclitaxel is administered by maintenance therapy, such as, e.g., once every three weeks for a period of 6 months up to 2 years.

In one embodiment, the method of treating or managing cancer provided herein comprises administering to a patient having said cancer (i) Compound A in an amount from about 200 mg to about 800 mg per day, and (ii) paclitaxel in an amount of from about 120 mg/m² to about 180 mg/m² every three weeks. In one embodiment, the method of treating or managing cancer provided herein comprises administering to a patient having said cancer (i) Compound A in an amount of about 400 mg per day, and (ii) paclitaxel in an amount of about 135 mg/m² every three weeks. In one embodiment, the method of treating or managing cancer provided herein comprises administering to a patient having said cancer (i) Compound A in an amount of about 600 mg per day, and (ii) paclitaxel in an amount of about 175 mg/m² every three weeks.

In certain embodiments, the patient to be treated with one of the methods provided herein has not been treated with anticancer therapy prior to the administration of Compound A and nivolumab. In certain embodiments, the patient to be treated with one of the methods provided herein has been treated with anticancer therapy prior to the administration of Compound A and nivolumab. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A and docetaxel, or a pharmaceutically acceptable salt, stereoisomer, tautomer, solid form, polymorph, hydrate, clathrate, or solvate thereof. In certain embodiments, the patient to be treated with one of the methods provided herein has been treated with anticancer therapy prior to the administration of Compound A and docetaxel, or a pharmaceutically acceptable salt, stereoisomer, tautomer, solid form, polymorph, hydrate, clathrate, or solvate thereof. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A and capecitabine, or a pharmaceutically acceptable salt, stereoisomer, tautomer, solid form, polymorph, hydrate, clathrate, or solvate thereof. In certain embodiments, the patient to be treated with one of the methods provided herein has been treated with anticancer therapy prior to the administration of Compound A and capecitabine, or a pharmaceutically acceptable salt, stereoisomer, tautomer, solid form, polymorph, hydrate, clathrate, or solvate thereof. In certain embodiments, the patient to be treated with one of the methods provided herein has developed drug resistance to the anticancer therapy.

The methods provided herein encompass treating a patient regardless of patient's age, although some diseases or disorders are more common in certain age groups. Further provided herein is a method for treating a patient who has undergone surgery in an attempt to treat the disease or condition at issue, as well in one who has not. Because the subjects with cancer have heterogeneous clinical manifestations and varying clinical outcomes, the treatment given to a particular subject may vary, depending on his/her prognosis. The skilled clinician will be able to readily determine without undue experimentation, specific secondary agents, types of surgery, and types of non-drug based standard therapy that can be effectively used to treat an individual subject with cancer.

Compound A may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Compound A may be formulated alone or together, in suitable dosage unit, with pharmaceutically acceptable excipients, carriers, adjuvants and vehicles, appropriate for each route of administration.

In one embodiment, Compound A is administered orally. In another embodiment, Compound A is administered parenterally. In yet another embodiment, Compound A is administered intravenously.

Compound A can be delivered as a single dose such as, e.g., a single bolus injection, or oral tablets or pills; or over time, such as, e.g., continuous infusion over time or divided bolus doses over time. Compound A and nivolumab can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

Compound A can be delivered as a single dose such as, e.g., a single bolus injection, or oral tablets or pills; or over time, such as, e.g., continuous infusion over time or divided bolus doses over time. Compound A and docetaxel, or a pharmaceutically acceptable salt, stereoisomer, tautomer, solid form, polymorph, hydrate, clathrate, or solvate thereof, can be administered repeatedly if necessary, for example, until the patient experiences stable disease or regression, or until the patient experiences disease progression or unacceptable toxicity. Compound A and capecitabine, or a pharmaceutically acceptable salt, stereoisomer, tautomer, solid form, polymorph, hydrate, clathrate, or solvate thereof, can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

Compound A can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug).

As used herein, the term “daily” is intended to mean that a therapeutic compound, such as Compound A, is administered once or more than once each day, for example, for a period of time. The term “continuous” is intended to mean that a therapeutic compound, such as Compound A, is administered daily for an uninterrupted period of at least 10 days to 52 weeks. The term “intermittent” or “intermittently” as used herein is intended to mean stopping and starting at either regular or irregular intervals. For example, intermittent administration of Compound A is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as Compound A, is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks.

In one embodiment, Compound A is administered once a day. In another embodiment, Compound A is administered twice a day. In yet another embodiment, Compound A is administered three times a day. In still another embodiment, Compound A is administered four times a day.

In one embodiment, nivolumab is administered once a day. In another embodiment, nivolumab is administered twice a day. In yet another embodiment, nivolumab is administered three times a day. In still another embodiment, nivolumab is administered four times a day. In still another embodiment, nivolumab is administered once every three weeks.

In one embodiment, docetaxel, or a pharmaceutically acceptable salt, stereoisomer, tautomer, solid form, polymorph, hydrate, clathrate, or solvate thereof, is administered once a day. In another embodiment, docetaxel, or a pharmaceutically acceptable salt, stereoisomer, tautomer, solid form, polymorph, hydrate, clathrate, or solvate thereof, is administered twice a day. In yet another embodiment, docetaxel, or a pharmaceutically acceptable salt, stereoisomer, tautomer, solid form, polymorph, hydrate, clathrate, or solvate thereof, is administered three times a day. In still another embodiment, docetaxel, or a pharmaceutically acceptable salt, stereoisomer, tautomer, solid form, polymorph, hydrate, clathrate, or solvate thereof, is administered four times a day. In still another embodiment, docetaxel, or a pharmaceutically acceptable salt, stereoisomer, tautomer, solid form, polymorph, hydrate, clathrate, or solvate thereof, is administered once every three weeks.

In one embodiment, capecitabine, or a pharmaceutically acceptable salt, stereoisomer, tautomer, solid form, polymorph, hydrate, clathrate, or solvate thereof, is administered once a day. In another embodiment, capecitabine, or a pharmaceutically acceptable salt, stereoisomer, tautomer, solid form, polymorph, hydrate, clathrate, or solvate thereof, is administered twice a day. In yet another embodiment, capecitabine, or a pharmaceutically acceptable salt, stereoisomer, tautomer, solid form, polymorph, hydrate, clathrate, or solvate thereof, is administered three times a day. In still another embodiment, capecitabine, or a pharmaceutically acceptable salt, stereoisomer, tautomer, solid form, polymorph, hydrate, clathrate, or solvate thereof, is administered four times a day.

In certain embodiments, Compound A is administered once per day from one day to two years, from one day to six months, from one week to three months, from one week to four weeks, from one week to three weeks, or from one week to two weeks. In certain embodiments, Compound A is administered once per day for one week, two weeks, ten weeks, twenty weeks, forty weeks, fifty weeks, sixty weeks, seventy weeks, eighty weeks, ninety weeks, or one hundred and four weeks. In one embodiment, Compound A is administered once per day for one week. In another embodiment, Compound A is administered once per day for two weeks. In yet another embodiment, Compound A is administered once per day for three weeks. In still another embodiment, Compound A is administered once per day for four weeks. In still another embodiment, Compound A is administered once per day for ten weeks. In still another embodiment, Compound A is administered once per day for twenty weeks. In still another embodiment, Compound A is administered once per day for 6 months. In still another embodiment, Compound A is administered once per day for one year. In still another embodiment, Compound A is administered once per day for two years.

In certain embodiments, Compound A is administered once per day for 21 days in each 21 day cycle. In certain embodiments, Compound A is administered for at least one cycle. In certain embodiments, Compound A is administered for one cycle. In certain embodiments, Compound A is administered for two cycles. In certain embodiments, Compound A is administered for three cycles. In certain embodiments, Compound A is administered for four cycles. In certain embodiments, Compound A is administered for five cycles. In certain embodiments, Compound A is administered for seven or more cycles.

In certain embodiments, nivolumab is administered once per day on days 1, 8, and 15, in each 21 day cycle. In certain embodiments, nivolumab is administered for at least one cycle. In certain embodiments, nivolumab is administered for at least two cycles. In certain embodiments, nivolumab is administered once per day on day 1 in each 21 day cycle. In certain embodiments, nivolumab is administered for one cycle. In certain embodiments, nivolumab is administered for two cycles. In certain embodiments, nivolumab is administered for three cycles. In certain embodiments, nivolumab is administered for four cycles. In certain embodiments, nivolumab is administered for five cycles. In certain embodiments, nivolumab is administered for seven or more cycles.

In certain embodiments, docetaxel, or a pharmaceutically acceptable salt, stereoisomer, tautomer, solid form, polymorph, hydrate, clathrate, or solvate thereof, is administered once per day on days 1, 8, and 15, in each 21 day cycle. In certain embodiments, docetaxel, or a pharmaceutically acceptable salt, stereoisomer, tautomer, solid form, polymorph, hydrate, clathrate, or solvate thereof, is administered for one cycle. In certain embodiments, docetaxel, or a pharmaceutically acceptable salt, stereoisomer, tautomer, solid form, polymorph, hydrate, clathrate, or solvate thereof, is administered for two cycles. In certain embodiments, docetaxel, or a pharmaceutically acceptable salt, stereoisomer, tautomer, solid form, polymorph, hydrate, clathrate, or solvate thereof, is administered once per day on day 1 in each 21 day cycle. In certain embodiments, docetaxel, or a pharmaceutically acceptable salt, stereoisomer, tautomer, solid form, polymorph, hydrate, clathrate, or solvate thereof, is administered for one cycle. In certain embodiments, docetaxel, or a pharmaceutically acceptable salt, stereoisomer, tautomer, solid form, polymorph, hydrate, clathrate, or solvate thereof, is administered for two cycles. In certain embodiments, docetaxel, or a pharmaceutically acceptable salt, stereoisomer, tautomer, solid form, polymorph, hydrate, clathrate, or solvate thereof, is administered for three cycles. In certain embodiments, docetaxel, or a pharmaceutically acceptable salt, stereoisomer, tautomer, solid form, polymorph, hydrate, clathrate, or solvate thereof, is administered for four cycles. In certain embodiments, docetaxel, or a pharmaceutically acceptable salt, stereoisomer, tautomer, solid form, polymorph, hydrate, clathrate, or solvate thereof, is administered for five cycles. In certain embodiments, docetaxel, or a pharmaceutically acceptable salt, stereoisomer, tautomer, solid form, polymorph, hydrate, clathrate, or solvate thereof, is administered for seven or more cycles. In certain embodiments, docetaxel, or a pharmaceutically acceptable salt, stereoisomer, tautomer, solid form, polymorph, hydrate, clathrate, or solvate thereof, is administered for four to six cycles.

In certain embodiments, capecitabine, or a pharmaceutically acceptable salt, stereoisomer, tautomer, solid form, polymorph, hydrate, clathrate, or solvate thereof, is administered twice per day on days 1, 8, and 15, in each 21 day cycle. In certain embodiments, capecitabine, or a pharmaceutically acceptable salt, stereoisomer, tautomer, solid form, polymorph, hydrate, clathrate, or solvate thereof, is administered for one cycle. In certain embodiments, capecitabine, or a pharmaceutically acceptable salt, stereoisomer, tautomer, solid form, polymorph, hydrate, clathrate, or solvate thereof, is administered for two cycles. In certain embodiments, capecitabine, or a pharmaceutically acceptable salt, stereoisomer, tautomer, solid form, polymorph, hydrate, clathrate, or solvate thereof, is administered twice per day two weeks on and one week off in each cycle. In certain embodiments, capecitabine, or a pharmaceutically acceptable salt, stereoisomer, tautomer, solid form, polymorph, hydrate, clathrate, or solvate thereof, is administered twice per day on day 1 and 8 in each 21 day cycle. In certain embodiments, capecitabine, or a pharmaceutically acceptable salt, stereoisomer, tautomer, solid form, polymorph, hydrate, clathrate, or solvate thereof, is administered for one cycle. In certain embodiments, capecitabine, or a pharmaceutically acceptable salt, stereoisomer, tautomer, solid form, polymorph, hydrate, clathrate, or solvate thereof, is administered for two cycles. In certain embodiments, capecitabine, or a pharmaceutically acceptable salt, stereoisomer, tautomer, solid form, polymorph, hydrate, clathrate, or solvate thereof, is administered for three cycles. In certain embodiments, capecitabine, or a pharmaceutically acceptable salt, stereoisomer, tautomer, solid form, polymorph, hydrate, clathrate, or solvate thereof, is administered for four cycles. In certain embodiments, capecitabine, or a pharmaceutically acceptable salt, stereoisomer, tautomer, solid form, polymorph, hydrate, clathrate, or solvate thereof, is administered for five cycles. In certain embodiments, capecitabine, or a pharmaceutically acceptable salt, stereoisomer, tautomer, solid form, polymorph, hydrate, clathrate, or solvate thereof, is administered for seven or more cycles.

In one embodiment, a compound disclosed herein is administered at an initial dose. In one embodiment, a compound disclosed herein is administered at an escalated dose from the initial dose. In one embodiment, a compound disclosed herein is administered at a reduced dose from the initial dose. In one embodiment, Compound A is administered at an escalated dose from the initial dose. In one embodiment, Compound A is administered at an initial dose of 400 mg per day and escalated to 600 mg per day. In one embodiment, Compound A is administered at an initial dose of 600 mg per day and escalated to 800 mg per day. In one embodiment, Compound A is administered at an initial dose of 600 mg per day and escalated to 900 mg per day. In one embodiment, Compound A is administered at an initial dose of 800 mg per day and escalated to 900 mg per day.

In one embodiment, a compound disclosed herein is administered at an initial dose. In one embodiment, a compound disclosed herein is administered at an escalated dose from the initial dose. In one embodiment, a compound disclosed herein is administered at a reduced dose from the initial dose. In one embodiment, docetaxel is administered at an initial dose of 75 mg/m² per day. In one embodiment, docetaxel is administered at a reduced dose of 60 mg/m² per day from the initial dose. In one embodiment, docetaxel is administered at a reduced dose of 60 mg/m² per day from the initial dose of 75 mg/m².

In one embodiment, a compound disclosed herein is administered at an initial dose. In one embodiment, a compound disclosed herein is administered at an escalated dose from the initial dose. In one embodiment, a compound disclosed herein is administered at a reduced dose from the initial dose. In one embodiment, capecitabine is administered at an initial dose of 1250 mg/m² twice a day. In one embodiment, capecitabine is administered at an initial dose of 1000 mg/m² twice a day. In one embodiment, capecitabine is administered at an initial dose of 1250 mg/m² twice a day for two weeks. In one embodiment, capecitabine is administered at an initial dose of 1000 mg/m² twice a day for two weeks. In one embodiment, capecitabine is administered at an initial dose of 1250 mg/m² twice a day for two weeks on and one week off. In one embodiment, capecitabine is administered at an initial dose of 1000 mg/m² twice a day for two weeks on and one week off. In one embodiment, capecitabine is administered at an initial dose of 1250 mg/m² twice a day and lowered to a dose of 1000 mg/m² twice a day. In one embodiment, capecitabine is administered at a reduced dose of 75%. In one embodiment, capecitabine is administered at a reduced dose of 75% from the initial dose. In one embodiment, capecitabine is administered at a reduced dose of 50%. In one embodiment, capecitabine is administered at a reduced dose of 50% from the initial dose. In one embodiment, capecitabine is administered at a first reduced dose of 75% from the initial dose. In one embodiment, capecitabine is administered at a second reduced dose of 50% from the first dose reduction. In one embodiment, capecitabine is administered at a reduced dose of 950 mg/m² per day from the initial dose. In one embodiment, capecitabine is administered at a reduced dose of 650 mg/m² per day from the initial dose. In one embodiment, capecitabine is administered at a reduced dose of 750 mg/m² per day from the initial dose. In one embodiment, capecitabine is administered at a reduced dose of 500 mg/m² per day from the initial dose.

### Pharmaceutical Compositions and Dosage Forms

In one embodiment, provided herein are pharmaceutical compositions and dosage forms, which comprise Compound A and one or more excipients.

In one embodiment, provided herein are pharmaceutical compositions and dosage forms, which comprise (i) Compound A and (ii) nivolumab. In another embodiment, pharmaceutical compositions and dosage forms further comprise one or more excipients.

In one embodiment, provided herein are pharmaceutical compositions and dosage forms, which comprise (i) Compound A and (ii) docetaxel. In another embodiment, pharmaceutical compositions and dosage forms further comprise one or more excipients.

In one embodiment, provided herein are pharmaceutical compositions and dosage forms, which comprise (i) Compound A and (ii) capecitabine. In another embodiment, pharmaceutical compositions and dosage forms further comprise one or more excipients.

In one embodiment, provided herein are pharmaceutical compositions and dosage forms, which comprise (i) Compound A and (ii) paclitaxel. In another embodiment, pharmaceutical compositions and dosage forms further comprise one or more excipients.

In certain embodiments, pharmaceutical compositions and dosage forms provided herein also comprise one or more additional active agents in amounts effective for achieving a modulation of disease or disease symptoms, including those described herein. Examples of optional additional active agents are disclosed herein (see, e.g., definitions section).

In certain embodiments, the pharmaceutical compositions provided herein may be administered orally, parenterally, by inhalation spray, topically, rectally, nasally, buccally, vaginally or via an implanted reservoir, preferably by oral administration or administration by injection. Oral delivery formats include, but are not limited to, tablets, capsules, caplets, solutions, suspensions, and syrups, and may also comprise a plurality of granules, beads, powders or pellets that may or may not be encapsulated. In one embodiment, the pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In certain embodiments, dosage forms provided herein for Compound A are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal, or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

In one embodiment, Compound A is formulated as disclosed in U.S. Pat. Nos. 9,796,685; 10,131,639; 10,590,089; and International Pub. No. WO2016/100310, the entirety of each of which is incorporated herein by reference.

In one embodiment, nivolumab is formulated as described in the package insert for OPDIVO®. As described therein, OPDIVO® is injected intravenously. OPDIVO® is available as a solution, at 40 mg/4 ml (10 mg/ml), 100 mg/10 ml (10 mg/ml), 120 mg/12 ml (10 mg/ml), or 240 mg/24 ml (10 mg/ml), in a single-dose vial. Each OPDIVO® single-dose vial contains nivolumab, mannitol, pentetic acid, polysorbate 80, sodium chloride, sodium citrate dihydrate, and water for Injection. The single-dose vial may contain hydrochloric acid and/or sodium hydroxide to adjust pH to 6. The single-dose vial is diluted with either 0.9% Sodium Chloride injection or 5% Dextrose injection to prepare an infusion with a final concentration ranging from 1 mg/ml to 10 mg/ml.

In one embodiment, docetaxel is formulated as described in the package insert for TAXOTERE®. As described therein, TAXOTERE® is injected intravenously. TAXOTERE® is available as a solution, at 20 mg/ml, 80 mg/4 ml, 160 mg/8 ml, in a single-dose vial. Alternatively, TAXOTERE® is available as a solution, at 20 mg/2 ml, 80 mg/8 ml, 160 mg/16 ml, in a multi-dose vial. Each single-dose vial contains polysorbate 80/dehydrated alcohol. Single-dose vials at 20 mg/ml do not require dilution with a diluent.

In one embodiment, provided herein capecitabine is administered orally. In one embodiment, capecitabine is provided herein as a tablet. In one embodiment, capecitabine is formulated as described in the package insert for XELODA®. As described therein, XELODA® is available in 150 mg and 500 mg tablets each filmed coated. Each tablet contains anhydrous lactose, croscarmellose sodium, hydroxypropyl methylcellulose, microcrystalline cellulose, magnesium stearate and purified water. The film coating contains hydroxypropyl methylcellulose, talc, titanium dioxide, and synthetic yellow and red iron oxides.

In one embodiment, paclitaxel is formulated as described in the package insert for TAXOL®. As described therein, TAXOL® is injected intravenously. TAXOL® is available as 30 mg (5 mL), 100 mg (16.7 mL), and 300 mg (50 mL) multidose vials. Each mL of sterile nonpyrogenic solution contains 6 mg paclitaxel, 527 mg of purified Cremophor® EL* (polyoxyethylated castor oil) and 49.7% (v/v) dehydrated alcohol, USP.

Whether a particular excipient is suitable for incorporation into a pharmaceutical composition or dosage form provided herein depends on a variety of factors, including, but not limited to, the route of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, encompassed herein are pharmaceutical compositions and dosage forms that contain little, if any, lactose. As used herein, the term “lactose-free” means that the amount of lactose present, if any, is insufficient to substantially increase the degradation rate of an active ingredient.

Lactose-free compositions provided herein can comprise excipients that are listed, for example, in the U.S. Pharmacopeia (USP) 25-NF20 (2002). In certain embodiments, lactose-free compositions comprise active ingredients, a binder/filler, and a lubricant in pharmaceutically compatible and pharmaceutically acceptable amounts. In certain embodiments, lactose-free dosage forms comprise active ingredients, microcrystalline cellulose, pre-gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, in certain embodiments, provided herein are anhydrous compositions packaged using materials to prevent exposure to water such that they can be included in suitable formulary kits. Examples of suitable packaging include, but are not limited to, hermetically sealed foils, plastics, unit dose containers (e.g., vials), blister packs, and strip packs.

Encompassed herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

### Oral Dosage Forms

In certain embodiments, pharmaceutical compositions provided herein that are suitable for oral administration are formulated as discrete dosage forms, examples of which include, but are not limited to, tablets (e.g., chewable tablets), caplets, capsules, and liquids (e.g., flavored syrups). Such dosage forms contain predetermined amounts of active ingredients and may be prepared by some known methods of pharmacy. See generally, Remington's Pharmaceutical Sciences, 18th ed., Mack Publishing, Easton PA (1990).

In certain embodiments, the oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms may be prepared by some known methods of pharmacy. In certain embodiments, pharmaceutical compositions and dosage forms are prepared by uniformly and intimately admixing the active ingredients with liquid carriers, finely divided solid carriers, or both, and then shaping the product into the desired presentation if necessary.

In certain embodiments, a tablet is prepared by compression or molding. In certain embodiments, compressed tablets are prepared by compressing in a suitable machine the active ingredients in a free-flowing form, e.g., powder or granules, optionally mixed with an excipient. In certain embodiments, molded tablets are made by molding in a suitable machine a mixture of a powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, AVICEL-PH-101, AVICEL-PH-103 AVICEL RC-581, AVICEL-PH-105 (FMC Corporation, American Viscose Division, Avicel Sales, Marcus Hook, PA), and mixtures thereof. An specific binder is a mixture of microcrystalline cellulose and sodium carboxymethyl cellulose (e.g., AVICEL RC-581). Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein is present in from about 50 to about 99 weight percent of the pharmaceutical composition or dosage form.

Disintegrants are used in the compositions provided herein to provide tablets the ability to disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation. In certain embodiments, the pharmaceutical compositions provided herein comprise from about 0.5 to about 15 weight percent or from about 1 to about 5 weight percent of disintegrant.

Disintegrants that are suitable for use in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that are suitable for use in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, but are not limited to, a syloid silica gel (AEROSIL200, W.R. Grace Co., Baltimore, MD), a coagulated aerosol of synthetic silica (Degussa Co. of Plano, TX), CAB-O-SIL (a pyrogenic silicon dioxide, Cabot Co. of Boston, MA), and mixtures thereof. In certain embodiments, if used at all, lubricants are used in an amount of less than about 1 weight percent of the pharmaceutical compositions or dosage forms into which they are incorporated.

In certain embodiments, provided herein is a solid oral dosage form, comprising Compound A and one or more pharmaceutically acceptable excipients or carriers, each independently selected from mannitol, cellulose, hydroxypropyl methycellulose (HPMC), carboxymethyl cellulose and magnesium stearate. In one embodiment, the solid oral dosage form is formulated in a single unit dosage form. In one specific embodiment, the single unit dosage forms are 30 mg, 100 mg, 200 mg, 400 mg, or 600 mg tablets. In one specific embodiment, the single unit dosage forms are 200 mg or 400 mg tablets.

In certain embodiments, provided herein is a solid oral dosage form, comprising Compound A and mannitol, cellulose, hydroxypropyl methycellulose (HPMC), carboxymethyl cellulose and magnesium stearate. In one embodiment, the solid oral dosage form is formulated in a single unit dosage form. In one specific embodiment, the single unit dosage forms are 200 mg or 400 mg tablets. In one embodiment, the solid oral dosage form is formulated in a single unit dosage form. In one specific embodiment, the single unit dosage forms are 30 mg, 100 mg, 200 mg, or 400 mg tablets. In one specific embodiment, the single unit dosage forms are 200 mg or 400 mg tablets.

### Delayed Release Dosage Forms

In certain embodiments, the active ingredients provided herein are administered by controlled release means or by delivery devices. Examples include, but are not limited to, those described in U.S. Pat. Nos. 3,845,770; 3,916,899; 3,536,809; 3,598,123; 4,008,719, 5,674,533, 5,059,595, 5,591,767, 5,120,548, 5,073,543, 5,639,476, 5,354,556, and 5,733,566, each of which is incorporated herein by reference in its entirety. In certain embodiments, such dosage forms are be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Encompassed herein are single unit dosage forms suitable for oral administration, including, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Some suitable vehicles that can be used to provide parenteral dosage forms provided herein include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton PA (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein depend on the particular tissue to which a given pharmaceutical composition or dosage form will be applied. With that fact in mind, in certain embodiments, the excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Additional examples of such ingredients can be found, e.g., in Remington's Pharmaceutical Sciences, 16^{th} and 18^{th} eds., Mack Publishing, Easton PA (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Kits

In certain embodiments, active ingredients provided herein are not administered to a patient at the same time or by the same route of administration. Therefore, encompassed herein are kits which, when used by the medical practitioner, can simplify the administration of appropriate amounts of active ingredients to a patient.

In certain embodiments, a kit provided herein comprises a dosage form of a Compound A. In certain embodiments, the kits provided herein further comprise nivolumab. In certain embodiments, the kit provided herein further comprises additional active ingredient(s) include, but are not limited to, those disclosed herein.

In certain embodiments, a kit provided herein comprises a dosage form of a Compound A. In certain embodiments, the kits provided herein further comprise docetaxel or capecitabine. In certain embodiments, the kit provided herein further comprises additional active ingredient(s) include, but are not limited to, those disclosed herein.

In certain embodiments, the kit provided herein further comprises a device that is used to administer the active ingredients. Examples of such devices include, but are not limited to, syringes, drip bags, patches, and inhalers.

In certain embodiments, the kit provided herein further comprises cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Embodiments of the present disclosure include:
Embodiment 1. A method of treating or managing cancer, comprising administering to a patient having said cancer a therapeutically effective amount of Compound 1

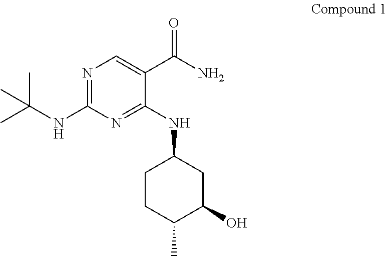 [SMILES: C[C@@H]1CC[C@@H](Nc2nc(NC(C)(C)C)ncc2C(N)=O)C[C@H]1O]

or a pharmaceutically acceptable salt, stereoisomer, tautomer, solid form, polymorph, hydrate, clathrate, or solvate thereof.
Embodiment 2. A method of treating or managing cancer, comprising administering to a patient having said cancer a therapeutically effective amount of (i) Compound

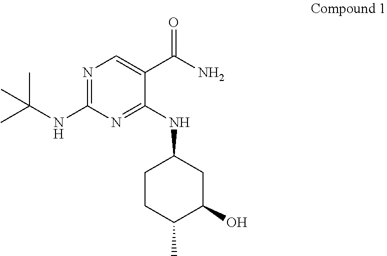 [SMILES: C[C@@H]1CC[C@@H](Nc2nc(NC(C)(C)C)ncc2C(N)=O)C[C@H]1O]

or a pharmaceutically acceptable salt, stereoisomer, tautomer, solid form, polymorph, hydrate, clathrate, or solvate thereof and (ii) nivolumab.
Embodiment 3. A method of treating or managing cancer, comprising administering to a patient having said cancer a therapeutically effective amount of (i) Compound 1

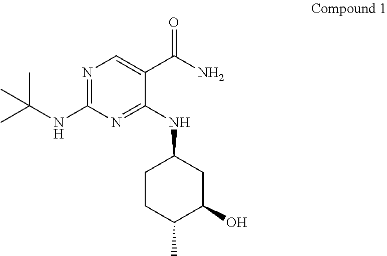 [SMILES: C[C@@H]1CC[C@@H](Nc2nc(NC(C)(C)C)ncc2C(N)=O)C[C@H]1O]

or a pharmaceutically acceptable salt, stereoisomer, tautomer, solid form, polymorph, hydrate, clathrate, or solvate thereof and (ii) chemotherapy.
Embodiment 4. The method of any one of embodiments 1 to 3, wherein the cancer is a solid tumor.
Embodiment 5. The method of embodiment 4, wherein the solid tumor is lung cancer, breast cancer, squamous cell carcinoma of head and neck, pancreatic adenocarcinoma, pancreatic ductal adenocarcinoma, renal cell carcinoma, colorectal carcinoma, or sarcoma.
Embodiment 6. The method of embodiment 4, wherein the solid tumor is non-small cell lung cancer, triple negative breast cancer, or microsatellite-stable colorectal carcinoma.
Embodiment 7. The method of embodiment 6, wherein the triple negative breast cancer is metastatic triple negative breast cancer.
Embodiment 8. The method of embodiment 4, wherein the solid tumor is metastatic, refractory, recurrent, and/or unresectable.
Embodiment 9. The method of embodiment 4 or 5, wherein the solid tumor is immunotherapy-refractory.
Embodiment 10. The method of any one of embodiments 3 to 9, wherein the chemotherapy is docetaxel or capecitabine.
Embodiment 11. A method for treating or managing a solid tumor, comprising:
(i) identifying a patient having a solid tumor sensitive to treatment with Compound 1, or a pharmaceutically acceptable salt, stereoisomer, tautomer, solid form, polymorph, hydrate, clathrate, or solvate thereof; and
(ii) administering to the patient a therapeutically effective amount of Compound 1, or a pharmaceutically acceptable salt, stereoisomer, tautomer, solid form, polymorph, hydrate, clathrate, or solvate thereof.
Embodiment 12. A method for treating or managing a solid tumor, comprising:
(i) identifying a patient having a solid tumor sensitive to treatment with (a) Compound 1, or a pharmaceutically acceptable salt, stereoisomer, tautomer, solid form, polymorph, hydrate, clathrate, or solvate thereof and (b) nivolumab; and
(ii) administering to the patient a therapeutically effective amount of (a) Compound 1, or a pharmaceutically acceptable salt, stereoisomer, tautomer, solid form, polymorph, hydrate, clathrate, or solvate thereof and (b) nivolumab.
Embodiment 13. A method for treating or managing a solid tumor, comprising:
(i) identifying a patient having a solid tumor sensitive to treatment with (a) Compound 1, or a pharmaceutically acceptable salt, stereoisomer, tautomer, solid form, polymorph, hydrate, clathrate, or solvate thereof and (b) a chemotherapy; and
(ii) administering to the patient a therapeutically effective amount of (a) Compound 1, or or a pharmaceutically acceptable salt, stereoisomer, tautomer, solid form, polymorph, hydrate, clathrate, or solvate thereof and (b) a chemotherapy.
Embodiment 14. The method of any one of embodiments 11 to 13, wherein the solid tumor is metastatic, refractory, recurrent, and/or unresectable.
Embodiment 15. The method of any one of embodiments 11 to 13, wherein the solid tumor is lung cancer, breast cancer, squamous cell carcinoma of head and neck, pancreatic adenocarcinoma, pancreatic ductal adenocarcinoma, renal cell carcinoma, colorectal carcinoma, or sarcoma.
Embodiment 16. The method of embodiment 15, wherein the solid tumor is non-small cell lung cancer, triple negative breast cancer, or microsatellite-stable colorectal carcinoma.
Embodiment 17. The method of any one of embodiments 13 to 16, wherein the chemotherapy is docetaxel or capecitabine.
Embodiment 18. The method of any one of embodiments 12 to 17, wherein the solid tumor is immunotherapy refractory non-small lung cancer.
Embodiment 19. The method of any one of embodiments 12 to 17, wherein the solid tumor is metastatic triple negative breast cancer.
Embodiment 20. The method of any one of embodiments 1 to 19, wherein Compound 1, or a pharmaceutically acceptable salt, stereoisomer, tautomer, solid form, polymorph, hydrate, clathrate, or solvate thereof, is administered in an amount of from about 200 mg to about 800 mg per day.
Embodiment 21. The method of any one of embodiments 1 to 19, wherein (i) Compound 1, or a pharmaceutically acceptable salt, stereoisomer, tautomer, solid form, polymorph, hydrate, clathrate, or solvate thereof, is administered in an amount of from about 200 mg to about 800 mg per day, and (ii) nivolumab is administered in an amount of about 300 mg to about 400 mg every three weeks.
Embodiment 22. The method of any one of embodiments 1 to 19, wherein (i) Compound 1, or a pharmaceutically acceptable salt, stereoisomer, tautomer, solid form, polymorph, hydrate, clathrate, or solvate thereof, is administered in an amount of from about 200 to about 800 mg per day, (ii) and docetaxel is administered in an amount of from about 50 to about 100 mg/m² every three weeks.
Embodiment 23. The method of any one of embodiments 1 to 19, wherein (i) Compound 1, or a pharmaceutically acceptable salt, stereoisomer, tautomer, solid form, polymorph, hydrate, clathrate, or solvate thereof, is administered in an amount of from about 200 to about 800 mg per day, (ii) and capecitabine is administered in an amount of from about 300 to about 2000 mg/m² twice daily.
Embodiment 24. The method of embodiment 21, wherein (i) Compound 1, or a pharmaceutically acceptable salt, stereoisomer, tautomer, solid form, polymorph, hydrate, clathrate, or solvate thereof, is administered in an amount of about 200, about 300, about 400, about 500, or about 600 mg per day, and (ii) nivolumab is administered in an amount of about 360 mg every three weeks.
Embodiment 25. The method of embodiment 21, wherein (i) Compound 1, or a pharmaceutically acceptable salt, stereoisomer, tautomer, solid form, polymorph, hydrate, clathrate, or solvate thereof, is administered orally and (ii) nivolumab is administered intravenously.
Embodiment 26. The method of embodiment 21, wherein (i) Compound 1, or a pharmaceutically acceptable salt, stereoisomer, tautomer, solid form, polymorph, hydrate, clathrate, or solvate thereof, is administered by a capsule or a tablet and (ii) nivolumab is administered by injection or infusion.
Embodiment 27. The method of embodiment 26, wherein (i) Compound 1, or a pharmaceutically acceptable salt, stereoisomer, tautomer, solid form, polymorph, hydrate, clathrate, or solvate thereof, is administered in 200 mg or 400 mg of a tablet and (ii) nivolumab is administered as intravenous infusion of 40 mg/4 ml, 100 mg/10 ml, 240 mg/24 ml, single dose vial.
Embodiment 28. The method of embodiment 22, wherein (i) Compound 1, or a pharmaceutically acceptable salt, stereoisomer, tautomer, solid form, polymorph, hydrate, clathrate, or solvate thereof, is administered in an amount of about 200, about 300, about 400, about 500, or about 600 mg per day, and (ii) docetaxel is administered in an amount of about 60, about 70, or about 75 mg/m² every three weeks.
Embodiment 29. The method of embodiment 22, wherein (i) Compound 1, or a pharmaceutically acceptable salt, stereoisomer, tautomer, solid form, polymorph, hydrate, clathrate, or solvate thereof, is administered orally, and (ii) docetaxel is administered intravenously.
Embodiment 30. The method of embodiment 22, wherein (i) Compound 1, or a pharmaceutically acceptable salt, stereoisomer, tautomer, solid form, polymorph, hydrate, clathrate, or solvate thereof, is administered by a capsule or tablet, and (ii) docetaxel is administered by injection or infusion.
Embodiment 31. The method of embodiment 30, wherein (i) Compound 1, or a pharmaceutically acceptable salt, stereoisomer, tautomer, solid form, polymorph, hydrate, clathrate, or solvate thereof, is administered in 200 mg or 400 mg of a tablet, and (ii) docetaxel is administered as intravenous infusion of 20 mg/2 ml, 80 mg/8 mL, or 160 mg/16 mL multi dose vial.
Embodiment 32. The method of embodiment 23, wherein (i) Compound 1, or a pharmaceutically acceptable salt, stereoisomer, tautomer, solid form, polymorph, hydrate, clathrate, or solvate thereof, is administered in an amount of about 200, about 300, about 400, about 500, or about 600 mg per day, and (ii) capecitabine is administered in an amount of about 300, about 400, about 500, about 600, about 700, about 800, about 900, about 1000, about 1100, about 1200 or about 1250 mg/m² twice daily.
Embodiment 33. The method of embodiment 23, wherein (i) Compound 1, or a pharmaceutically acceptable salt, stereoisomer, tautomer, solid form, polymorph, hydrate, clathrate, or solvate thereof, is administered orally, and (ii) capecitabine is administered orally. Embodiment 34. The method of embodiment 23, wherein (i) Compound 1, or a pharmaceutically acceptable salt, stereoisomer, tautomer, solid form, polymorph, hydrate, clathrate, or solvate thereof, is administered by capsule or tablet, and (ii) capecitabine is administered in a capsule or tablet.
Embodiment 35. The method of embodiment 23, wherein (i) Compound 1, or a pharmaceutically acceptable salt, stereoisomer, tautomer, solid form, polymorph, hydrate, clathrate, or solvate thereof, is administered in 200 mg or 400 mg of a tablet, and (ii) capecitabine is administered in 150 mg or 500 mg of a tablet.
Embodiment 36. The method of any one of embodiments 1-35, wherein the solid tumor is metastatic, refractory, recurrent, and/or unresectable.
Embodiment 37. The method of embodiment 22, wherein the docetaxel is administered in an amount of about 75 mg/m² every three weeks.
Embodiment 38. The method of embodiment 37, wherein the docetaxel is administered at least 4 to 6 cycles.
Embodiment 39. The method of embodiment 23, wherein the capecitabine is administered in an amount of about 1250 mg/m² twice daily.
Embodiment 40. The method of embodiment 39, wherein the capecitabine is administered 2 weeks on and 1 week off.
Embodiment 41. The method of any one of embodiments 1-40, wherein the compounds are administered in a 21 day cycle.
Embodiment 42. The method of embodiment 21 or 22, wherein the solid tumor is immunotherapy refractory non small lung cancer.
Embodiment 43. The method of embodiment 23, wherein the solid tumor is metastatic triple negative breast cancer.

### EXAMPLES

Certain embodiments of the invention are illustrated by the following non-limiting examples.

### Example 1: A Phase 1 Study of Compound a Alone and in Combination with Chemotherapy or Nivolumab in Advanced Solid Tumors

This study aims to establish preliminary safety and efficacy data of Compound A in participants with advanced solid tumors where the INK pathway is thought to play a pivotal role in disease biology. In addition, the pharmacodynamic (PD) profile of Compound A in human tumors will be evaluated.

Data suggest that Compound A can achieve the desired PD profile at a clinically tolerable dose, and ultimately enhance the response to chemotherapy or nivolumab in patients with advanced solid tumors.

### I. Overall Design

This is a Phase 1, open-label, multicenter study of Compound A, administered alone and in combination with chemotherapy or nivolumab, in participants with selected advanced solid tumors: non-small cell lung cancer (NSCLC), metastatic triple negative breast cancer (mTNBC), pancreatic adenocarcinoma (PAAD), squamous cell carcinoma of head and neck (SCCHN), renal cell carcinoma (RCC), microsatellite-stable colorectal carcinoma (MSS CRC), and sarcoma. The study comprises 2 parts:

Part 1 includes dose escalation of Compound A alone in the following advanced solid tumors indications: immunotherapy (10)-refractory NSCLC, mTNBC, PAAD, SCCHN, RCC, MSS CRC, and sarcoma, and will inform the recommended dose(s) for Part 2 of the study.

Part 2 includes two parts:

Part 2A: a dose escalation for Compound A in combination with docetaxel or nivolumab followed by expansion cohorts of Compound A alone or in combination with docetaxel or nivolumab in participants with JO-refractory NSCLC.

Part 2B: a dose escalation followed by an expansion cohort for Compound A in combination with capecitabine in participants with mTNBC. The dose to be evaluated in each expansion cohort will be based on the totality of safety, pharmacokinetic (PK), and PD data from all participants in Part 1 and the dose escalation cohorts in Part 2.

Efficacy assessments for the anti-tumor activity of Compound A alone, in combination with docetaxel or capecitabine, or in combination with nivolumab will be based on tumor measurements, using Response Evaluation Criteria in Solid Tumors (RECIST) v1.1, with computed tomography (CT) and/or magnetic resonance imaging (MRI), as appropriate. Assessments will be performed at baseline and every 9 weeks (±7 days).

Two tumor biopsies are required in all participants in Part 2 of the study: one prior to starting treatment and a second biopsy at Cycle 3, Day 1 (C3D1; ±5 days) after starting treatment. Biopsies are optional in Part 1.

### II. Number of Participants

The total number of participants in this study will be up to approximately 220 participants. The total number of participants treated in Part 1 will be up to 24 participants (up to 12 participants per dose arm). If one arm is closed prematurely, the remaining participants might be enrolled to backfill remaining or potential newly opened cohorts to obtain additional safety, PK, and PD data.

In Part 2, up to approximately 196 participants will be enrolled, as shown below. A separate interim analysis will be performed after enrollment of at least 17 and up to 21 participants per arm.

Post-IO NSCLC monotherapy (doses to be determined [TBD]): 40 participants

Post-IO NSCLC Compound A (doses TBD) plus docetaxel (75 mg/m² every 3 weeks [Q3W]): 52 participants

Post-IO NSCLC Compound A (doses TBD) plus nivolumab (360 mg Q3W): 52 participants

Second line or later (2L+) mTNBC Compound A (doses TBD) plus capecitabine (1250 mg/m2 twice daily [BID] 2 weeks on and 1 week off): 52 participants

### III. Study Population

### Key Inclusion Criteria

Participant must be 18 years of age or older at the time of signing the informed consent form (ICF).

Participants in Part 1 must have histologic or cytologic confirmation of NSCLC, mTNBC, SCCHN, PAAD, RCC, MSS CRC, or sarcoma, that are advanced (metastatic, recurrent, and/or unresectable) with measurable disease per RECIST v1.1. In Part 2, only participants with histologic confirmation of advanced NSCLC or mTNBC with measurable disease per RECIST v1.1 are eligible.

In Part 2, archival biopsy collected within 3 months of Screening with no intervening therapy (formalin-fixed, paraffin embedded [FFPE] blocks or a minimum of 20 freshly cut unstained FFPE slides with an associated pathological report) or fresh biopsy collection at Screening and fresh biopsy collection at C3D1 (±5 days) are mandatory, while it is strongly encouraged but optional at progression. Therefore, the participant in Part 2 must have a suitable tumor lesion for the biopsy procedure, as judged by the investigator, in order to be eligible for the study.

Eastern Cooperative Oncology Group (ECOG) Performance Status of 0 or 1.

Participants in Part 1 must have received, be refractory to, not be a candidate for, or be intolerant of existing therapy(ies) known to provide clinical benefit for the condition of the participant.

Participants with NSCLC in both Parts 1 and 2 must have histologically confirmed advanced NSCLC per the 8th International Association for the Study of Lung Cancer (IASLC) classification of squamous or non-squamous histology that is resistant or refractory to antiprogrammed cell death 1 (PD-1)-based or anti-programmed cell death ligand 1 (PD-L1)-based treatment. Cytologic confirmation is acceptable in Part 1 of the study. Participants with NSCLC in Part 2A must have experienced radiographically documented progressive disease while on treatment or up to 3 months after the last dose of anti-PD-1 or anti-PD-L1 therapy.

Participants with mTNBC in both Part 1 and Part 2B of the study must have progressed, be intolerant, refractory, or not candidates for at least 2 lines of treatment known to provide clinical benefit in the advanced setting. Participants in Part 2B must not have been exposed to capecitabine in the metastatic setting. Triple negative status will be defined as estrogen receptor and progesterone receptor≤1% by immunohistochemistry (IHC) and human epidermal growth factor receptor 2 (HER2) negative, per American Society of Clinical Oncology/College of American Pathologists (ASCO/CAP) guidelines.

### Key Exclusion Criteria

Participants with primary central nervous system (CNS) disease, or tumors with CNS metastases as the only disease site, will be excluded. Participants with controlled brain metastases, however, will be allowed to enroll. Controlled brain metastases are defined as no radiographic progression for at least 4 weeks following radiation and/or surgical treatment (or 4 weeks of observation if no intervention is clinically indicated), no longer taking steroids for at least 2 weeks prior to first dose of study intervention, and with no new or progressive neurological signs and symptoms.

Participants with concurrent malignancy or history of prior malignancy active within 2 years (except history of early-stage basal/squamous cell skin cancer or non-invasive or in situ cancers who have undergone definitive treatment) are excluded unless treatment was completed at least 2 years before randomization and the participant has no evidence of disease.

Participants with an active, known, or suspected autoimmune disease in Part 1 and Part 2A. Participants with type 1 diabetes mellitus, hypothyroidism only requiring hormone replacement, skin disorders (such as vitiligo, psoriasis, or alopecia) not requiring systemic treatment, or conditions not expected to recur in the absence of an external trigger are permitted to enroll.

Participants with a condition requiring systemic treatment with corticosteroids (>10 mg daily prednisone equivalent) within 14 days or other immunosuppressive medications within 30 days of randomization. Inhaled or topical steroids and adrenal replacement steroid doses>10 mg daily prednisone equivalent are permitted in the absence of active autoimmune disease.

Participants with NSCLC with known epidermal growth factor receptor (EGFR) or V-raf murine sarcoma viral oncogene homolog B1 (BRAF) V600E mutations, or anaplastic lymphoma kinase (ALK) or receptor tyrosine kinase (ROS1) translocations sensitive to available targeted inhibitor therapy are excluded. All participants with non-squamous histology must have been tested for the above mutations and translocations (e.g., with a United States [US] Food and Drug Administration [FDA]-approved test). Participants with nonsquamous histology and unknown mutational status are excluded.

Participants with CRC must have been tested for MSS status, defined as expression of MutL protein homolog 1 (MLH1), MutS homolog 2 (MSH2), MutS homolog 6 (MSH6), PMS1 homolog 2 (PMS2), or polymerase chain reaction (PCR)-based microsatellite instability (MSI) testing by IHC or absence of instability in microsatellite markers by PCR as determined by a local laboratory. Participants with MSI-high or deoxyribonucleic acid (DNA) mismatch repair (MMR) deficiency are excluded. Kirsten rat sarcoma viral oncogene homolog (KRAS) and BRAF status, if known, should be documented. If unknown, participants must consent to test their submitted archived tumor tissue sample (block or unstained slides, collected within 3 months of Screening with no intervening therapy).

### Intervention Groups and Duration:

Part 1 will evaluate increasing doses of Compound A monotherapy (400 mg once daily [QD] and 600 mg QD), administered orally (PO) in successive cohorts.

Participants in Part 2 will receive Compound A at selected dose(s) found to be safe in Part 1.

In Part 2A, participants will receive Compound A QD monotherapy or in combination with docetaxel 75 mg/m² Q3W, administered intravenously (IV) over 1 hour followed by a 60-minute observation period; or nivolumab 360 mg Q3W, administered IV over 30 minutes followed by a 60-minute observation period.

In Part 2B, participants will receive Compound A QD in combination with capecitabine tablets (1250 mg/m²) that will be swallowed whole with water within 30 minutes after a meal BID (morning and evening) for 2 weeks followed by a 1-week rest period.

TABLE 1
Study Intervention
Study Intervention

| Intervention Name | Unit Dose Strength |
|---|---|
| Compound A | 200 mg, 400 mg |
| Docetaxel | 20 mg/2 mL, 80 mg/8 mL, 160 mg/16 mL |
| Nivolumab | 40 mg/4 mL, 100 mg/10 mL, 240 mg/24 mL |
| Capecitabine | 150 mg, 500 mg |

### Statistical Methods:

It is anticipated that Compound A, administered alone and in combination with chemotherapy or nivolumab, will demonstrate adequate safety and tolerability, as well as a favorable benefit/risk profile, to support further clinical testing. No prospective hypotheses are being formally evaluated.

The primary endpoint (incidence of adverse events [AEs], serious AEs [SAEs], dose-limiting toxicities [DLTs], AEs leading to discontinuation, and death) will be analyzed up to 30 days for all treatments except in combination with nivolumab, which will be analyzed for up to 100 days after the last dose of study treatment. DLT rate will be analyzed by dose level and frequency distribution of treated participants with AE using the highest National Cancer Institute (NCI) Common Terminology Criteria for Adverse Events (CTCAE) v5.0 grade.

In Part 2, an interim analysis will be performed for at least 17 and up to 21 participants per arm after sufficient follow-up to investigate the objective response rate (ORR) is obtained. The statistical analysis plan (SAP) will further describe the planned interim analyses.

### BRIEF SUMMARY

The purpose of this study is to assess the safety and tolerability of Compound A as monotherapy and in combination with chemotherapy or nivolumab in participants with advanced solid tumors. Study details include:

Study Intervention Duration: Treatment will be administered for up to 2 years per participant.

Study Duration: The study will last approximately 6 years from first participant first visit (FPFV) in Part 1 through the end of Follow-up for all participants in Part 2.

Study Visit Frequency: Participants will have weekly visits through Week 7. Follow-up visits will be at Days 30 and 100 after the last dose of study treatment.

### Example 2: Pharmacologic Effects of Compound 1 on Tumor Cell Sensitivity to Chemotherapy Agents

### I. Objective:

The objective of this study was to evaluate the effect of Compound 1 treatment on the potency of paclitaxel and 5-Fluorouracil (5-FU) chemotherapy agents across a panel of tumor cell lines.

### II. Study Design

Five tumor cell lines (4T1.2, A549, EMT6, NCI-H460, and HT29) were treated with a range of paclitaxel or 5-FU concentrations in the presence of dimethlylsulfoxide (DMSO), 5 μM Compound 1, or 20 μM Compound 1. Cells were imaged 4 days after treatment and percent confluence was assessed by image quantification. EC50 values for cell confluence were used to assess chemotherapy potency. Eleven patient-derived tumor organoids (MAXFTN449, MAXFTN2990, PAXF1657, PAXF2131, CXF1256, CXF2129, CXF2061, LXFE2478, LXFE690, LXFA677, LXFA644) were seeded in a 3D matrix and treated with a range of paclitaxel, 5-FU, SN38, Gemcitabine, oxaliplatin, and carboplatin concentrations in the presence of DMSO, 5 μM Compound 1, or 20 μM Compound 1. After 8 to 13 days in culture, colonies were stained, imaged, and counted. A dose response curve was generated for chemotherapy alone or chemotherapy in combination with 5 or 20 μM Compound 1. Combination activity was defined as a percentage of the area under the chemotherapy dose response curve (cAUC) for Compound 1 combination groups compared to the cAUC of chemotherapy alone.

TABLE 2
Tumor Cell Lines

| Cell Line | Type |
|---|---|
| 4T1.2 | Braest (murine) |
| EMT6 | Breast (murine) |
| NCI-H460 | Lung (human) |
| HT-29 | Colorectal (human) |
| A549 Nuclight Red | Lung (human) |

TABLE 3
Organoid Cell Lines

| Cell Line | Type | Site |
|---|---|---|
| LXFE2478 | NSCLC (squamous | Metastasis |
| | subtype) | (Brain) |
| LXFE690 | NSCLC (squamous | Primary |
| | subtype) | |
| LXFA677 | NSCLC | Primary |
| | (adenocarcinoma | |
| | subtype) | |
| LXFA644 | NSCLC | Primary |
| | (adenocarcinoma | |
| | subtype) | |
| MAXFTN2990 | breast cancer (triple | Metastasis |
| | negative) | (Lung) |
| MAXFTN449 | breast cancer (triple | Primary |
| | negative) | |
| CXF1256 | colon cancer | Recurrent |
| CXF2061 | colon cancer | Metastasis |
| | | (Liver) |
| CXF2129 | colon cancer | Metastasis |
| | | (Liver) |
| PAXF1657 | pancreatic cancer | Metastasis |
| | | (Lung) |
| PAXF2131 | Pancreatic cancer | Primary |

Abbreviations: NSCLC, non-small cell lung cance

Organoid Viability Assessment

Compound Handling

For combination experiments, working stock solutions of all compounds were prepared in DMSO at concentrations of 660-fold the highest test concentration used and stored at −20° C. Platin compounds were prepared freshly on each treatment day. On each day of an experiment, an aliquot of the working stock solutions was used and stored at room temperature prior to and during treatment. First, 1:2 dilutions of 660-fold concentrated working stocks were prepared in DMSO (in case of monotherapy controls), or equal amounts of the compound working stocks were mixed (in case of drug combinations). Then, different dilutions of these 330-fold concentrated solutions were prepared in DMSO to give an 8×2 matrix combination layout with conditions. The DMSO dilutions were further diluted 1:22 into cell culture medium (IMDM, supplemented with 20% (v/v) fetal bovine serum, and 50 μg/mL gentamicin) in an intermediate dilution plate. Finally, 10 μL taken from the intermediate dilution plate were transferred to 140 μL of cells and medium per well of the final assay plate. Thus, the DMSO dilutions used for the mono- and the combination-treatment were diluted 1:660, and the DMSO concentration in the assay was 0.3% v/v.

Preparation of Single Cell Suspensions from Human Tumor Xenografts

Tumors were passaged as subcutaneous xenografts in NMRI nu/nu mice. At a tumor volume of 400 to 1000 mm³, tumor-bearing mice were euthanized, and tumors were collected under sterile conditions without delay. Tumors were mechanically disaggregated and subsequently incubated with an enzyme cocktail consisting of collagenase type IV (41 U/mL), DNase I (125 U/mL), hyaluronidase type III (100 U/mL), and dispase II (1 U/mL) in RPMI 1640 medium at 37° C. for 60 to 120 minutes. Cells were passed through sieves of 100 μm and 40 μm mesh size, and washed with RPMI 1640 medium. The percentage of viable cells was determined in a Neubauer-hemocytometer using trypan blue exclusion. Aliquots of the cells were frozen down and stored in liquid nitrogen vapor phase. On each day of an experiment, a frozen aliquot of tumor cells was thawed and used for preparation of assay plates.

3D Clonogenic Assay

The clonogenic assay was carried out in a 96 well plate format using ultra low attachment plates. For each test, plates were prepared as follows: each test well contained a layer of semi-solid medium with tumor cells (50 μL), and a second layer of medium supernatant with or without test compound (100 μL). The cell layer consisted of 7.5×103 to 12.5×103 tumor cells per well, which were seeded in 50 μL/well cell culture medium (IMDM, supplemented with 20% (v/v) fetal bovine serum, 50 μg/mL gentamicin, and 0.4% (w/v) agar). After 24 hours, the soft-agar layer was covered with 90 μL of the same culture medium without agar, and 10 μL of control medium or test compound after serial dilution in DMSO for the duration of the experiment (continuous exposure, 100 μL drug overlay). Every plate included six untreated control wells and drug-treated groups in a layout. Cultures were incubated at 37° C. and 7.5% CO₂ in a humidified atmosphere for 8 to 13 days and monitored closely for colony growth using an inverted microscope. Within this period, ex vivo tumor growth led to the formation of colonies with a diameter of >50 μm (area>2000 μm²). At the time of maximum colony formation, vital colonies were stained for 48 hours with a sterile aqueous solution of 2-(4-iodophenyl)-3-(4-nitrophenyl)-5-phenyltetrazolium chloride (INT, 1 mg/mL, 25 μL/well), and colony counts were performed with an automatic image analysis system.

Data Analysis

For 2D viability analyses, viability data were normalized to relative to untreated samples (100%) and media with no cells (0%). EC50 curves were determined using the non-linear curve fit in GraphPad Prism 8 on log transformed values. For 3D colony formation assays, colony numbers were normalized to untreated samples (100%) and samples treated with 100 μM sunitinib (0%). Area under the chemotherapy dose response curve (cAUC) was determined using the area under the curve function in GraphPad Prism 8. cAUC reduction for Compound 1 treatment was defined as the percent reduction relative to chemotherapy alone.

### III. Results

A. Paclitaxel Combinations with Compound 1 in 2D Cell Line Viability Assay

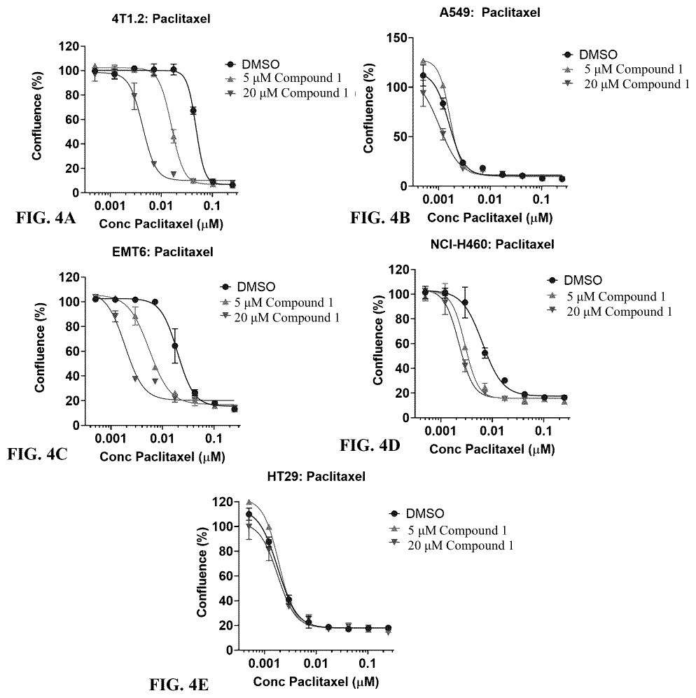

The effects of Compound 1 on the potency of paclitaxel in 4T1.2, A549, EMT6, NCI-H460 and HT29 as measured by cell confluence 4 days after drug treatment are summarized in FIGS. 4A-4E. A 5 and 20 μM dose of Compound 1 were selected to approximate the plasma concentration of Compound 1 at Cmax (14 μM) and Cavg (4 μM) at the 400 mg dose in human. In 4T1.2, in the absence of Compound 1, paclitaxel had an EC50 value of 0.048 μM. In the presence of 5 or 20 μM Compound 1, the EC50 value for paclitaxel in 4T1.2 was lowered to 0.016 and 0.0034 μM, respectively. In A549, in the absence of Compound 1, paclitaxel had an EC50 value of 0.0016 μM. In the presence of 5 or 20 μM Compound 1, the EC50 value for paclitaxel in A549 remained similar at 0.0016 and 0.0010 μM, respectively. In EMT6, in the absence of Compound 1, paclitaxel had an EC50 value of 0.019 μM. In the presence of 5 or 20 μM Compound 1, the EC50 value for paclitaxel in EMT6 was lowered to 0.0055 and 0.0019 μM, respectively. In NCI-H460, in the absence of Compound 1, paclitaxel had an EC50 value of 0.0065 μM. In the presence of 5 or 20 μM Compound 1, the EC50 value for paclitaxel in NCI-H460 was lowered to 0.0029 and 0.0023 μM, respectively. In HT29, in the absence of Compound 1, paclitaxel had an EC50 value of 0.0018 μM. In the presence of 5 or 20 μM Compound 1, the EC50 value for paclitaxel in HT29 remained similar at 0.0019 and 0.0018 μM, respectively. In 3 of the 5 cell lines tested, 5 μM Compound 1 increased cell line sensitivity to paclitaxel from 2.2 to 3.4-fold relative to cells receiving paclitaxel in the absence of Compound 1. In 3 of the 5 cell lines tested, 20 μM Compound 1 increased cell line sensitivity to paclitaxel from 2.9 to 11-fold relative to cells receiving paclitaxel in the absence of Compound 1.

B. 5-FU Combinations with Compound 1 in 2D Cell Line Viability Assay

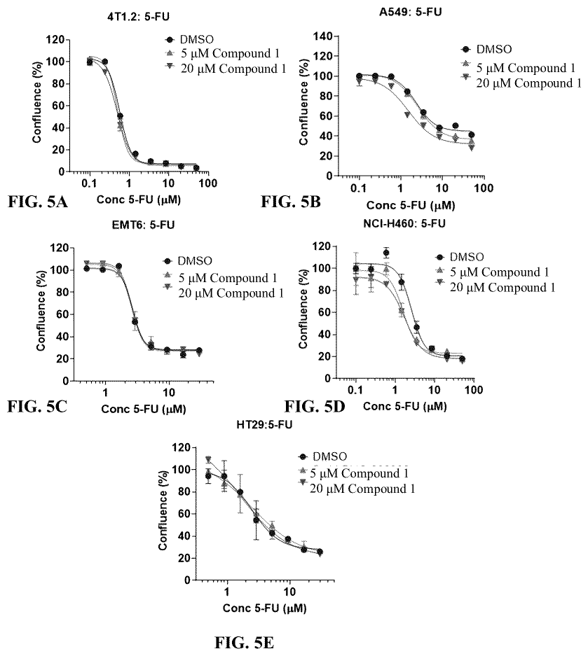

The effects of Compound 1 on the potency of 5-FU in 4T1.2, A549, EMT6, NCI-H460 and HT29 as measured by cell confluence 4 days after drug treatment is summarized in FIGS. 5A-5E. In 4T1.2, in the absence of Compound 1, 5-FU had an EC50 value of 0.57 μM. In the presence of 5 or 20 μM Compound 1, the EC50 value for 5-FU in 4T1.2 remained similar at 0.50 and 0.49 μM, respectively. In A549, in the absence of Compound 1, 5-FU had an EC50 value of 2.4 μM. In the presence of 5 or 20 μM Compound 1, the EC50 value for 5-FU in A549 remained similar at 2.6 and 1.5 μM, respectively. In EMT6, in the absence of Compound 1, 5-FU had an EC50 value of 2.7 μM. In the presence of 5 or 20 μM Compound 1, the EC50 value for 5-FU in EMT6 remained similar at 2.5 and 2.6 μM, respectively. In NCI-H460 in the absence of Compound 1, 5-FU had an EC50 value of 2.6 μM. In the presence of 5 or 20 μM Compound 1, the EC50 value for 5-FU in NCI-H460 remained similar at 1.6 and 1.7 μM, respectively. In HT29, in the absence of Compound 1, 5-FU had an EC50 value of 2.5 μM. In the presence of 5 or 20 μM Compound 1, the EC50 value for 5-FU in HT29 remained similar at 2.5 and 1.6 μM, respectively. In all 5 cell lines, the potency of 5-FU in the presence of Compound 1 remained within 2-fold of the 5-FU potency in the absence Compound 1 co-treatment.

### C. 3D Clonogenic Assay Results

1. Monotherapy Activity of Compound 1 in Clonogenic Assay with Patient Derived Organoids

The mean and standard deviation of percent colony reduction in patient organoid cultures treated with 5 or 20 μM Compound 1 relative to DMSO treated controls 8 to 13 days after cell seeding are summarized in Table 4. The statistical significance for each measurement relative to vehicle control was calculated by a student t-test, and the P-values are also listed in Table 4. After treatment with 5 μM Compound 1, 8 out of 11 organoid cultures had an average percent colony reduction greater than 20%, with a median 39% reduction relative to DMSO. After treatment with 20 μM Compound 1, 10 out of 11 organoid cultures had an average percent colony reduction greater than 20%, with a median 34% reduction relative to DMSO.

TABLE 4
Effect of Compound 1 on Colony Formation
in Patient-derived Tumor Organoids
% Reduction in Colony Formation with
Compound 1 compared with DMSO

| | | | | | Compound 1, | |
|---|---|---|---|---|---|---|
| | Compound 1, 5 μM | | | | 20 μM | |
| | | Std | P value | | Std | |
| Organoid | Mean | Dev | (vs DMSO) | Mean | Dev | P value |
| MAXFTN449 | 56% | 7% | 2.9 × 10¹² | 54% | 4% | 2.1 × 10⁻¹² |
| MAXFTN2990 | −8% | 7% | 1.2 × 10⁻⁰² | 34% | 4% | 1.8 × 10⁻¹¹ |
| PAXF1657 | 31% | 6% | 1.1 × 10⁻⁰⁵ | 27% | 7% | 7.0 × 10⁻⁰⁵ |
| PAXF2131 | 62% | 4% | 1.1 × 10⁻⁰⁸ | 68% | 4% | 3.2 × 10⁻⁰⁹ |
| CXF1256 | 90% | 1% | 2.7 × 10⁻¹⁶ | 92% | 3% | 2.0 × 10⁻¹⁶ |
| CXF2129 | 56% | 12% | 3.7 × 10⁻⁰⁸ | 49% | 6% | 1.1 × 10⁻⁰⁷ |
| CXF2061 | 8% | 7% | 5.9 × 10⁻⁰² | 9% | 7% | 3.3 × 10⁻⁰² |
| LXFE2478 | 39% | 9% | 6.2 × 10⁻⁰⁹ | 28% | 8% | 1.2 × 10⁻⁰⁶ |
| LXFE690 | 73% | 3% | 5.1 × 10⁻¹⁵ | 79% | 3% | 8.7 × 10⁻¹⁶ |
| LXFA677 | 27% | 3% | 1.9 × 10⁻⁰⁶ | 32% | 8% | 1.3 × 10⁻⁰⁶ |
| LXFA644 | 12% | 7% | 9.5 × 10⁻⁰³ | 24% | 6% | 3.1 × 10⁻⁰⁵ |

Abbreviations: DMSO, dimethlylsulfoxide; Std Dev, standard deviation.

2. Chemotherapy Combinations with Compound 1 in Clonogenic Assay with Breast, Lung, Pancreatic, and Colorectal Patient Derived Organoids

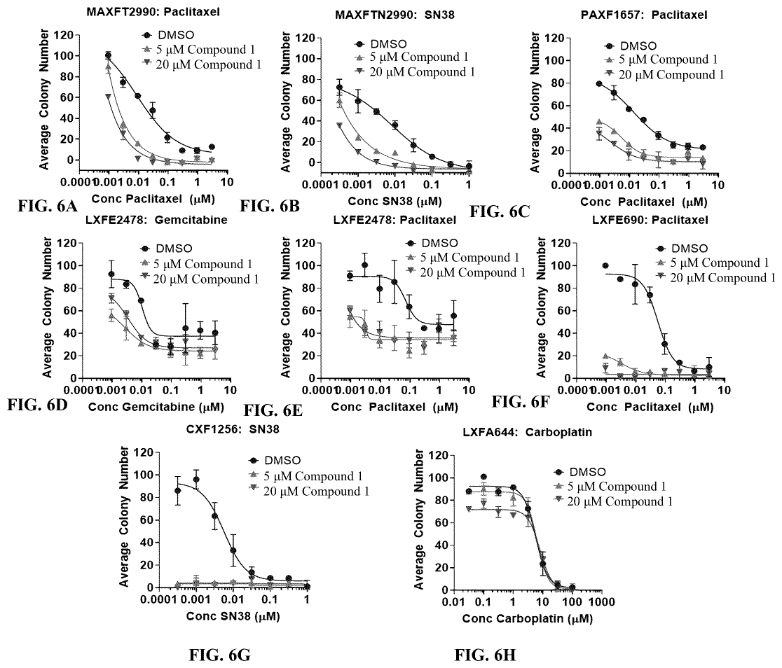

The combination activity observed on tumor colony formation when 5 or 20 μM Compound 1 is added to a dose titration of Paclitaxel, 5-FU, SN38, oxaliplatin, carboplatin, or gemcitabine in a variety of patient derived organoids grown in 3D culture is summarized in Table 5. After 8 to 13 days in culture, colonies were stained, imaged, and counted. A dose response curve was generated for chemotherapy alone or chemotherapy in combination with 5 or 20 μM Compound 1 (FIGS. 6A-6H). Combination activity was defined as the percent reduction of the cAUC for Compound 1 combination groups compared to the cAUC of chemotherapy alone with a value of 0 indicating no combination activity and a value of 100 indicating strong combination activity. Among the 29 combination treatments tested, 23 resulted in greater than 20% reduction in cAUC with either the 5 or 20 μM Compound 1 combination groups compared to the cAUC for chemotherapy alone (Table 5). Among the chemotherapy combinations, paclitaxel frequently exhibited combination activity in organoids, with 42%, 77%, 64%, 71%, 45%, and 94% cAUC reduction relative to chemotherapy alone in MAXFTN449, MAXFTN2990, PAXF1657, PAXF2131, LXFE2478, and LXFE690, respectively, with 20 μM Compound 1 (Table 5, FIG. 6A, FIG. 6C, FIG. 6E, FIG. 6F). Notably, the combination activity observed with paclitaxel in MAXFTN2990, PAXF1657, and LXFE690, was markedly higher than would be predicted by single agent Compound 1 alone (Table 4, Table 5, FIG. 6A, FIG. 6C, FIG. 6F). SN38 induced combination activity of 46%, 74%, 91%, 33%, and 26% cAUC reduction relative to chemotherapy alone in MAXFTN449, MAXFTN2990, CXF1256, CXF2129, and CXF2061, respectively, with 20 μM Compound 1 (Table 5, FIG. 6B, FIG. 6G). In MAXFTN2990, the combination activity with SN38 was markedly higher than would be predicted by single agent Compound 1 alone (Table 4, Table 5, FIG. 6B). In LXFE2478, gemcitabine exhibited combination activity with a 41% cAUC reduction at the 5 μM Compound 1 dose (Table 5, FIG. 6D). For organoids highly sensitive to Compound 1 monotherapy, such as CXF1256, minimal colonies were detected even at low chemotherapy concentrations due to strong anti-tumor activity even at the lower 5 μM Compound 1 dose (Table 5, FIG. 6G). In these settings, combination activity must be assessed at lower Compound 1 concentrations. For some combinations, for example, carboplatin with 5M Compound 1 in LXFA644, cAUC reduction was 50 (Table 5, FIG. 611). Overall, Compound 1 demonstrated broad activity across multiple chemotherapies and cancer types.

TABLE 5
Chemotherapy Combinations with Compound 1 in a
Clonogenic Assay in Patient Derived Organoids

| | | Combination | Combination |
|---|---|---|---|
| | | activity 5 μM | activity 20 μM |
| | | Compound 1 | Compound 1 |
| | Combination | (cAUC % | (cAUC % |
| Organoid | Chemotherapy | reduction) | reduction) |
| MAXFTN449 | Paclitaxel | 57% | 42% |
| | SN38 | 58% | 46% |
| | 5-FU | 29% | 14% |
| MAXFTN2990 | Paclitaxel | 63% | 77% |
| | SN38 | 57% | 74% |
| | 5-FU | −25% | 8% |
| PAXF1657 | Paclitaxel | 50% | 64% |
| | 5-FU | 32% | 32% |
| PAXF2131 | Paclitaxel | 58% | 71% |
| | 5-FU | 52% | 67% |
| CXF1256 | SN38 | 90% | 91% |
| | 5-FU | 78% | 79% |
| | Oxaliplatin | 70% | 63% |
| CXF2129 | SN38 | 44% | 33% |
| | 5-FU | 42% | 31% |
| | Oxaliplatin | 37% | 9% |
| CXF2061 | SN38 | 22% | 26% |
| | 5-FU | 25% | 18% |
| | Oxaliplatin | 13% | −18% |
| LXFE2478 | Gemcitabine | 41% | 30% |
| | Paclitaxel | 46% | 45% |
| | Carboplatin | 27% | 3% |
| LXFE690 | Gemcitabine | 59% | 61% |
| | Paclitaxel | 87% | 94% |
| | Carboplatin | 73% | 80% |
| LXFA677 | Gemcitabine | 7% | 15% |
| | Carboplatin | 7% | 14% |
| LXFA644 | Gemcitabine | 3% | 20% |
| | Carboplatin | 5% | 19% |

Dose range tested for chemotherapy were as follows: Paclitaxel (0.00095-3 μM); 5-FU (00.0316-100 μM); SN38 (0.000316-1 μM); Oxaliplatin (0.0316-100 μM); Gemcitabine (0.00095-3 μM); Gemcitabine (0.00095-3 μM). Abbreviations: 5-FU, 5-Fluorouracil; cAUC, area under the chemotherapy dose response curve.

### IV. Conclusion

Compound 1 consistently sensitized tumor cell lines and patient derived tumor organoids to chemotherapy in 2D and 3D culture. Combination activity was observed for all chemotherapy agents tested, particularly in patient derived organoids. Paclitaxel appears to be most frequently and most significantly impacted by Compound 1 treatment. These data suggest Compound 1 can directly impact tumor cells to enhance the activity of chemotherapy in patients with breast, lung, and colorectal tumors.

### Example 3: In Vitro Pharmacologic Characterization of Compound 1 in Fibroblasts, Tumor Cells, and Macrophages

### I. Objective

The objective of this study was to evaluate the functional effects of Compound 1 treatment on fibroblasts, tumor cells, and macrophages.

### II. Study Design

Primary human fibroblasts from normal lung or non-small cell lung cancer were treated with varying concentrations of Compound 1 and stimulated with transforming growth factor beta (TGF-β). Transcriptional markers of fibroblast activation were assessed by quantitative polymerase chain reaction (qPCR). Human tumor cell lines were treated with varying concentrations of Compound 1 and stimulated with TGF-β. Transcriptional markers of epithelial-to-mesenchymal transition were assessed by qPCR. Primary human monocytes were differentiated into macrophages and treated with varying concentrations of Compound 1. C—C motif chemokine ligand 2 (CCL2) secretion was measured by enzyme-linked immunosorbent assay (ELISA).

Methods

Fibroblast Activation Assay

Capillary Western

Normal human lung fibroblasts (NHLF) and lung cancer-associated fibroblasts (CAF) were maintained in their respective vendor recommended media. For the purposes of comparing the two cell lines, NHLF and lung CAF were both adapted to DMEM+10% fetal bovine serum (FBS) and to RPMI+10% FBS prior to use in the activation assay. Cells were seeded (150,000 NHLF and lung CAF cells) in 12 well plates and allowed to adhere overnight at 37° C., 5% CO₂. The next day, cells were pre-treated for 30 minutes at 37° C. with DMSO or Compound 1 at 30 μM, 7.5 μM, 1.875 μM, 0.469 μM, 0.117 μM, 0.029 μM, and 0.007 μM. Cells were stimulated with 10 ng/mL recombinant TGF-β and protein lysates were collected after two hours. After a phosphate-buffered saline (PBS) rinse step, fresh lysis buffer consisting of M-PER™ and phosphatase inhibitor PhosSTOP™ and cOmplete™ Mini EDTA-Free Protease Inhibitor Cocktail was added to the treated cells. Lysates were transferred into 1.5 mL microcentrifuge tubes and centrifuged at 10,000×g for 10 minutes at 4° C. Protein quantitation of cell lysates was performed using the Pierce™ BCA Protein Assay kit and absorbance was read using a SpectraMax® microplate reader. Samples were diluted five-fold to maintain within the range of accuracy for the assay. Cell lysates were normalized to 0.4 μg/L using M-PER™ lysis buffer. WES/JESS Capillary Westerns were performed per vendor protocol, using WES/JESS Separation Module 12-230 kDa 8×25 cartridges and anti-Rabbit Detection Modules on a WES or JESS Simple Western Instrument. Instrument parameters were as follows: vendor's size assay protocol for 12-230 kDa range, 25 sample cartridge, separation time of 25 minutes, separation voltage of 375 V, Antibody Diluent time of 5 minutes, primary diluent time of 30 minutes, secondary antibody time of 30 minutes, and detection profile of high dynamic range (HDR) chemiluminescence. The following primary antibodies were used: 1:50 phospho-c-Jun (Ser73) (D47G9) XP Rabbit mAb, 1:50 c-Jun (60A8) Rabbit mAb, and 1:200 GAPDH (14C10) Rabbit mAb. Compass for Simple Western Software (ProteinSimple, Version 6.0.0, San Jose, CA) was used for data analysis. Peak Area data was collected and normalized to total c-Jun, and the ratio was plotted.

NHLF and Lung CAF Samples for Ribonucleic Acid

Cells were seeded (75,000 NHLF and lung CAF cells) into 12 well plates with RPMI+10% FBS and allowed to adhere overnight at 37° C., 5% CO2. After one day of serum starvation, cells were pre-treated for 1 hour with DMSO or Compound 1 at 30 μM, 10 μM, 3 μM, 1 μM, or 0.3 μM and then stimulated with 10 ng/mL TGF-β. The 24-hour Compound 1 treated samples were harvested for ribonucleic acid (RNA) extraction.

RNA Extraction

Cells were directly lysed in Qiagen™ Buffer RLT Plus with 1% 2-Mercaptoethanol added. Total RNA from tumor samples and cell lines was isolated using Qiagen™ AllPrep® DNA/RNA 96 Kit according to manufacturer's protocol. Isolated RNA was analyzed with NanoDrop™ ONE Spectrophotometer for concentration. The RNA integrity was also analyzed on Agilent TapeStation 4200.

Reverse Transcription

Complimentary deoxyribonucleic acid (cDNA) was prepared by using Invitrogen™ SuperScript™ III First-Strand Synthesis SuperMix for qRT-PCR. Up to 1 μg of RNA was prepared according to manufacturer's protocol, using Applied Biosystems™ Veriti™ 96-well thermal cycler. The reverse transcription (RT) program used for cDNA synthesis:
1. 25° C., 10 minutes
2. 50° C., 30 minutes
3. 85° C., 5 minutes
4. Add E. Coli RNase H, 37° C., 20 minutes
5. Hold at 4° C. Quantitative Reverse Transcription Polymerase Chain Reaction (qRT-PCR).

The cDNA was diluted 1:3 fold in nuclease-free water for quantitative polymerase chain reaction (qPCR) setup. Human and mouse TaqMan™ assays for targeted genes were ordered through Applied Biosystems™. TaqMan™ assay selection was based on manufacturer's recommendation. The qRT-PCR was set up using Applied Biosystems™ TaqMan™ Fast Advanced Master Mix according to manufacturer's protocol. The qRT-PCR was run on QuantStudio™ 12 Flex with fast run mode:
1. 95° C., 20 seconds
2. 95° C., 1 second
3. 60° C., 20 seconds
4. Repeat steps 2-3, 39 times

The qPCR data was analyzed, and all cycle threshold (Ct) values were normalized to glyceraldehyde-3-phosphate dehydrogenase (GAPDH) (ΔCt). Analysis was plotted and presented using GraphPad Prism (GraphPad, Version 8.4.1, San Diego, CA).

Evaluation of Mesenchymal Transcripts in Tumor Cells Stimulated with TGF-β

Cells were seeded (100,000 A549 Nuclear Red cells from Sartorius) into 12 well plates with F-12K media+10% FBS and allowed to adhere overnight. Following a day of serum starvation, cells in F-12K media were pre-treated for 1 hour with DMSO or Compound 1 at 30 μM, 15 μM or 5 μM and then stimulated with 10 ng/mL TGF-β. The 24-hour Compound 1 treated samples were harvested for RNA extraction.

Chemokine Secretion Assay in Macrophages

Generation of Primary Human Macrophages from Blood

Peripheral blood mononuclear cells (PBMCs) were isolated from human blood using aseptic conditions. CD14+ monocytes were enriched from PBMC using Miltenyi Biotech kit (using CD14 microbeads). Fresh CD14+ monocytes were differentiated to macrophages using complete RPMI1640 medium with M-CSF recombinant human protein at the final concentration of 50 ng/mL. At Day 3-4, media containing M-CSF was added on monocytes/macrophages and incubated for 6-7 days in 5% CO₂ incubator at 37° C. 10 mL 10 mM EDTA or Detachin was added to the culture dish containing macrophages, and let sit for 20-30 minutes, or until cells dissociate from the dish at room temperature. Macrophages were collected and washed in a 50 mL conical tube and centrifuged at 300-400×g for 4-5 minutes at room temperature. The supernatant was discarded and primary macrophages (30-50 k)/100 μL/well were plated on a U-bottom 96 well plate. These cells were treated with DMSO or Compound 1.

ELISA of Human CCL2 from the Macrophage Culture Supernatant

Human macrophages were treated with various concentrations of either DMSO or Compound 1 for 48 hours. The supernatant was collected and assayed using Human CCL2/MCP-1 Quantikine ELISA kit according to the manufacturer's instructions. Cell titer glow (CTG) was used to detect the effect of Compound 1 on the viability of primary human macrophages. 100 μL of CTG (Promega, #G8461) was added and the assay was performed according to manufacturer's instructions.

Data Analysis

Compass for Simple Western Software (ProteinSimple, Version 6.0.0, San Jose, CA) was used for data analysis of capillary western data. Phospho-c-Jun (Ser73) peak area values were calculated using Gaussian distribution and were normalized to peak area values of total c-Jun. Normalized phospho-c-Jun peak area was plotted and EC50 values were obtained using the nonlinear regression equation for “log(inhibitor) vs normalized response” in GraphPad Prism (GraphPad, Version 8.4.1, San Diego, CA). Statistical analysis was run using two-tailed student's t-test.

### III. Results

A. Pharmacologic Effect of Compound 1 on Transcriptional Markers of Fibroblast Activation in Normal and Cancer-Associated Fibroblasts Treated with TGF-β

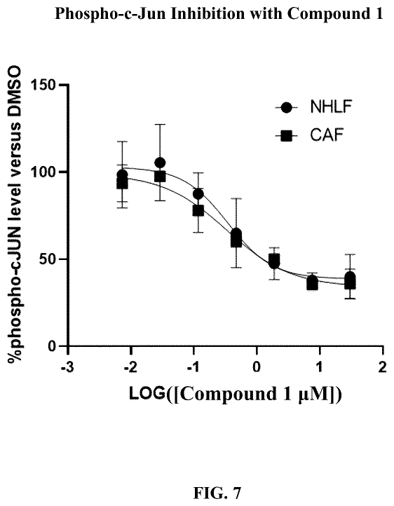

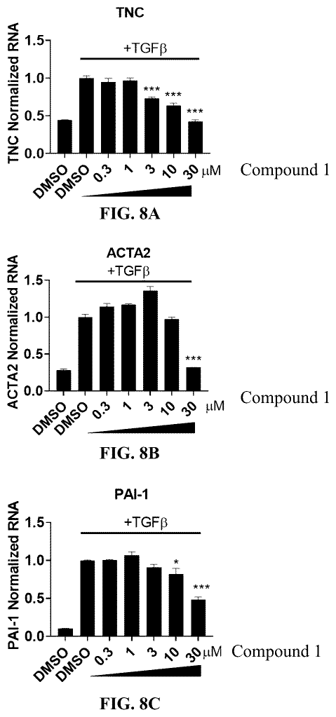

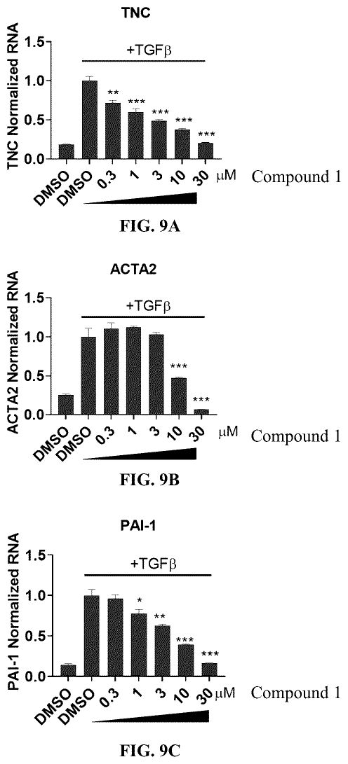

The levels of phospho-c-Jun in NHLF and non-small cell lung CAF in response to TGF-β in the presence of DMSO or varying concentrations of Compound 1 were summarized in FIG. 7. Cells were treated with DMSO or Compound 1 for 1 hour prior to treatment with 10 ng/mL TGF-β for an additional 2 hours. Phospho-c-Jun levels were determined by western blot analysis and were normalized to DMSO treated samples. A dose-dependent reduction in phospho-c-Jun was observed with Compound 1 treatment. The EC50 for Compound 1 on phospho-c-Jun levels was 0.35 and 0.33 μM in NHLF and CAF, respectively. The relative expression of fibroblast activation markers in NHLF and lung CAF in response to TGF-β in the presence of DMSO or varying concentrations of Compound 1 were summarized in FIGS. 8A-8C and FIGS. 9A-9C. Cells were treated with DMSO or Compound 1 for 2 hours prior to treatment with 10 ng/mL TGF-β. The RNA expression was assessed 24 hours after TGF-β treatment. Markers of fibroblast activation, TNC, ACTA2, and PAI-1, were measured relative to housekeeping gene GAPDH and normalized to the expression observed after TGF-β treatment.

In the presence of DMSO, all three activation markers, TNC, ACTA2, and PAI-1, were significantly increased 24 hours after TGF-β stimulation in both normal and CAF from lung, compared to cells that did not receive TGF-β (baseline). In the presence of Compound 1, the TGF-β-induced increase in gene transcripts were dose-dependently reduced. For NHLF, TNC induction was significantly reduced starting at 3 μM Compound 1 with a 48% reduction from peak activation to baseline. At the highest 30 μM dose, TNC in NHLF was lower than samples that never received TGF-β. For CAF, TNC induction was significantly reduced starting at 0.3 μM Compound 1 with a 34% reduction from peak activation relative to baseline. At the highest 30 μM dose, TNC in CAF was reduced 97% from peak activation relative to baseline. For NHLF, ACTA2 induction was significantly reduced only at the highest 30 μM Compound 1 dose with a 95% reduction from peak activation to baseline. For CAF, ACTA2 induction was significantly reduced starting at 10 μM Compound 1 with a 71% reduction from peak activation relative to baseline. At the highest 30 μM dose, ACTA2 in CAF was lower than samples in the absence of TGF-β. For NHLF, PAI-1 induction was significantly reduced starting at 10 μM Compound 1 with a 20% reduction from peak activation to baseline. At the highest 30 μM dose, PAI-1 in NHLF was reduced 57% from peak activation relative to baseline. For CAF, PAI-1 induction was significantly reduced starting at 1 μM Compound 1 with a 25% reduction from peak activation relative to baseline. At the highest 30 μM dose, PAI-1 in CAF was reduced 98% from peak activation relative to baseline.

B. Pharmacologic Effect of Compound 1 on TGF-3-induced Transcripts in A549 Tumor Cells

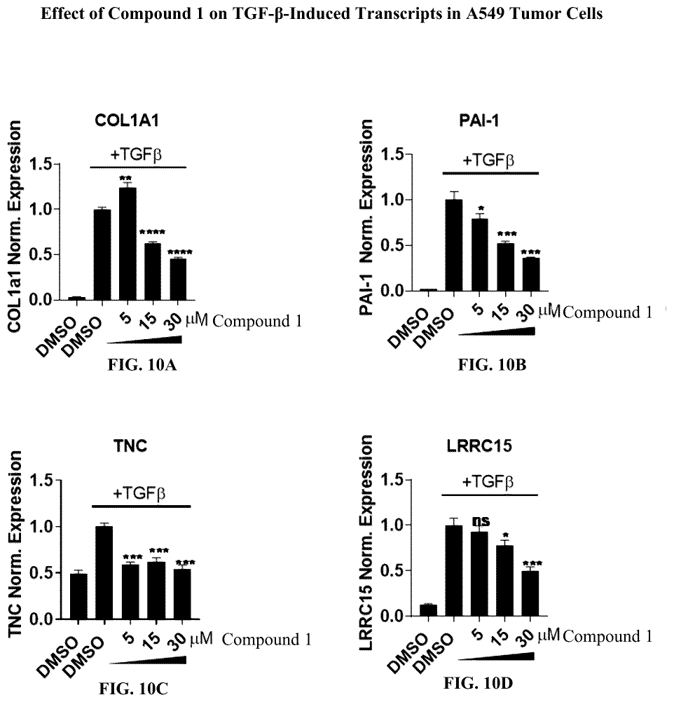

The relative expression of TGFβ-responsive transcripts in A549 lung cancer cells in the presence of DMSO or varying concentrations of Compound 1 were summarized in FIGS. 10A-D. Cells were treated with DMSO or Compound 1 for 2 hours prior to treatment with 10 ng/mL TGF-β. The RNA expression was assessed 24 hours after TGF-β treatment. The TGF-β-responsive transcripts, PAI-1 and COL1A1, TNC, and LRRC15, were measured relative to the housekeeping gene GAPDH and normalized to the expression observed after TGF-β treatment. In the presence of DMSO, all four activation markers, COL1A1, PAI-1, TNC, and LRRC15, were markedly increased 24 hours after TGF-β stimulation in A549 compared to cells that did not receive TGF-β (baseline). Compound 1 dose-dependently reduced the expression of COL1A1 and PAI-1 in A549 cells treated with TGF-β (FIG. 10A and FIG. 10B). Tenascin C was upregulated with TGF-β treatment and inhibited by all doses of Compound 1 tested (FIG. 10C). LRRC15 was upregulated with TGF-β treatment, but dose dependently reduced by Compound 1 (FIG. 10D). At the maximum 30 μM dose of Compound 1, COL1A1, PAI-1, TNC, and LRRC15 were reduced 56%, 64%, 90%, and 57%, respectively, relative to TGF-β stimulation in the presence of DMSO alone.

### C. Pharmacologic Effect of Compound 1 on CCL2 Secretion in Primary Macrophages

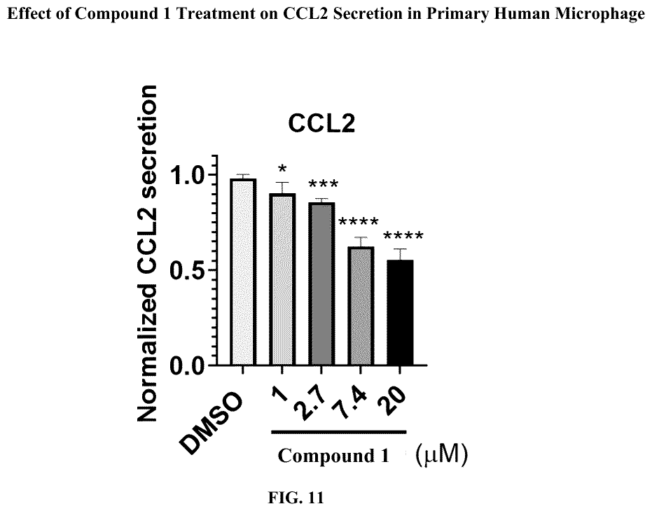

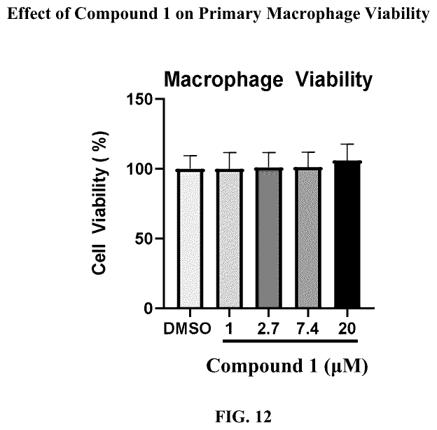

The CCL2 protein levels from the supernatant of primary human macrophages treated with DMSO or varying concentrations of Compound 1 were plotted in FIG. 11. Compound 1 dose-dependently reduced CCL2 secretion by macrophages. At 1 μM Compound 1, there was an 8% reduction in CCL2. At the higher 7.4 and 20 μM doses of Compound 1, CCL2 levels were reduced 36% and 43%, respectively. Macrophage cell viability at the end of the experiment was assessed by Cell Titer Glo and is plotted in FIG. 12. No change in cell viability was observed in the Compound 1 treated cells relative to DMSO at any of the doses tested.

TABLE 6
Summary of Results, In Vitro Nonclinical Pharmacology Studies

| Type of | | Detection | |
|---|---|---|---|
| Study | Test System | method | Noteworthy Findings |
| TGF-β-induced | Primary human | Western Blot: | The EC50 for Compound 1 on phospho-c-Jun |
| c-Jun | fibroblasts from | phospho-c-Jun | levels was 0.35 and 0.33 μM in NHLF and |
| phosphorylation | normal lung or | | CAF, respectively. |
| | NSCLC | | |
| TGF-β-induced | Primary human | RT-PCR: TNC, | Compound 1 dose dependently reduced TGF-β- |
| gene | fibroblasts from | ACTA2, PAI-1 | induced genes PAI-1, TNC, and ACTA2 in |
| transcription | normal lung or | | normal and cancer associated fibroblasts. In |
| | NSCLC | | NHLF stimulated with TGFB, PAI-1, ACTA2, |
| | | | and TNC were reduced 20%, 4.1%, and 66% |
| | | | with 10 μM Compound 1 relative to DMSO, |
| | | | respectively. In CAF stimulated with TGFβ, |
| | | | PAI-1, ACTA2, and TNC were reduced 71%, |
| | | | 71%, and 77% with 10 μM Compound 1 |
| | | | relative to DMSO, respectively. |
| TGF-β-induced | A549 lung | RT-PCR: | Compound 1 dose dependently reduced |
| gene | tumors | COL1A1, | TGF-β-induced genes COL1A1, PAI-1, TNC, |
| transcription | | PAI-1, | and LRRC15 in normal and cancer associated |
| | | TNC, LRRC15 | fibroblasts. At the maximum 30 μM dose of |
| | | | Compound 1, COL1A1, PAI-1, TNC, and |
| | | | LRRC15 were reduced 56%, 64%, 90%, and |
| | | | 57% respectively relative to TGF-β stimulation |
| | | | in the presence of DMSO alone. |
| Cytokine | Primary | ELISA: CCL2 | At 1 μM Compound 1, there was an 8% |
| secretion | human | | reduction in CCL2. At the higher 7.4 and 20 |
| | macrophages | | μM doses of Compound 1, CCL2 levels |
| | | | were reduced 36% and 43%, respectively. |

Abbreviations: ACTA2, smooth muscle actin; CAF, cancer-associated fibroblasts; CCL2, C-C motif chemokine ligand 2; COL1A1, collagen 1 alpha 1; DMSO, dimethyl sulfoxide; ELISA, enzyme-linked immunosorbent assay; LRRC15, leucine-rich repeat containing 15; NHLF, normal human lung fibroblasts; NSCLC, non-small cell lung cancer; PAI-1, plasminogen activator inhibitor 1; RT-PCR, reverse transcription polymerase chain reaction; TGF-β, transforming growth factor beta; TNC, tenascin C.

### IV. Conclusion

Compound 1 pharmacologically inhibited the induction TGF-β responsive transcripts in normal lung fibroblasts, lung CAFs, and A549 tumor cells. The TGF-β activity in the TME is a known driver of tumor fibrosis, a characteristic that is associated with resistance to immune and chemotherapy. In primary human macrophages, Compound 1 also dose-dependently reduces the secretion of CCL2, a chemokine that recruits regulatory T cells to suppress the activity of cytotoxic T cells. Collectively, Compounds 1 reduces mediators of immune suppression on tumor cells, fibroblasts, and macrophages, supporting the use of Compound 1 as a monotherapy or in combination with immune or chemotherapy.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating or managing cancer, comprising administering to a patient having said cancer a therapeutically effective amount of Compound 1 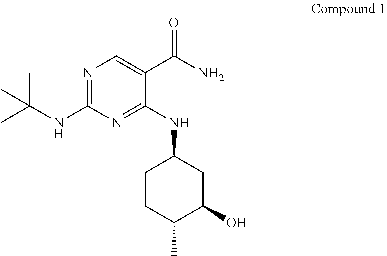 [SMILES: C[C@@H]1CC[C@@H](Nc2nc(NC(C)(C)C)ncc2C(N)=O)C[C@H]1O]
or a pharmaceutically acceptable salt, stereoisomer, tautomer, solid form, polymorph, hydrate, clathrate, or solvate thereof.

2. A method of treating or managing cancer, comprising administering to a patient having said cancer a therapeutically effective amount of (i) Compound 1 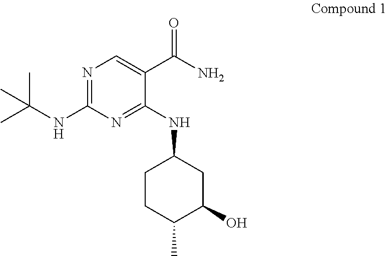 [SMILES: C[C@@H]1CC[C@@H](Nc2nc(NC(C)(C)C)ncc2C(N)=O)C[C@H]1O]
or a pharmaceutically acceptable salt, stereoisomer, tautomer, solid form, polymorph, hydrate, clathrate, or solvate thereof and (ii) nivolumab.

3. A method of treating or managing cancer, comprising administering to a patient having said cancer a therapeutically effective amount of (i) Compound 1 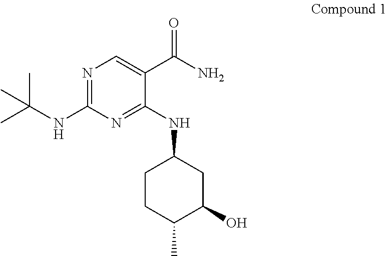 [SMILES: C[C@@H]1CC[C@@H](Nc2nc(NC(C)(C)C)ncc2C(N)=O)C[C@H]1O]
or a pharmaceutically acceptable salt, stereoisomer, tautomer, solid form, polymorph, hydrate, clathrate, or solvate thereof and (ii) chemotherapy.

4. The method of claim 1, wherein the cancer is a solid tumor.

5. The method of claim 4, wherein the solid tumor is lung cancer, breast cancer, squamous cell carcinoma of head and neck, pancreatic adenocarcinoma, pancreatic ductal adenocarcinoma, renal cell carcinoma, colorectal carcinoma, or sarcoma.

6. The method of claim 4, wherein the solid tumor is non-small cell lung cancer, triple negative breast cancer, or microsatellite-stable colorectal carcinoma.

7. The method of claim 6, wherein the triple negative breast cancer is metastatic triple negative breast cancer.

8. The method of claim 4, wherein the solid tumor is metastatic, refractory, recurrent, and/or unresectable.

9. The method of claim 4, wherein the solid tumor is immunotherapy-refractory.

10. The method of claim 3, wherein the chemotherapy is docetaxel or capecitabine.

11. A method for treating or managing a solid tumor, comprising:
(i) identifying a patient having a solid tumor sensitive to treatment with Compound 1, or a pharmaceutically acceptable salt, stereoisomer, tautomer, solid form, polymorph, hydrate, clathrate, or solvate thereof; and
(ii) administering to the patient a therapeutically effective amount of Compound 1, or a pharmaceutically acceptable salt, stereoisomer, tautomer, solid form, polymorph, hydrate, clathrate, or solvate thereof.

12. (canceled)

13. (canceled)

14. The method of claim 11, wherein the solid tumor is metastatic, refractory, recurrent, and/or unresectable.

15. The method of claim 11, wherein the solid tumor is lung cancer, breast cancer, squamous cell carcinoma of head and neck, pancreatic adenocarcinoma, pancreatic ductal adenocarcinoma, renal cell carcinoma, colorectal carcinoma, or sarcoma.

16-19. (canceled)

20. The method of claim 1, wherein Compound 1, or a pharmaceutically acceptable salt, stereoisomer, tautomer, solid form, polymorph, hydrate, clathrate, or solvate thereof, is administered in an amount of from about 200 mg to about 900 mg per day.

21. The method of claim 2, wherein (i) Compound 1, or a pharmaceutically acceptable salt, stereoisomer, tautomer, solid form, polymorph, hydrate, clathrate, or solvate thereof, is administered in an amount of from about 200 mg to about 900 mg per day, and (ii) nivolumab is administered in an amount of about 300 mg to about 400 mg every three weeks.

22. The method of claim 10, wherein (i) Compound 1, or a pharmaceutically acceptable salt, stereoisomer, tautomer, solid form, polymorph, hydrate, clathrate, or solvate thereof, is administered in an amount of from about 200 to about 900 mg per day, (ii) and docetaxel is administered in an amount of from about 50 to about 100 mg/m² every three weeks.

23. The method of claim 10, wherein (i) Compound 1, or a pharmaceutically acceptable salt, stereoisomer, tautomer, solid form, polymorph, hydrate, clathrate, or solvate thereof, is administered in an amount of from about 200 to about 900 mg per day, (ii) and capecitabine is administered in an amount of from about 300 to about 2000 mg/m² twice daily.

24. The method of claim 21, wherein (i) Compound 1, or a pharmaceutically acceptable salt, stereoisomer, tautomer, solid form, polymorph, hydrate, clathrate, or solvate thereof, is administered in an amount of about 200, about 300, about 400, about 500, about 600 mg, about 700 mg, about 800 mg, or about 900 mg per day, and (ii) nivolumab is administered in an amount of about 360 mg every three weeks.

25. The method of claim 21, wherein (i) Compound 1, or a pharmaceutically acceptable salt, stereoisomer, tautomer, solid form, polymorph, hydrate, clathrate, or solvate thereof, is administered orally and (ii) nivolumab is administered intravenously.

26. (canceled)

27. The method of claim 25, wherein (i) Compound 1, or a pharmaceutically acceptable salt, stereoisomer, tautomer, solid form, polymorph, hydrate, clathrate, or solvate thereof, is administered in 200 mg or 400 mg of a tablet and (ii) nivolumab is administered as intravenous infusion of 40 mg/4 ml, 100 mg/10 ml, 240 mg/24 ml, single dose vial.

28. The method of claim 25, wherein (i) Compound 1, or a pharmaceutically acceptable salt, stereoisomer, tautomer, solid form, polymorph, hydrate, clathrate, or solvate thereof, is administered in 600 mg or 800 mg or 900 mg of a tablet and (ii) nivolumab is administered as intravenous infusion of 40 mg/4 ml, 100 mg/10 ml, 240 mg/24 ml, single dose vial.

29. The method of claim 22, wherein (i) Compound 1, or a pharmaceutically acceptable salt, stereoisomer, tautomer, solid form, polymorph, hydrate, clathrate, or solvate thereof, is administered in an amount of about 200, about 300, about 400, about 500, about 600 mg, about 800, or about 900 mg per day, and (ii) docetaxel is administered in an amount of about 60, about 70, or about 75 mg/m² every three weeks.

30. The method of claim 22, wherein (i) Compound 1, or a pharmaceutically acceptable salt, stereoisomer, tautomer, solid form, polymorph, hydrate, clathrate, or solvate thereof, is administered orally, and (ii) docetaxel is administered intravenously.

31. (canceled)

32. The method of claim 30, wherein (i) Compound 1, or a pharmaceutically acceptable salt, stereoisomer, tautomer, solid form, polymorph, hydrate, clathrate, or solvate thereof, is administered in 600 mg, or 800 mg, or 900 mg of a tablet, and (ii) docetaxel is administered as intravenous infusion of 20 mg/2 ml, 80 mg/8 mL, or 160 mg/16 mL multi dose vial.

33. The method of claim 32, wherein (i) Compound 1, or a pharmaceutically acceptable salt, stereoisomer, tautomer, solid form, polymorph, hydrate, clathrate, or solvate thereof, is administered in 200 mg or 400 mg of a tablet, and (ii) docetaxel is administered as intravenous infusion of 20 mg/2 ml, 80 mg/8 mL, or 160 mg/16 mL multi dose vial.

34. The method of claim 23, wherein (i) Compound 1, or a pharmaceutically acceptable salt, stereoisomer, tautomer, solid form, polymorph, hydrate, clathrate, or solvate thereof, is administered in an amount of about 200, about 300, about 400, about 500, about 600 mg, about 800 mg, or about 900 mg per day, and (ii) capecitabine is administered in an amount of about 300, about 400, about 500, about 600, about 700, about 800, about 900, about 1000, about 1100, about 1200 or about 1250 mg/m² twice daily.

35. The method of claim 23, wherein (i) Compound 1, or a pharmaceutically acceptable salt, stereoisomer, tautomer, solid form, polymorph, hydrate, clathrate, or solvate thereof, is administered orally, and (ii) capecitabine is administered orally.

36. (canceled)

37. The method of claim 23, wherein (i) Compound 1, or a pharmaceutically acceptable salt, stereoisomer, tautomer, solid form, polymorph, hydrate, clathrate, or solvate thereof, is administered in 200 mg or 400 mg of a tablet, and (ii) capecitabine is administered in 150 mg or 500 mg of a tablet.

38. The method of claim 4, wherein the solid tumor is metastatic, refractory, recurrent, and/or unresectable.

39. The method of claim 22, wherein the docetaxel is administered in an amount of about 75 mg/m² every three weeks.

40. The method of claim 39, wherein the docetaxel is administered at least 4 to 6 cycles.

41. The method of claim 23, wherein the capecitabine is administered in an amount of about 1250 mg/m² twice daily; or about 1000 mg/m² twice daily.

42. (canceled)

43. The method of claim 41, wherein the capecitabine is administered 2 weeks on and 1 week off.

44. (canceled)

45. The method of claim 21, wherein the solid tumor is immunotherapy refractory non small lung cancer.

46. The method of claim 23, wherein the solid tumor is metastatic triple negative breast cancer.

47. The method of claim 22, wherein the solid tumor is immunotherapy refractory non small lung cancer.

48. The method of claim 2, wherein the solid tumor is lung cancer, breast cancer, squamous cell carcinoma of head and neck, pancreatic adenocarcinoma, pancreatic ductal adenocarcinoma, renal cell carcinoma, colorectal carcinoma, or sarcoma.

49. The method of claim 3, wherein the solid tumor is lung cancer, breast cancer, squamous cell carcinoma of head and neck, pancreatic adenocarcinoma, pancreatic ductal adenocarcinoma, renal cell carcinoma, colorectal carcinoma, or sarcoma.

50. The method of claim 48, wherein the solid tumor is metastatic, refractory, recurrent, and/or unresectable.

51. The method of claim 49, wherein the solid tumor is metastatic, refractory, recurrent, and/or unresectable.
